(19) United States
(12) Patent Application Publication
(10) Pub. No.: US 2024/0293362 A1
(43) Pub. Date: Sep. 5, 2024

(54) Soluble Epoxide Hydrolase Inhibitors and Use Thereof

(71) Applicant: Board of Trustees of Michigan State University, East Lansing, MI (US)

(72) Inventors: Edmund L. ELLSWORTH, Vicksburg, MI (US); Kin Sing Stephen LEE, East Lansing, MI (US)

(73) Assignee: Board of Trustees of Michigan State University, East Lansing, MI (US)

(21) Appl. No.: 18/634,344

(22) Filed: Apr. 12, 2024

Related U.S. Application Data
(63) Continuation of application No. PCT/US22/46421, filed on Oct. 12, 2022.
(60) Provisional application No. 63/255,244, filed on Oct. 13, 2021.

(51) Int. Cl. A61K 31/40; A61K 31/397; A61K 31/403; A61K 31/407; A61K 31/438; A61K 31/439; A61K 31/4439; A61K 31/4468; A61K 31/46; A61P 25/28; C07D 205/04; C07D 207/09; C07D 207/14; C07D 209/52; C07D 211/58; C07D 221/24; C07D 401/12; C07D 451/04; C07D 471/10; C07D 487/04; C07D 487/10

(52) U.S. Cl. CPC A61K 31/40; A61K 31/397; A61K 31/403; A61K 31/407; A61K 31/438; A61K 31/439; A61K 31/4439; A61K 31/4468; A61K 31/46; A61P 25/28; C07D 205/04; C07D 207/09; C07D 207/14; C07D 209/52; C07D 211/58; C07D 221/24; C07D 401/12; C07D 451/04; C07D 471/10; C07D 487/04; C07D 487/10

(57) ABSTRACT

Compounds and methods to treat or prevent CNS diseases, such as Alzheimer's disease and brain injury inflammation, are disclosed herein. The compounds are soluble epoxide hydrolase (sEH) inhibitors and have improved physical properties, blood-brain-barrier (BBB) penetration and long drug-target residence time. Pharmaceutical compositions and kits comprising the compounds are also disclosed herein.

### CROSS-REFERENCE TO RELATED APPLICATION

This patent application claims priority to and the benefit of U.S. Provisional Patent Application No. 63/255,244 filed on 13 Oct. 2021, the entire contents of which are herein incorporated by reference.

### FIELD

The present invention generally relates to soluble epoxide hydrolase (sEH) inhibitors, pharmaceutical compositions thereof, and uses thereof, such as preventing and/or treating central nervous system (CNS) diseases.

### U.S. GOVERNMENT RIGHTS

This invention was made with government support under 1761320 awarded by the National Science Foundation. The government has certain rights in the invention.

### BACKGROUND

The background includes information that may be useful in understanding the present disclosure. It is not an admission that any of the information provided herein is prior art or relevant to the presently claimed invention, or that any publication specifically or implicitly referenced is prior art.

sEH is a cytosolic enzyme that degrades epoxy-polyunsaturated fatty acids (epoxy-PUFAs) to corresponding dihydroxy-PUFAs and its inhibition has shown significant clinical importance for the treatment of inflammation. More recently, inhibition or genetic knockout of sEH alleviates the pathogenesis of multiple CNS disease models including Alzheimer's disease, Parkinson's disease, brain ischemia, etc. Therefore, sEH is a novel therapeutic target for CNS diseases. See Wagner et al., Pharmacol Ther. (2017) 180:62-76. Exemplary sEH inhibitors are also reported in U.S. 2018/0325860 and WO 2019/243414.

However, the current sEH inhibitor clinical candidates have poor blood-brain-barrier (BBB) penetration and overall poor physical properties, which negatively impacts their pharmacokinetic behavior and their utility as therapeutics. In addition, recent studies have also demonstrated that inhibitors designed to have long drug-target residence time, i.e., improving sEH inhibitors' target occupancy, potentially enhance target occupancy and consequently efficacy in the brain. Along with poor physical properties, the existing clinical candidates fail to address this parameter. Owing to the potential of sEH inhibitors as a treatment for CNS inflammatory diseases, including Alzheimer's and inflammation associated with brain injuries, there is an unmet medical need to develop sEH inhibitors with better physical properties, improved BBB penetration and enhanced drug-target residence time.

### SUMMARY

Thus, disclosed herein are potent sEH inhibitors with novel structures. These novel inhibitors have improved physical properties, BBB penetration and long drug-target residence time. The compound or pharmaceutically acceptable salt thereof disclosed herein corresponds in structure to Formula I

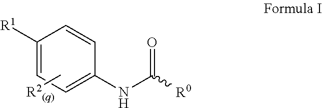

wherein the R⁰ moiety corresponds in structure to the related moieties A, B, C or D below:

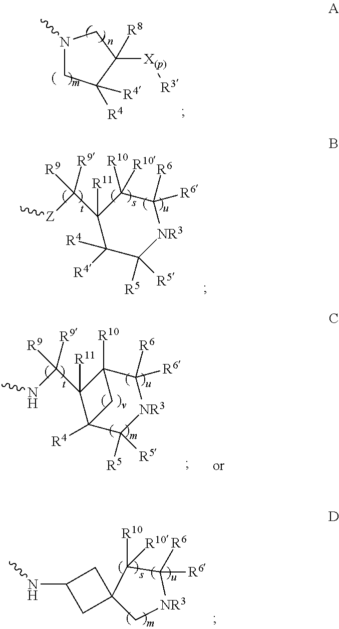

wherein R¹ is selected from the group consisting of H, halo, —OCF₃, —OCF₂H, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, —CO-fluoroalkyl, aryl, and C₁-C₆-alkyl;
R^{3′} is selected from the group consisting of —NHCO-alkyl, —NHCO-aryl, —NHCO—O-alkyl, NHCO-fluoroalkyl, —NCH₃CO—O-alkyl, —NCH₃CO-alkyl, —NH-aryl, —NH-alkyl, —NH₂, and C₁-C₆-alkoxy;
R⁴ and R^{4′} are independently selected from the group consisting of H, halo, and C₁-C₆-alkyl; or
R⁴ and R^{4′} can together form a 3-6 membered ring; or R⁴ or R^{4′} can form a 3-6 membered ring with X; or R⁴ or R^{4′} can form a 3-6 membered ring with R^{3′}; or R⁴ or R^{4′} can form a 3-6 membered ring with R⁵ or R^{5′};
X is selected from the group consisting of —C(R⁷)(R^{7′}); or X can form a 3-6 membered ring with R⁴ or R^{4′};
Z is NH or O;
R⁵ and R^{5′} are independently selected from the group consisting of H, halo, and C₁-C₆-alkyl; or
R⁵ and R^{5′} can together form a 3-6 membered ring; or R⁵ or R^{5′} can form a 3-6 membered ring with R⁴ or R^{4′};
R⁶ and R^{6′} are independently selected from the group consisting of H, halo, and C₁-C₆-alkyl; or
R⁶ or R^{6′} and can form a 3-6 membered ring with R⁴, R^{4′}, R⁵, or R^{5′}; or R⁶ and R^{6′} can together form a 3-6 membered ring;
R⁷ and R^{7′} are independently selected from the group consisting of H, C₁-C₆-alkyl, and substituted C₁-C₆-alkyl; or R⁷ and R^{7′} can together form a 3-6 membered ring;
R⁸ is H, halo, C₁-C₆-alkyl, C₁-C₆-alkoxy; or R⁸ can form a 3-6 membered ring with X; or R⁸ can form a 4-6 membered ring with X and R^{3′};
R⁹ and R^{9′} are independently H, C₁-C₆-alkyl, and aryl;
R¹⁰ and R^{10′} are independently selected from the group consisting of H, halo, and C₁-C₆-alkyl or
R¹⁰ or R^{10′} and can form a 3-6 membered ring with R⁴, R^{4′}, R⁵, or R^{5′}; or R¹⁰ or R^{10′} can together form a 3-6 membered ring;
R¹¹ is selected from the group consisting of H, halo, C₁-C₆-alkyl, and C₁-C₆-alkoxy;
m is zero, 1 or 2; n is zero, 1 or 2; p is zero, 1, 2, or 3; q is zero, 1 or 2; s is zero or 1; t is zero or 1; u is zero or 1; and v is zero, 1, or 2; and
wherein the compound is not 1-trifluoromethoxyphenyl-3-(1-propionylpiperidin-4-yl) urea (TPPU).

The sEH inhibitors disclosed herein may be used to treat CNS diseases, such as Alzheimer's disease and brain injury inflammation. Pharmaceutical compositions and kits comprising one or more if the sEH inhibitors are also disclosed herein.

### BRIEF DESCRIPTION OF THE DRAWINGS

FIG. 1 is Table 1 listing various properties and IC₅₀ values of compounds disclosed herein. See Example 106.

FIG. 2 is Table 2 listing various properties and IC₅₀ values of further compounds disclosed herein. See Example 106.

FIG. 3 is Table 3 listing various properties and IC₅₀ values of further compounds disclosed herein. See Example 106.

FIG. 4 is Table 4 listing various properties and IC₅₀ values of further compounds disclosed herein. See Example 106.

FIG. 5 is graph showing blood concentration of compounds disclosed herein. See Example 107.

FIG. 6. is graph showing blood concentration of further compounds disclosed herein. See Example 107.

FIG. 7 is a graph showing brain-to-blood ratios of compounds disclosed herein. See Example 107.

FIG. 8 is a graph showing brain-to-plasma ratios of compounds disclosed herein. See Example 107.

### DETAILED DESCRIPTION

### A. Definitions

“Alkyl” includes saturated aliphatic hydrocarbyl groups. The hydrocarbon chain may be either straight-chained or branched. Examples of “alkyl” include those with 1-6 carbon atoms (“C₁₋₆ alkyl”), those with 1-5 carbon atoms (“C₁₋₅ alkyl”), 1-4 carbon atoms (“C₁₋₄ alkyl”), or only 1-3 carbon atoms (“C₁₋₃ alkyl”). This term is exemplified by groups such as methyl, ethyl, n-propyl, isopropyl, n-butyl, sec-butyl, isobutyl, tert-butyl, t-amyl, and the like. Any numbers of C atoms in alkyls or other groups may be indicated herein in brackets or without brackets.

“Alkyloxy” and “alkoxy”, as used interchangeably herein (together alk(yl)oxy), include the group —OR wherein R is “alkyl” as defined and exemplified further herein. Particular alk(yl)oxy groups include, by way of example, meth(yl)oxy, eth(yl)oxy, n-prop(yl)oxy, isoprop(yl)oxy, n-but(yl)oxy, tert-but(yl)oxy, sec-but(yl)oxy, isobut(yl)oxy, n-pent(yl)oxy, 1,2-dimethylbut(yl)oxy, and the like.

The terms “BOC” or “Boc”, as used interchangeably herein, refer to a tert-butyloxycarbonyl moiety or —CO—O—C(CH₃)₃.

“Halogen” or “Halo” includes fluoro, chloro, bromine, and iodine atoms.

The term “haloalkyl” as used herein refers to an “alkyl” as described herein (and wherein the numbers indicate the numbers of C-atoms in the alkyl part), which is substituted with one or more halogen atoms. Representative examples of “halo(C₁₋₃)alkyl” groups include, but are not limited to —CF₃, —CCl₃, —CFCl₂, —CH₂CH₂CF₃ and —CH₂CF₃.

The term “fluorinated” refers to a group wherein one or more hydrogens are replaced with fluoros. For example, an alkyl or alkoxy group, respectively, which is said to be unsubstituted or fluorinated comprises a “fluoroalkyl” or “fluoroalk(yl)oxy”, respectively, as defined herein. Likewise, a fluorinated alkoxyalkyl group comprises the groups fluoroalkoxyalkyl and alkoxyfluoroalkyl.

The term “fluoroalkyl” as used refers to an “alkyl” as described herein, which is substituted with one or more fluoro atoms. Representative examples of fluoro(C₁₋₃)alkyl groups include, but are not limited to —CF₃, —CH₂CHF₂ and —CH₂CF₃. Preferred “fluoroalkyl” groups are those wherein terminal methyl groups are substituted with one or more fluoro atoms; hence, a particularly preferred monofluoroethyl group is —CH₂CH₂F, a particularly preferred difluoroethyl group is the group —CH₂CHF₂, a particularly preferred trifluoroethyl group is the group —CH₂CF₃, a particularly preferred monofluoropropyl group is —CH₂CH₂CH₂F, a particularly preferred difluoropropyl is —CH₂CH₂CHF₂, and, a particularly preferred trifluoropropyl group is —CH₂CH₂CF₃.

The term “alkylcarbonyl” refers to the group —C(═O)-alkyl, wherein alkyl is as defined herein. Typical examples are C₁₋₆ alkylcarbonyl and C₁₋₃ alkylcarbonyl, and in particular acetyl (—C(═O)CH₃).

A “carbocyclyl” may be a single ring structure, which typically contains from 3 to 7 ring atoms, more typically from 3 to 6 ring atoms, and even more typically 5 to 6 ring atoms. Examples of such single-ring carbocyclyls include cyclopropyl (cyclopropanyl), cyclobutyl (cyclobutanyl), cyclopentyl (cyclopentanyl), cyclopentenyl, cyclopentadienyl, cyclohexyl (cyclohexanyl), cyclohexenyl, cyclohexadienyl, and phenyl. A carbocyclyl may alternatively be polycyclic (i.e., may contain more than one ring). Examples of polycyclic carbocyclyls include bridged, fused, and spirocyclic carbocyclyls. In a spirocyclic carbocyclyl, one atom is common to two different rings. An example of a spirocyclic carbocyclyl is spiropentanyl. In a bridged carbocyclyl, the rings share at least two common non-adjacent atoms. Examples of bridged carbocyclyls include bicyclo[2.2.1]heptanyl, bicyclo[2.2.1]hept-2-enyl, and adamantanyl. In a fused-ring carbocyclyl system, two or more rings may be fused together, such that two rings share one common bond. Examples of two- or three-fused ring carbocyclyls include naphthalenyl, tetrahydronaphthalenyl (tetralinyl), indenyl, indanyl (dihydroindenyl), anthracenyl, phenanthrenyl, and decalinyl.

The term “cycloalkyl” (alone or in combination with another term(s)) means a saturated cyclic hydrocarbyl substituent containing from 3 to 14 carbon ring atoms. A cycloalkyl may be a single carbon ring, which typically contains from 3 to 7 carbon ring atoms and more typically from 3 to 6 ring atoms. Examples of single-ring cycloalkyls include cyclopropyl, cyclobutyl, cyclopentyl, and cyclohexyl. A cycloalkyl may alternatively be polycyclic or contain more than one ring. Examples of polycyclic cycloalkyls include bridged, fused, and spirocyclic carbocyclyls.

A heterocyclyl may be a single ring, which typically contains from 3 to 7 ring atoms, more typically from 3 to 6 ring atoms, and even more typically 5 to 6 ring atoms. Examples of single-ring heterocyclyls include furanyl, dihydrofuranyl, tetrahydrofuranyl, thiophenyl (thiofuranyl), dihydrothiophenyl, tetrahydrothiophenyl, pyrrolyl, pyrrolinyl, pyrrolidinyl, imidazolyl, imidazolinyl, imidazolidinyl, pyrazolyl, pyrazolinyl, pyrazolidinyl, triazolyl, tetrazolyl, oxazolyl, oxazolidinyl, isoxazolidinyl, isoxazolyl, thiazolyl, isothiazolyl, thiazolinyl, isothiazolinyl, thiazolidinyl, isothiazolidinyl, thiodiazolyl, oxadiazolyl (including 1,2,3-oxadiazolyl, 1,2,4-oxadiazolyl, 1,2,5-oxadiazolyl (furazanyl), or 1,3,4-oxadiazolyl), oxatriazolyl (including 1,2,3,4-oxatriazolyl or 1,2,3,5-oxatriazolyl), dioxazolyl (including 1,2,3-dioxazolyl, 1,2,4-dioxazolyl, 1,3,2-dioxazolyl, or 1,3,4-dioxazolyl), oxathiazolyl, oxathiolyl, oxathiolanyl, pyranyl, dihydropyranyl, thiopyranyl, tetrahydrothiopyranyl, pyridinyl (azinyl), piperidinyl, diazinyl (including pyridazinyl (1,2-diazinyl), pyrimidinyl (1,3-diazinyl), or pyrazinyl (1,4-diazinyl)), piperazinyl, triazinyl (including 1,3,5-triazinyl, 1,2,4-triazinyl, and 1,2,3-triazinyl)), oxazinyl (including 1,2-oxazinyl, 1,3-oxazinyl, or 1,4-oxazinyl)), oxathiazinyl (including 1,2,3-oxathiazinyl, 1,2,4-oxathiazinyl, 1,2,5-oxathiazinyl, or 1,2,6-oxathiazinyl)), oxadiazinyl (including 1,2,3-oxadiazinyl, 1,2,4-oxadiazinyl, 1,4,2-oxadiazinyl, or 1,3,5-oxadiazinyl)), morpholinyl, azepinyl, oxepinyl, thiepinyl, and diazepinyl.

A heterocyclyl may alternatively be polycyclic (i.e., may contain more than one ring). Examples of polycyclic heterocyclyls include bridged, fused, and spirocyclic heterocyclyls. In a spirocyclic heterocyclyl, one atom is common to two different rings. In a bridged heterocyclyl, the rings share at least two common non-adjacent atoms. In a fused-ring heterocyclyl, two or more rings may be fused together, such that two rings share one common bond. Examples of fused ring heterocyclyls containing two or three rings include indolizinyl, pyranopyrrolyl, 4H-quinolizinyl, purinyl, naphthyridinyl, pyridopyridinyl (including pyrido[3,4-b]-pyridinyl, pyrido[3,2-b]-pyridinyl, or pyrido[4,3-b]-pyridinyl), and pteridinyl. Other examples of fused-ring heterocyclyls include benzo-fused heterocyclyls, such as indolyl, isoindolyl (isobenzazolyl, pseudoisoindolyl), indoleninyl (pseudoindolyl), isoindazolyl (benzpyrazolyl), benzazinyl (including quinolinyl (1-benzazinyl) or isoquinolinyl (2-benzazinyl)), phthalazinyl, quinoxalinyl, quinazolinyl, benzodiazinyl (including cinnolinyl (1,2-benzodiazinyl) or quinazolinyl (1,3-benzodiazinyl)), benzopyranyl (including chromanyl or isochromanyl), benzoxazinyl (including 1,3,2-benzoxazinyl, 1,4,2-benzoxazinyl, 2,3,1-benzoxazinyl, or 3,1,4-benzoxazinyl), and benzisoxazinyl (including 1,2-benzisoxazinyl or 1,4-benzisoxazinyl).

The term “aryl” (alone or in combination with another term(s)) means an aromatic carbocyclyl containing from 6 to 14 carbon ring atoms. Examples of aryls include phenyl, naphthalenyl, and indenyl.

The term “heteroaryl” as used herein refers to monovalent aromatic groups containing at least five ring-forming atoms derived from a single ring with e.g. up to six atoms (e.g. “C₅₋₆ heteroaryl”) or multiple condensed rings with e.g. up to 10 ring forming atoms (e.g. “C₅₋₁₀ heteroaryl”), wherein one or more carbon atoms have been replaced by one or more heteroatoms preferably selected from oxygen, sulphur and nitrogen. Suitable heteroaryl groups include furyl, benzofuryl, thienyl, benzothienyl, thieno[2,3-c]pyrazolyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, pyrazolo[3,4-d]pyrimidinyl, indazolyl, 4,5,6,7-tetrahydroindazolyl, oxazolyl, benzoxazolyl, isoxazolyl, thiazolyl, benzothiazolyl, benzoxadiazolyl, benzoselenathiazolyl, isothiazolyl, imidazolyl, benzimidazolyl, imidazo[2, 1-b]thiazolyl, imidazo[1,2-a]pyridinyl, imidazo[4,5-b]pyridinyl, purinyl, imidazo[1,2-a]pyrimidinyl, imidazo[1,2-a]pyrazinyl, oxadiazolyl, thiadiazolyl, triazolyl, [1,2,4]triazolo[1,5-a]-5-pyrimidinyl, benzotriazolyl, tetrazolyl, pyridinyl, quinolinyl, isoquinolinyl, pyridazinyl, cinnolinyl, phthalazinyl, pyrimidinyl, quinazolinyl, pyrazinyl, quinoxalinyl, pteridinyl, and triazinyl. As indicated above, the term “C₅₋₆ heteroaryl” refers to a heteroaryl with 5- or 6-ring forming atoms although some of the ring forming atoms are no carbon atoms but heteroatoms.

“Pharmaceutically acceptable carrier” refers to a diluent, adjuvant, excipient, or carrier, or other ingredient with which a compound of the invention is administered and which a person of skilled in the art would understand to be pharmaceutically acceptable but is typically not biodynamically active.

The compounds provided herein are useful in the prevention and/or treatment of certain diseases or disorders in animals, in particular in humans, as described herein.

“Preventing” or “prevention” refers to a reduction in risk of acquiring a disease or disorder (i. e., causing at least one of the clinical symptoms of the disease not to develop in a subject, in particular a human subject, that may be exposed to or predisposed to the disease but does not yet experience or display symptoms of the disease).

“Treating” or “treatment” of any disease or disorder includes, in one embodiment, to improve the disease or disorder (i. e., arresting or reducing the development of the disease or at least reducing one of the clinical symptoms of the disease). In another embodiment “treating” or “treatment” refers to improve at least one physical parameter, which may or may not be discernible by the subject, in particular a human subject, but which is based on or associated with the disease or disorder to be treated. In yet another embodiment, “treating” or “treatment” refers to modulating the disease or disorder, either physically (e. g. stabilization of a discernible on non-discernible symptom), physiologically (e. g. stabilization of a physiological parameter), or both. In yet another embodiment, “treating” or “treatment” refers to delaying the onset or progression of the disease or disorder. Accordingly, “treating” or “treatment” includes any causal treatment of the underlying disease or disorder (i.e. disease modification), as well as any treatment of signs and symptoms of the disease or disorder (whether with or without disease modification), as well as any alleviation or amelioration of the disease or disorder, or its signs and symptoms.

The terms “disease(s)” and “disorder(s)” are used largely interchangeably herein.

The term “animal(s)” and “subject(s)” includes humans. The terms “human,” “patient” and “human subject” are used interchangeably herein.

### B. Compounds

A compound corresponding in structure to Formula I:

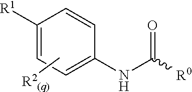

or a pharmaceutically acceptable salt, solvate, isotope or co-crystal thereof, is disclosed herein.

The R¹ substituent may be hydrogen; halo such as F, Br, Cl; haloalkyl such as —CF₃, —CF(CF₃)₂, —CHF₂; haloalkoxy such as —OCF₃, —OCF₂H, or —OCF₂; C₁-C₆-alkyl (e.g., C₁-C₅-alkyl C₁-C₄-alkyl, C₁-C₃-alkyl, C₁-C₂-alkyl, or methyl); or —SF₅.

R², if present, may be halo, such as F, Br or C₁; C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy). In some embodiments, R² may be present one or more times. This is represented by the variable “q”, where “q” may be zero, 1, 2 or 3; or “q” may be zero, 1, or 2; or “q” may be zero or 1.

In any embodiment, the compound corresponding in structure to Formula I may not be 1-trifluoromethoxyphenyl-3-(1-propionylpiperidin-4-yl) urea (TPPU).

The R^{°} substituent may be a structure corresponding to Formulas A, B, C or D (also referred to as Formula IA, IB, IC and ID compounds).

### Formula IA Compounds

In some embodiments, R^{°} is represented by Formula A

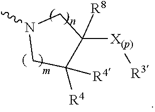

In Formula A, the variables “m” and “n” each independently may be zero, 1 or 2. In particular embodiments, “m” and “n” independently are 1 or 2. In further embodiments, “m” is zero and “n” is 1; or “m” and “n” are each 1; or “m” is 1 and “n” is 2; or “m” is 2 and “n” is 1; or “m” is 1 and “n” is zero.

The variable R^{3′} may be —NHCO-alkyl, —NHCO-aryl, —NHCO-heteroaryl, —NHCO—O-alkyl, —NHCO-fluoroalkyl, —NCH₃CO—O-alkyl, —NCH₃CO-alkyl, —NH-alkyl, —NH-aryl, —NH— heteroaryl, —NH₂, or C₁-C₆-alkoxy (e.g., C₁-C₅-alkoxy, C₁-C₄-alkoxy, C₁-C₃-alkoxy, C₁-C₂-alkoxy, or methoxy). In particular embodiments, R^{3′} is —NHCO-fluoroalkyl, —NHCO-alkyl or —NHCO—O-alkyl or R^{3′} is —NHCO-alkyl or —NHCO—O-alkyl. For example, R^{3′} may be —NHCO—CF₂CF₃, —NHCO—O—C(CH₃)₃ (also referred to as “NHBoc”), —NCH₃CO—O—C(CH₃)₃, —NHCO—CH₂CH₃, —NHCO—C(CH₃)₃, —NHCO—CH(CH₃)₂, or —NHCO—CH₂CH(CH₃)₂.

The variables R⁴ and R^{4′} may independently be H; halo such as C₁, F, Br; or C₁-C₆-alkyl, e.g., C₁-C₅-alkyl C₁-C₄-alkyl, C₁-C₃-alkyl, C₁-C₂-alkyl, or methyl; or R⁴ and R^{4′} may together form a 3-6 membered ring (e.g., 3, 4, 5, or 6 membered ring) such as a carbocyclyl (either saturated or unsaturated as defined herein) or a heterocyclyl (saturated, partially saturated or completely unsaturated as defined herein); or R⁴ or R^{4′} may form a 3-6 membered ring (e.g., 3, 4, 5, or 6 membered ring) with the variable X if X is present; or R⁴ or R^{4′} can form a 3-6 membered ring (e.g., 3, 4, 5, or 6 membered ring) with R^{3′}.

The variable X, if present, may be —C(R⁷)(R^{7′}); or X may form a 3-6 membered ring with R⁴ or R^{4′} as discussed above. X may be absent or may be present one time. This is represented by the variable “p”, where “p” may be zero, 1, 2, or 3; or “p” may be zero, 1, or 2; or “p” may be zero or 1.

In some embodiments, where X is present and is —C(R⁷)(R^{7′}), R⁷ and R^{7′} may independently be H, C₁-C₆-alkoxy (e.g., C₁-C₅-alkoxy, C₁-C₄-alkoxy, C₁-C₃-alkoxy, C₁-C₂-alkoxy, or methoxy), C₁-C₆-alkyl (e.g., C₁-C₅-alkyl C₁-C₄-alkyl, C₁-C₃-alkyl, C₁-C₂-alkyl, or methyl), or substituted C₁-C₆-alkyl; or R⁷ and R^{7′} may together form a 3-6 membered ring (e.g., 3, 4, 5, or 6 membered ring) such as a carbocyclyl (either saturated or unsaturated as defined herein) or a heterocyclyl (saturated, partially saturated or completely unsaturated as defined herein).

Lastly, in Formula A R⁸ may be H; halo such as F, Cl, Br; C₁-C₆-alkyl (e.g., C₁-C₅-alkyl C₁-C₄-alkyl, C₁-C₃-alkyl, C₁-C₂-alkyl, or methyl); C₁-C₆-alkoxy (e.g., C₁-C₅-alkoxy, C₁-C₄-alkoxy, C₁-C₃-alkoxy, C₁-C₂-alkoxy, or methoxy); or R⁸ may form a 3-6 membered ring (e.g., 3, 4, 5, or 6 membered ring) with X; or R⁸ may form a 4-6 membered ring (e.g., 4, 5, or 6 membered ring) with X and R^{3′}. In a particular embodiment, R⁸ is H. In the ring embodiments for R⁸, the ring may be a carbocyclyl (either saturated or unsaturated as defined herein) or a heterocyclyl (saturated, partially saturated or completely unsaturated as defined herein).

It is understood herein, that when any of moieties described herein form a ring with another moiety described herein, each moiety may lose a hydrogen atom to form a ring. For example, when R⁴ or R^{4′} form a 3-6 membered ring with R^{3′}, R⁴ or R^{4′} and R^{3′} may each loss a hydrogen atom to form the 3-6 membered ring.

### Formula IB Compounds

In further embodiments, R⁰ is represented by Formula B

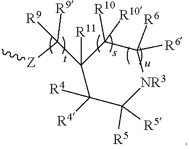

In Formula B, Z may be NH or O and the R³ variable may be —CO-alkyl, —CO-aryl, —CO-heteroaryl, —CO—O-alkyl, —CO—O-alkylaryl, —CO—O-alkylheteroaryl, —CO-fluoroalkyl, aryl, or C₁-C₆-alkyl (e.g., C₁-C₅-alkyl C₁-C₄-alkyl, C₁-C₃-alkyl, C₁-C₂-alkyl, or methyl). In particular embodiments, R³ is —CO-fluoroalkyl, —CO-alkyl or —CO—O-alkyl or R³ is —CO-alkyl or —CO—O-alkyl. For example, R³ may be —CO—CH₂CH₂F, —CO—O—C(CH₃)₃, —CO—CH₂CH₃, —CO—C(CH₃)₃, —CO—CH(CH₃)₂, or —CO—CH₂CH(CH₃)₂.

Similar to Formula A, the variables R⁴ and R^{4′} may independently be H; halo such as C₁, F, Br; C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy); or R⁴ and R^{4′} may together form a 3-6 membered ring (e.g., 3, 4, 5, or 6 membered ring); or R⁴ or R^{4′} may form a 3-6 membered ring (e.g., 3, 4, 5, or 6 membered ring) with R⁵ or R^{5′}. In embodiments where R⁴ and/or R^{4′} form a 3-6 membered ring, the 3-6 membered ring may be a carbocyclyl (either saturated or unsaturated as defined herein) or a heterocyclyl (saturated, partially saturated or completely unsaturated as defined herein).

Likewise, the variables R⁵ and R^{5′} may independently be H; halo such as C₁, F, Br; C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy); or R⁵ and R^{5′} may together form a 3-6 membered ring (e.g., 3, 4, 5, or 6 membered ring); or R⁵ or R^{5′} may form a 3-6 membered ring (e.g., 3, 4, 5, or 6 membered ring) with R⁴ or R^{4′}. In embodiments where R⁵ and/or R^{5′} form a 3-6 membered ring, the 3-6 membered ring may be a carbocyclyl (either saturated or unsaturated as defined herein) or a heterocyclyl (saturated, partially saturated or completely unsaturated as defined herein).

The variable “u” may be zero or 1. When “u” is 1, then R⁶ and R^{6′} are present and may be H, halo, C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy); or R⁶ or R^{6′} may form a 3-6 membered ring (e.g., 3, 4, 5, or 6 membered ring) with R⁴, R^{4′}, R⁵, or R^{5′}; or R⁶ and R^{6′} may together form a 3-6 membered ring (e.g., 3, 4, 5, or 6 membered ring). In a particular embodiment, “u” is 1 and R⁶ and/or R^{6′} are independently H or C₁-C₆-alkyl. In embodiments where R⁶ and/or R^{6′} form a 3-6 membered ring, the 3-6 membered ring may be a carbocyclyl (either saturated or unsaturated as defined herein) or a heterocyclyl (saturated, partially saturated or completely unsaturated as defined herein).

The variable “t” may be zero or 1. When “t” is 1, then R⁹ and R^{9′} are present and are independently H, C₁-C₆-alkyl (e.g., C₁-C₅-alkyl C₁-C₄-alkyl, C₁-C₃-alkyl, C₁-C₂-alkyl, or methyl), C₁-C₆-alkoxy (e.g., C₁-C₅-alkoxy, C₁-C₄-alkoxy, C₁-C₃-alkoxy, C₁-C₂-alkoxy, or methoxy), aryl, or heteroaryl. In a particular embodiment, “t” is 1 and R⁹ and R^{9′} are independently H or C₁-C₆-alkyl.

The variable “s” may be zero or 1. When “s” is 1, then R¹⁰ and R^{10′} are present and may be H, halo, C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy); or R¹⁰ or R^{10′} may form a 3-6 membered ring (e.g., 3, 4, 5, or 6 membered ring) with R⁴, R^{4′}, R⁵, or R^{5′}; or R¹⁰ and R^{10′} may together form a 3-6 membered ring (e.g., 3, 4, 5, or 6 membered ring). In a particular embodiment, “s” is 1 and R¹⁰ and/or R^{10′} are independently H or C₁-C₆-alkyl. In embodiments where R¹⁰ and/or R^{10′} form a 3-6 membered ring, the 3-6 membered ring may be a carbocyclyl (either saturated or unsaturated as defined herein) or a heterocyclyl (saturated, partially saturated or completely unsaturated as defined herein).

The variable R¹¹ may be H, halo, C₁-C₆-alkyl (e.g., C₁-C₅-alkyl C₁-C₄-alkyl, C₁-C₃-alkyl, C₁-C₂-alkyl, or methyl); or C₁-C₆-alkoxy (e.g., C₁-C₅-alkoxy, C₁-C₄-alkoxy, C₁-C₃-alkoxy, C₁-C₂-alkoxy, or methoxy). In a particular embodiment, R¹¹ is H or C₁-C₆-alkyl.

In any embodiment, when R⁰ is B, the compound corresponding in structure to Formula I may not be 1-trifluoromethoxyphenyl-3-(1-propionylpiperidin-4-yl) urea (TPPU).

### Formula IC Compounds

In further embodiments, R⁰ is represented by the Formula C

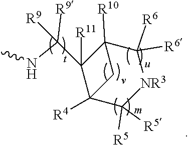

Formula C is similar to Formula B, except an additional 3-5 membered ring is present when the variable “v” is zero, 1 or 2. The variables R³, R⁴, R⁵, R^{5′}, R⁶, R^{6′}, R⁹, R^{9′}, R¹⁰, R¹¹, t, and u are the same as described above for Formula B. The variable “m” may be zero, 1, or 2; or “m” may be zero or 1; or “m” may be zero. When “m” is 1 or 2, then R⁵ and R^{5′} are present.

### Formula ID Compounds

Lastly, in further embodiments R⁰ is represented by Formula D

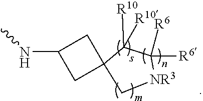

Formula D is also similar to Formula B, except a 4-membered ring is present next to the —NH group as shown above. In Formula D, R³, R⁶, R^{6′}, R¹⁰, R^{10′}, s and u are the same as described above for Formula B. The variable “m” may be zero, 1, or 2; or “m” may be zero or 1; or “m” may be zero.

In any embodiment described above, an alkyl may be a C₁-C₁₂-alkyl, C₁-C₁₀-alkyl, C₁-C₈-alkyl, C₁-C₆-alkyl, C₁-C₅-alkyl, C₁-C₄-alkyl, C₁-C₃-alkyl, C₁-C₂-alkyl, C₂-C₁₂-alkyl, C₂-C₁₀-alkyl, C₂-C₈-alkyl, C₂-C₆-alkyl, C₂-C₅-alkyl, C₂-C₄-alkyl, C₂-C₃-alkyl, C₃-C₁₂-alkyl, C₃-C₁₀-alkyl, C₃-C₈-alkyl, C₃-C₆-alkyl, C₃-C₅-alkyl, C₃-C₄-alkyl, or methyl. For example, —CO-alkyl may be —CO—(C₁-C₆)alkyl, —CO—(C₁-C₅)alkyl, —CO—(C₁-C₄)alkyl, —CO—(C₁-C₃)alkyl, —CO—(C₁-C₂)alkyl, CO—(C₂-C₆)alkyl, —CO—(C₂-C₅)alkyl, —CO—(C₂-C₄)alkyl, —CO—(C₂-C₃)alkyl, CO—(C₃-C₆)alkyl, —CO—(C₃-C₅)alkyl, —CO—(C₃-C₄)alkyl, or —CO-methyl, and —CO—O-alkyl may be —CO—O—(C₁-C₆)alkyl, —CO—O—(C₁-C₅)alkyl, —CO—O—(C₁-C₄)alkyl, —CO—O—(C₁-C₃)alkyl, —CO—O—(C₁-C₂)alkyl, —CO—O—(C₂-C₆)alkyl, —CO—O—(C₂-C₅)alkyl, —CO—O—(C₂-C₄)alkyl, —CO—O—(C₂-C₃)alkyl, —CO—O—(C₃-C₆)alkyl, —CO—O—(C₃-C₅)alkyl, —CO—O—(C₃-C₄)alkyl, or —CO—O-methyl, and so on.

In a particular embodiment, the compound may be one of the following species:

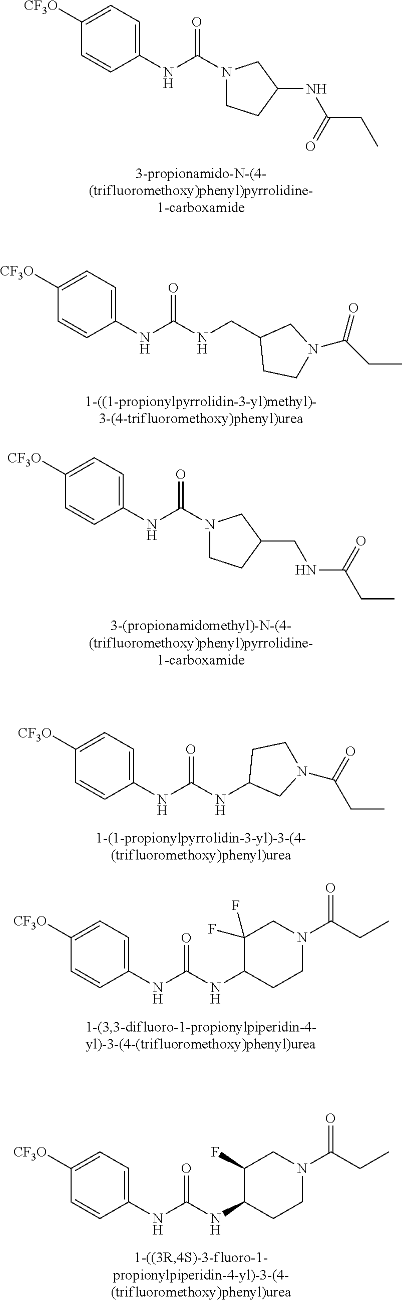

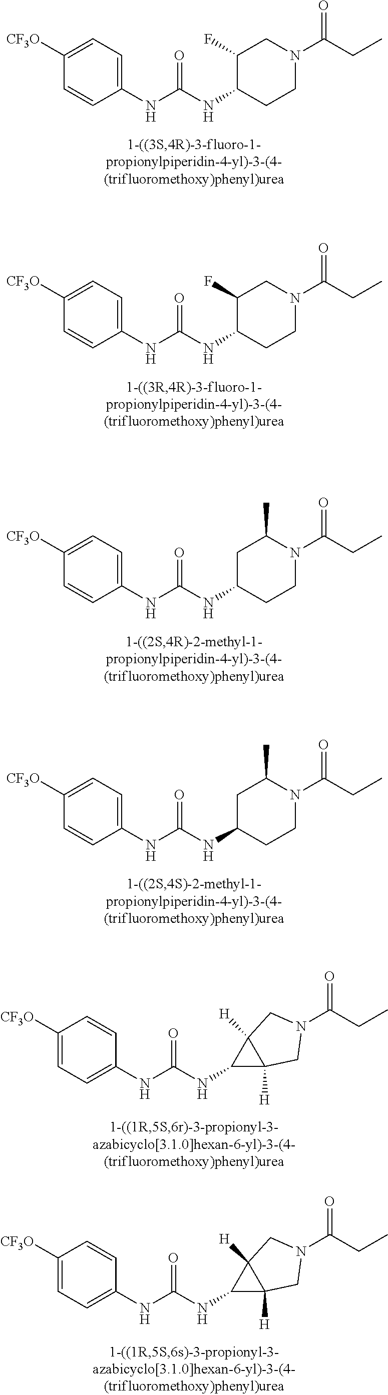

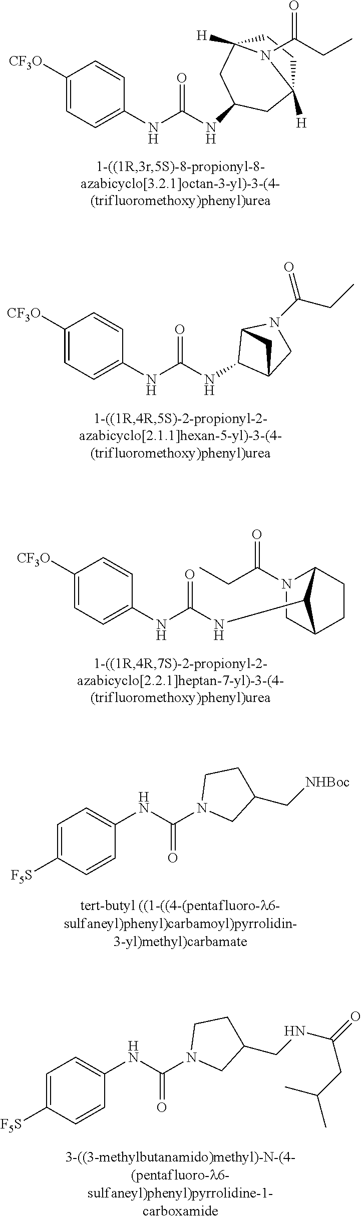

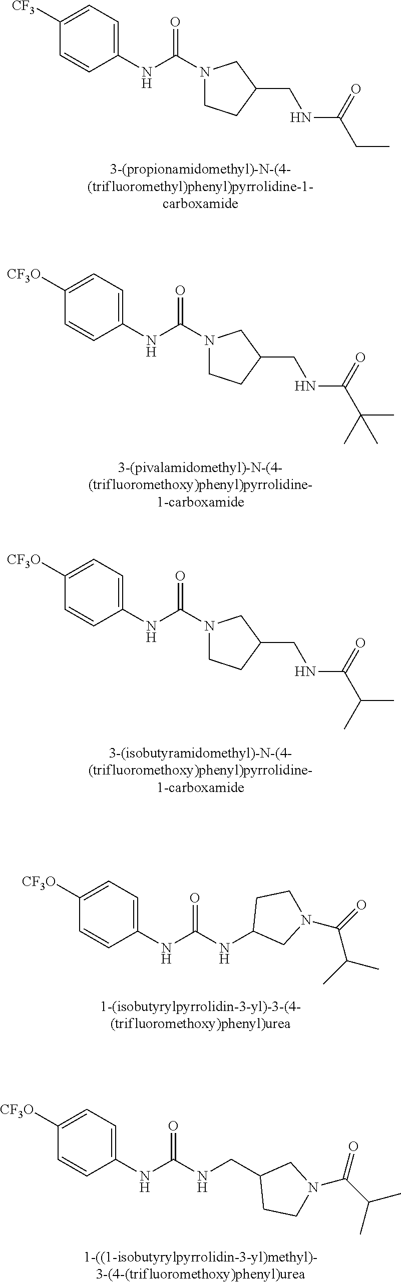

### C. Salts/Polymorphs/Solvates/Co-Crystals/Isotopes

Pharmaceutically acceptable salts of the compounds disclosed herein are also contemplated for use. The term “pharmaceutically acceptable salts” relates to any salts that the compounds of the present invention may form and which are suitable for administration to subjects, in particular human subjects. Such salts include but are not limited to acid addition salts, formed either with inorganic acids such as hydrochloric acid, hydrobromic acid, sulfuric acid, nitric acid, phosphoric acid, and the like, or formed with organic acids such as acetic acid, trifluoroacetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]oct-2-ene-1-6arboxyic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. Other salts include 2,2-dichloroacetate, adipate, alginate, ascorbate, aspartate, 2-acetamidobenzoate, caproate, caprate, camphorate, cyclamate, laurylsulfate, edisilate, esylate, isethionate, formate, galactarate, gentisate, gluceptate, glucuronate, oxoglutarate, hippurate, lactobionate, napadisilate, xinafoate, nicotinate, oleate, orotate, oxalate, palmitate, embonate, pidolate, p-aminosalicylate, sebacate, tannate, rhodanide, undecylenate, and the like; or salts formed when an acidic proton present in the parent compound is replaced, such as with ammonia, arginine, benethamine, benzathine, calcium, choline, deanol, diethanolamine, diethylamine, ethanolamine, ethylendiamine, meglumine, glycine, hydrabamine, imidazole, lysine, magnesium, hydroxyethylmorpholine, piperazine, potassium, epolamine, sodium, trolamine, tromethamine or zinc. For example, a pharmaceutically acceptable salt of the compounds disclosed herein may be a salt of an acid, such as trifluoroacetic acid.

The compound of the present invention may also exist in different crystal forms, i.e. as polymorphs, all of which are encompassed by the present invention.

The present invention also includes within its scope solvates of the compounds as defined herein. “Solvates” are crystals formed by an active compound and a second component (solvent) which, in isolated form, is liquid at room temperature. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds herein may be formed with water, in which case they will be hydrates.

The present invention also includes co-crystals within its scope. The term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity. Examples of co-crystal formers, which may be present in the co-crystal alongside the active pharmaceutical ingredient, include L-ascorbic acid, citric acid, glutaric acid, cinnamic acid, mandelic acid, urea and nicotinamide.

The present invention also includes all suitable isotopic variations of a compound of the invention. An isotopic variation of a compound of the invention is defined as one in which at least one atom is replaced by an atom having the same atomic number but an atomic mass different from the atomic mass usually found in nature with the more abundant isotope(s) being preferred. Examples of isotopes that can be incorporated into compounds of the invention include isotopes of hydrogen, carbon, nitrogen, oxygen, sulphur, fluoro and chloro such as ²H, ³H, ¹¹C, ¹³C, ¹⁴C, ¹⁵N, ¹⁷O, ¹⁸O, ³⁵S, ¹⁸F, and ³⁶Cl, respectively. It is to be understood that for any isotope that is present in measurable amounts in nature, like e.g. deuterium, the amount of the corresponding radionuclide that may be introduced into the compounds of the present invention to modulate its properties, will advantageously exceed its natural abundance in nature. Hence, for example, the rate of deuterium introduced in the deuterated compounds of the present invention is typically higher than the amount of deuterium to be naturally expected in said compound. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life, reduced dosage requirements and hence may be preferred in some circumstances. Isotopic variations of the compounds of the invention can generally be prepared by conventional procedures using appropriate isotopic variations of suitable reagents.

Also part of the invention are those compounds wherein at least one atom has been replaced by a radioisotope (radionuclide) of the same or a different atom that can be used in vivo imaging techniques such as single-photon emission computed tomography (SPECT), positron emission tomography (PET), magnetic resonance spectroscopy (MRS) or magnetic resonance imaging (MRI).

### D. Methods of Use

A compound or pharmaceutically acceptable salt as described herein can be used as a medicine, in particular for use as a medicine for preventing, reducing the occurrence of, delaying the occurrence of, or treating an epoxy-fatty acid (EpFA)-associated disease, i.e. a disease which is associated with EpFA activity. Without being bound by theory, inhibiting sEH sustains endogenous EpFAs to attain their biological effects. Diseases/disorders associated with EpFA include, for example, inflammatory diseases such as inflammatory bowel disease and chronic peptide ulcer; destructive bone diseases such as arthritis, osteoporosis and sepsis; cardiovascular diseases; neurodegenerative diseases such as stroke, seizure, Alzheimer's disease, Parkinson's disease, depression; and pain such as inflammatory pain and neuropathic pain.

The compounds and salts herein are particularly advantageous for these types of disorders because they have improved physical properties, blood-brain-barrier (BBB) penetration, and long drug-target residence time which solve the CNS exposure problem.

Thus, in a particular embodiment, the invention relates to any one of the compounds or salts thereof disclosed herein, for use in preventing, reducing the occurrence of, delaying the occurrence of, or treating a CNS disease and/or inflammatory disease, such as Alzheimer's disease or inflammation associated with brain injury.

In one aspect, the compound or salt of the present invention may be used in the prevention and treatment of Alzheimer's disease which comprises administering to a patient in need thereof a therapeutically effective amount of a compound or salt thereof as described herein.

In another aspect, the compound or salt of the present invention may be used in the prevention and treatment of a disorder or syndrome associated with brain tissue damage, which comprises administering to a patient in need thereof a therapeutically effective amount of a compound as described herein. A patient in need of such a treatment can be any patient who suffered brain tissue damage such as by mechanical, chemical, viral, or other trauma.

In another aspect, the compound or salt of the present invention may be used in the prevention and treatment of a spinal cord injury, perinatal encephalopathy, stroke, ischemia, or a cerebrovascular disorder, or for improving the recovery following these events.

The treatment or prevention of a CNS disease, such as Alzheimer's disease and brain injury, also includes the treatment of the signs and symptoms associated with such a disease. Hence, the compounds or salts of the present invention may also be used to treat a disorder or syndrome associated with Alzheimer's disease or brain tissue damage, such as depression, Parkinson's disease, autism, traumatic brain injury, Huntington disease, stroke, neuroinflammation, and neurolupus.

### E. Combination Therapy (Co-Therapy)

The treatments according to the present invention may comprise the administration of one of the presently disclosed compounds or salts thereof as a “stand alone” treatment of a CNS disease, in particular of a disorder such as Alzheimer's disease and inflammation associated with brain injuries. Alternatively, a compound or salt thereof disclosed herein may be administered together with other useful drugs in a combination therapy.

In a non-limiting example, a compound or salt thereof according to the present invention is combined with another medicament for treating a CNS disease, such as Alzheimer's disease, said other medicament having a different mode of action, such as e.g. an anti-inflammatory drug. Likewise, a compound or salt thereof of the present invention maybe combined with an analgesic drug if a painful condition is to be treated. Also, a compound or salt thereof of the present disclosure may be used in combination with an antidepressant to co-treat psychological effects associated with CNS diseases.

In combination therapies, the two or more active principles/agents may be provided via the same formulation or as a “kit of parts”, i.e. in separate galenic units to be used in combination. Also, the two or more active principles, including the compounds or salts of the present invention, may be administered to the patient at the same time or subsequently, e.g. in an interval therapy. The additional drug may be administered by the same mode or a different mode of administration. For example, the sEH inhibitor of the present invention may be administered orally, while the second medicament may be administered by subcutaneous injection.

### F. Pharmaceutical Compositions/Administration/Dosing

In another embodiment, the present invention relates to a pharmaceutical composition comprising a compound or salt thereof as described herein, and a pharmaceutical acceptable carrier.

For the administration as a medicinal drug, the compounds or salts thereof may be used in a pharmaceutical composition comprising a compound or salt thereof of the present disclosure, and a pharmaceutically acceptable carrier, as further defined herein. Such a pharmaceutical composition can be adapted, for example, for oral, intravenous, intramuscular, subcutaneous, nasal, rectal, buccal or transdermal administration and may comprise pharmaceutically acceptable carriers, adjuvants, diluents, stabilizers and the like.

In one embodiment, the compounds or salts of the present invention may be administered orally, e.g. in the form of a tablet, a capsule, a dragee, a powder, a granulate, or in form of a liquid or a semi-solid, including e.g. syrups, suspensions, emulsions or solutions, by way of non-limiting example.

For instance, the compounds or salts of the present invention may be dissolved in oils, propylene glycol or other solvents which are commonly used to produce an injection. Suitable examples of the carriers include, but not limited to, physiological saline, polyethylene glycol, ethanol, vegetable oils, isopropyl myristate, etc. The compounds of the present invention may be formulated into injections by dissolving, suspending or emulsifying in water-soluble solvent such as saline and 5% dextrose, or in water-insoluble solvents such as vegetable oils, synthetic fatty acid glyceride, higher fatty acid esters and propylene glycol. The formulations of the invention may include any of conventional additives such as dissolving agents, isotonic agents, suspending agents, emulsifiers, stabilizers and preservatives.

A tablet may provide an immediate release or sustained release of the compounds or salts of the present invention.

Oral formulations, such as tablets, may contain, without limitation, sustained release agents, disintegrants, fillers, lubricants, stabilizers, antioxidants, flavours, dispersion agents, electrolytes, buffers, dyes, or conservation agents. Suitable excipients and formulations are known to those skilled in the art and are disclosed in standard monographs such as like Remington (“The science and practice of pharmacy”, Lippincott, Williams & Wilkins, 2000).

Non-limiting examples of disintegrants include pregelatinised starch, sodium starch glycolate, microcrystalline cellulose, carboxymethylcellulose sodium (CMC-Na), cross-linked CMC-Na, and low-substituted hydroxypropylcellulose, as well as mixtures thereof.

Suitable fillers and binders include without limitation microcrystalline cellulose, powdered cellulose, lactose (anhydrous or monohydrate), compressible sugar, starch (e.g. corn starch or potato starch), pregelatinised starch, fructose, sucrose, dextrose, dextrans, other sugars such as mannitol, maltitol, sorbitol, lactitol and saccharose, siliconised microcrystalline cellulose, calcium hydrogen phosphate, calcium hydrogen phosphate dehydrate, dicalciumphosphate dehydrate, tricalciumphophate, calcium lactate or mixtures thereof.

Lubricants, antiadherents and/or glidants include stearic acid, magnesium stearate, calcium stearate, sodium lauryl sulphate, hydrogenated vegetable oil, hydrogenated castor oil, sodium stearyl fumarate, macrogols, glycerol dibehenate, talc, corn starch, silicon dioxide, and the like, including mixtures.

Typical sustained release agents are for example those that swell upon contact with water such as polyvinylpyrrolidone, hydroxyethylcellulose, hydroxypropylcellulose, other cellulose ethers, starch, pregelatinised starch, polymethacrylate, polyvinylacetate, microcrystalline cellulose, dextrans, and mixtures of these. Other sustained release agents may be those that can be incorporated in a functional coating, which prevents the rapid disintegration and/or release of the active ingredient from the tablet core. Examples of agents that can be used in a functional coating are e.g. acrylic resins, cellulose derivatives such as hydroxypropylmethylcellulose acetate phthalate, hydroxypropylcellulose, or ethylcellulose, vinyl acetate derivatives, polyvinyl pyrrolidone, polyvinyl acetate, shellac, methacrylate polymers or methacrylate copolymers.

A tablet can, for example, be prepared by mixing at least one compound of the present invention with at least one non-toxic pharmaceutically acceptable excipient, such as e.g. binder, filler/diluents, disintegrant agents, plastisizer, and the like, and an optional solvent (aqueous or non-aqueous), and by subsequent processing the mixture to a tablet by a process including but not limited to dry compression, dry granulation, wet granulation, spray drying, or melt extrusion. A tablet can either be uncoated, or coated by known techniques to either mask the bad taste of an unpleasant tasting drug, or delay disintegration and absorption of the active ingredient in the gastrointestinal tract.

The compounds or salts of the present invention may also be formulated for parenteral administration by injection, e.g. by bolus injection or infusion. The compositions for injection may be provided ready to use and may take such forms as suspensions, solutions, or emulsions in oily or aqueous vehicles, and may contain excipients such as suspending, stabilising, preserving and/or dispersing agents. Alternatively, the active ingredient may be in powder form for constitution with a suitable vehicle, e.g. sterile pyrogen-free water or saline, before use.

For nasal administration or administration by inhalation, the compounds or salts according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

For ophthalmic administration, the compounds or salts for use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration, the compounds or salts for use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

In one embodiment, the compounds or salts for use in the present invention may be administered transdermally. This mode of administration prevents the so-called 1^{st} pass effect of oral administration and moreover allows providing more constant plasma levels which is of particular advantage in some instances. The design of transdermal forms such as ointments or creams or other transdermal systems such as e.g. patches or electrophoretic devices is generally known from the art, see e.g. Prausnitz and Langer, Nat Biotechnology 2008, Vol 26.11 p 1261; WO 2001/47503; WO2009/000262; WO99/49852.

The preferable dose level of the compounds or salts according to the present invention depends upon a variety of factors including the condition and body weight of the patient, severity of the particular disease, dosage form, and route and period of administration, but may appropriately be chosen by those skilled in the art. In various embodiments, the compounds or salts are administered in an amount ranging from 0.001 to 10 mg/kg of body weight per day, or from 0.03 to 1 mg/kg of body weight per day. Individual doses may range from about 0.1 to 1000 mg of active ingredient per day, from about 0.2 to 750 mg/day, from about 0.3 to 500 mg/day, from 0.5 to 300 mg/day, or from 1 to 100 mg/day. Doses may be administered once a day, or several times a day, preferably with each divided portions.

### G. Kits

Another aspect of the present invention is a kit comprising a compound of Formula I or a pharmaceutically salt thereof, or a pharmaceutical composition as described herein, and instructions for its use. The kit may also contain further active agents when co-therapy is desired. If more than one active agent is present, the active agents may be separately packaged for “co-presentation” to the patient/subject in the kit.

### Examples

The following examples are merely illustrative, and do not limit this disclosure in any way. Unless described otherwise, the compounds of the present disclosure are prepared according to the below methods.

### Generic Procedure for Urea Synthesis (1):

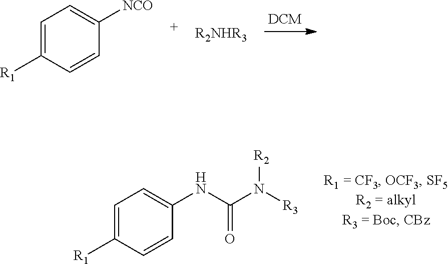

General Method A. To a 100 mL 24/40 round bottom flask was added a stir bar, dichloromethane (10.0 mL), and isocyanate (3.00 mmol, 1 eq.) under an argon atmosphere. The amine (3.17 mmol, 1.05 eq.) was dissolved in dichloromethane (1.0 mL) and added to the reaction mixture via syringe. The reaction was stirred for 19 hours, concentrated in vacuo, and purified by medium pressure chromatography (SiO₂, see individual analogs) to yield the product.

### General Method B

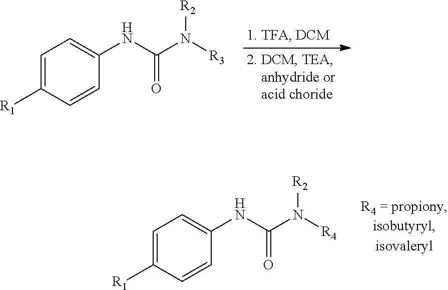

General method B. To a 100 mL 24/40 round-bottom flask was added the mono Boc-protected diamine (3.00 mmol), a stir bar and dichloromethane (15.0 mL). The reaction vessel was sealed and flushed with argon. Trifluoroacetic acid (65.0 mmol) or a 4.0 M solution of HCl in dioxane (3.0 mL, 12 mmol) was added via syringe. The mixture was stirred for 20 hours then concentrated in vacuo and dried to a constant weight under vacuum. A portion of the crude amine salt (1.9 mmol) was dissolved in dichloromethane (8.0 mL), triethylamine (8.0 mmol), and anhydride or acid chloride (4.0 mmol) added dropwise via syringe. The mixture was stirred for 18 hours at room temperature and washed with 1.0 N aqueous HCl (10.0 mL), saturated aqueous sodium bicarbonate (10.0 mL) and brine (10.0 mL). The organic layer was then dried over sodium sulfate, filtered, and concentrated in vacuo. The product was purified by normal and/or reverse chromatography (specifics indicated with each compound) to yield the product.

### Generic Procedure for Urea Synthesis (2)

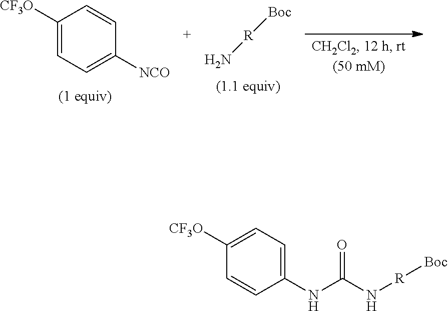

General Method C. To a 20 mL scintillation vial was added a micro stir bar, dichloromethane (9.85 mL), and isocyanate (0.492 mmol, 1.00 eq.). The amine (0.541 mmol, 1.10 eq.) was dissolved in dichloromethane to create a 50 mM reaction mixture. The reaction was stirred for 12 hours under room temperature while monitored by TLC (1:3, EtOAc:Hex) under UV light (254 nm). The reaction was quenched by adding water (5 mL). The organic layer was isolated and the aqueous layer was extracted 3 times with EtOAc (30 mL). The combined organic layer was concentrated under vacuum. The crude product was purified by silica gel chromatography using gradient conditions (EtOAc:Hex) yielding the final product.

### General Method G

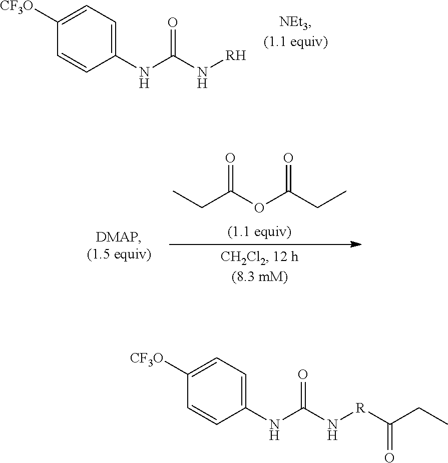

The deprotected amine was mixed with propionic anhydride (1.1 equiv), 4-dimethylaminopyridine (1.5 equiv), and triethylamine (1.1 equiv) in DCM to create a 8.3 mM solution. The reaction mixture was stirred at rt for 12 h. The reaction was monitored by TLC, (EtOAc:Hex/4:1 containing 2% MeOH). The crude product was purified by flash chromatography with (EtOAc:Hexane containing 2% MeOH) to yield the final product.

### General Method H

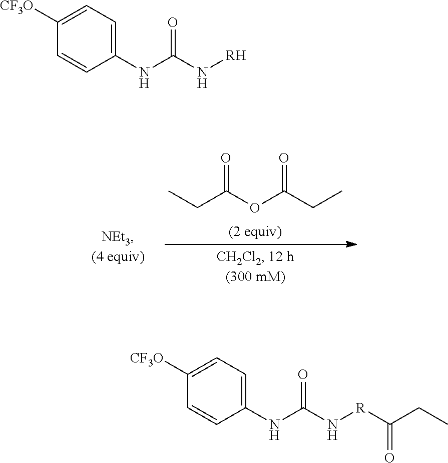

The deprotected amine was mixed with propionic anhydride (2 equiv) and triethylamine (4 equiv) to create a 300 mM solution. The resulting solution was stirred for 12 hours at rt. The reaction was monitored by TLC, (EtOAc:Hex/4:1 containing 2% MeOH). The crude product was purified by flash chromatography with (EtOAc:Hexane containing 2% MeOH) to yield the final product.

eLogP Determination:

The elogP was determined on HPLC using Shimadzu SIL-10AP equipped with C₁₈ reverse phase analytical column (C18, 4.6 mm×150 mm, 5 μm) coupled with UV detection at 230 nm. All compounds were run under isocratic conditions (MeOH:water/2:1 (v:v)) for 40 to 90 minutes. Standards with previously determined elogP were injected into the HPLC at a concentration of 16.7 μM, and each standard retention time was recorded. From the retention time, a calibration curve of elogP vs. retention time was constructed. Next, the synthesized inhibitors were injected into the HPLC at a concentration of 100 μM, and the retention time for each inhibitor was recorded. Using the calibration curve and retention times of the inhibitors, the elogP was extrapolated. Triplicate measurements were taken to ensure consistency within the data.

### Thermodynamic Solubility Determination:

1 mg of compound was added to 300 μL of PB buffer at pH 7.4 to make a suspension. The suspension shook (220 rpm) for 24 hours at 30° C. Next, the suspension was cooled to room temperature for an hour. After this, the suspensions were subjected to centrifugation for 10 minutes at 15,000 g. Supernatant from the suspension was transferred to a 1.5 mL centrifuge tube with a 0.45 μm filter and diluted 5 times by methanol. This solution was kept on ice for 15 minutes to ensure all salt precipitates out. Finally, the solution was centrifuged (15,000 g) at 4° C. for 10 minutes, and the supernatant was transferred to a new vial and stored at −20° C. before HPLC analysis. All compounds were run under isocratic conditions (MeOH:water/2:1 (v:v)) for 40 minutes. Triplicate measurements were taken to ensure consistency within the data.

### Example 1: MSU-43305/ELE-3-59. tert-Butyl N-(1-{[4-(trifluoromethoxy)phenyl]carbamoyl}pyrrolidin-3-yl)carbamate

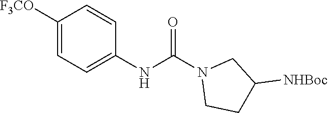 [SMILES: CC(C)(C)OC(=O)NC1CCN(C(=O)Nc2ccc(OC(F)(F)F)cc2)C1]

The product was prepared in 26% yield (0.25 g) from 4-trifluoromethoxyphenylisocyanate (0.50 g, 2.4 mmol) and carbamic acid, N-3-pyrrolidinyl-, 1,1-dimethylethyl ester (0.50 g, 2.7 mmol) by general method A. Product was purified by medium pressure chromatography (SiO₂, dichloromethane to 9/1 dichloromethane/methanol) to provide a solid. ¹H NMR (500 MHz, DMSO-d6) δ 8.31 (s, 1H), 7.65-7.57 (m, 2H), 7.25-7.16 (m, 2H), 4.00 (d, J=5.5 Hz, 1H), 3.55 (dd, J=10.5, 6.3 Hz, 1H), 3.47 (dt, J=10.2, 7.2 Hz, 1H), 3.40-3.33 (m, 1H), 3.17 (td, J=10.0, 9.6, 5.1 Hz, 1H), 2.01 (dt, J=12.5, 6.7 Hz, 1H), 1.77 (dq, J=12.8, 6.5 Hz, 1H), 1.38 (s, 9H). HRMS ESI (+) calc'd for [M+Na]=412.1460, observed=412.1491.

### Example 2: MSU-43306/ELE-3-62. tert-butyl 3-({[4-(trifluoromethoxy)phenyl]carbamoyl}amino)pyrrolidine-1-carboxylate

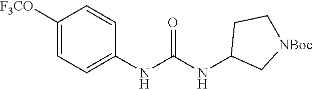 [SMILES: CC(C)(C)OC(=O)N1CCC(NC(=O)Nc2ccc(OC(F)(F)F)cc2)C1]

The product was prepared in 92% yield (0.88 g) from 4-trifluoromethoxyphenylisocyanate (0.46 g, 2.3 mmol) and 1-pyrrolidinecarboxylic acid, 3-amino-, 1,1-dimethylethyl ester (0.46 g, 2.5 mmol) by method A. Product was purified by medium pressure chromatography (SiO₂, dichloromethane to 9/1 dichloromethane/methanol) to provide a solid. ¹H NMR (500 MHz, DMSO-d6) δ 8.52 (d, J=2.9 Hz, 1H), 7.50-7.42 (m, 2H), 7.25-7.18 (m, 2H), 6.50 (t, J=6.2 Hz, 1H), 4.12 (h, J=5.9 Hz, 1H), 3.44 (td, J=10.7, 5.4 Hz, 1H), 3.28 (dd, J=7.6, 3.8 Hz, 2H), 3.06 (dt, J=10.6, 3.8 Hz, 1H), 2.01 (tt, J=15.3, 7.6 Hz, 1H), 1.76 (tt, J=12.5, 6.2 Hz, 1H), 1.38 (d, J=3.9 Hz, 9H). HRMS ESI (+) calc'd for [M+H]=390.1636, observed=390.1658.

### Example 3: MSU-43307/ELE-3-60. tert-Butyl N-[(1-{[4-(trifluoromethoxy)phenyl]carbamoyl}pyrrolidin-3-yl)methyl]carbamate

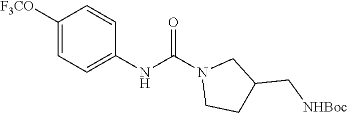 [SMILES: CC(C)(C)OC(=O)NCC1CCN(C(=O)Nc2ccc(OC(F)(F)F)cc2)C1]

The product was prepared in 72% yield (0.69 g) from 4-trifluoromethoxyphenylisocyanate (0.46 g, 2.3 mmol) and carbamic acid, N-(3-pyrrolidinylmethyl)-, 1,1-dimethylethyl ester (0.50 g, 2.5 mmol) via general method A. Product was purified by medium pressure chromatography (SiO₂, dichloromethane to 9/1 dichloromethane/methanol) to provide a solid. ¹H NMR (500 MHz, DMSO-d6) δ 8.29 (s, 1H), 7.63-7.57 (m, 2H), 7.20 (d, J=8.6 Hz, 2H), 6.99 (t, J=5.8 Hz, 1H), 3.46 (dd, J=10.4, 7.4 Hz, 2H), 3.32 (d, J=17.4 Hz, 1H), 3.04 (dd, J=10.5, 6.7 Hz, 1H), 2.94 (h, J=7.1 Hz, 2H), 2.30 (p, J=6.9 Hz, 1H), 1.91 (h, J=6.4 Hz, 1H), 1.59 (dt, J=12.4, 7.4 Hz, 1H), 1.36 (s, 9H). HRMS ESI (+) calc'd for [M+Na]=426.1640, observed=426.1640.

### Example 4: MSU-43308/ELE-3-65. tert-Butyl 3-[({[4-(trifluoromethoxy)phenyl]carbamoyl}amino)methyl]pyrrolidine-1-carboxylate

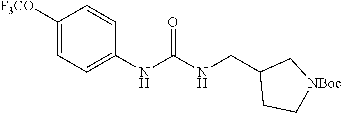 [SMILES: CC(C)(C)OC(=O)N1CCC(CNC(=O)Nc2ccc(OC(F)(F)F)cc2)C1]

The product was prepared in 91% yield from 4-trifluoromethoxyphenylisocyanate (0.46 g, 2.3 mmol) and 1-pyrrolidinecarboxylic acid, 3-(aminomethyl)-, 1,1-dimethylethyl ester (0.50 g, 2.5 mmol) via general method A. Product was purified by medium pressure chromatography (SiO₂, dichloromethane to 9/1 dichloromethane/methanol) to provide a solid. ¹H NMR (500 MHz, DMSO-d6) δ 8.61 (s, 1H), 7.50-7.42 (m, 2H), 7.24-7.18 (m, 2H), 6.33 (d, J=4.8 Hz, 1H), 3.31 (d, J=30.5 Hz, 1H), 3.23-2.88 (m, 5H), 2.28 (tt, J=14.4, 7.2 Hz, 1H), 1.86 (s, 1H), 1.53 (ddd, J=19.7, 11.8, 7.9 Hz, 1H), 1.37 (s, 9H). HRMS ESI (+) calc'd for [M+Na]=426.1617, found=426.1648.

### Example 5: MSU-43309/ELE-3-61. tert-Butyl N-[(1-{[4-(trifluoromethoxy)phenyl]carbamoyl}azetidin-3-yl)methyl]carbamate

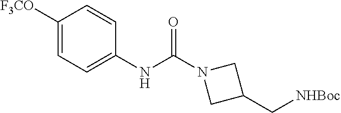 [SMILES: CC(C)(C)OC(=O)NCC1CN(C(=O)Nc2ccc(OC(F)(F)F)cc2)C1]

The product was prepared in 44% (0.42 g) yield from 4-trifluoromethoxyphenylisocyanate (0.46 g, 2.3 mmol) and carbamic acid,N-(3-azetidinylmethyl)-, 1,1-dimethylethyl ester (0.47 g, 2.5 mmol) via general method A. Product purified by medium pressure liquid chromatography (SiO₂, 9/1 hexanes/ethyl acetate to 100% ethyl acetate) to provide a solid. ¹H NMR (500 MHz, DMSO-d6) δ 8.55 (s, 1H), 7.61-7.54 (m, 2H), 7.25-7.18 (m, 2H), 7.06 (t, J=5.9 Hz, 1H), 3.92 (t, J=8.3 Hz, 2H), 3.62 (dd, J=8.4, 5.3 Hz, 2H), 3.12 (t, J=6.4 Hz, 2H), 2.60 (dt, J=14.3, 7.3 Hz, 1H), 1.36 (s, 9H). HRMS ESI (+) calc'd for [M+Na]=412.1460, observed=412.1480.

### Example 6: MSU-43310/ELE-3-63. tert-butyl 3-(3-(4-(trifluoromethoxy)phenyl)ureido)azetidine-1-carboxylate

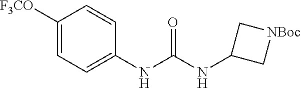 [SMILES: CC(C)(C)OC(=O)N1CC(NC(=O)Nc2ccc(OC(F)(F)F)cc2)C1]

The title compound was prepared in 72% yield from 4-trifluromethoxyphenyl isocyanate (0.46 g, 2.3 mmol) and 1-azetidinecarboxylic acid, 3-amino-, 1,7-dimethylethyl ester (047 g, 2.5 mmol) by general method A. The product was purified by medium pressure liquid chromatography (SiO₂, Hexanes to 5% Ethanol/Hexanes) to provide a solid. ¹H NMR (500 MHz, DMSO-d6) δ 8.78 (s, 1H), 7.51-7.44 (m, 2H), 7.25-7.19 (m, 2H), 6.89 (d, J=7.2 Hz, 1H), 4.36 (tdd, J=7.6, 5.5, 2.1 Hz, 1H), 4.05 (d, J=11.2 Hz, 2H), 3.69 (s, 2H), 1.36 (s, 9H). HRMS ESI (+) calc'd for [M+H]=376.1480, found=376.1499.

### Example 7: MSU-43311/ELE-3-64. tert-butyl 3-((3-(4-(trifluoromethoxy)phenyl)ureido)methyl)azetidine-1-carboxylate

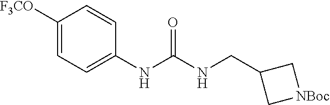 [SMILES: CC(C)(C)OC(=O)N1CC(CNC(=O)Nc2ccc(OC(F)(F)F)cc2)C1]

The product was prepared in 93% yield (0.82 g) from 4-trifluoromethoxyphenylisocyanate (0.46 g, 2.3 mmol) and 1-azetidinecarboxylic acid, 3-(aminomethyl)-, 1,1-dimethylethyl ester (0.47 g, 2.5 mmol) via general method A. The product was purified by medium pressure liquid chromatography (SiO₂, dichloromethane to 10% methanol/dichloromethane) to provide a solid. ¹H NMR (500 MHz, DMSO-d6) δ 8.69 (s, 1H), 7.50-7.42 (m, 2H), 7.23-7.18 (m, 2H), 6.39 (t, J=5.9 Hz, 1H), 3.83 (s, 2H), 3.25 (t, J=6.4 Hz, 2H), 2.67-2.56 (m, 1H), 1.34 (s, 9H). HRMS ESI (+) calc'd for [M+H]=390.1636, found=390.1660.

### Example 8: MSU-43472/MBG-VII-35. benzyl 3-(3-(4-(trifluoromethoxy)phenyl)ureido)azetidine-1-carboxylate

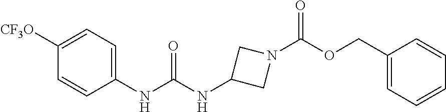

The title compound was prepared in 72% yield (0.73 g) from 4-trifluromethoxyphenyl isocyanate (0.51 g, 2.5 mmol) and 1-carbamoylbenzyloxy-3-aminoazetidine (0.44 g, 2.2 mmol) by general method A. Product was purified by medium pressure chromatography (SiO₂, dichloromethane to 9/1 dichloromethane/methanol) to provide a solid. ¹H NMR (500 MHzDMSO-d6) δ 8.80 (s, 1H), 7.52-7.43 (m, 2H), 7.40-7.27 (m, 5H), 7.25-7.16 (m, 2H), 6.88 (d, J=7.6 Hz, 1H), 5.03 (s, 2H), 4.44 (qt, J=7.7, 5.6 Hz, 1H), 4.15 (s, 2H). HRMS ESI (+) [M+Na] calc'd=410.1323, found=410.1348. 19F NMR (470 MHz, dmso) 6-57.15.

### Example 9: MSU-43793/MBG-VIII-52. tert-butyl ((1-((4-(trifluoromethyl)phenyl)carbamoyl)pyrrolidin-3-yl)methyl)carbamate

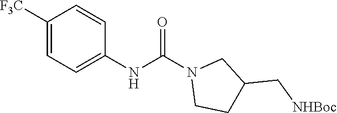 [SMILES: CC(C)(C)OC(=O)NCC1CCN(C(=O)Nc2ccc(C(F)(F)F)cc2)C1]

The title compound was prepared in 75% (1.2 g) yield from 4-trifluromethylphenylisocyanate (0.75 g, 4.0 mmol) and carbamic acid,N-(3-pyrrolidinylmethyl)-, 1,1-dimethylethyl ester (0.99 g, 4.9 mmol) using general method A. The crude product purified by silica gel chromatography (0 to 10% methanol in dichloromethane) and triturated from dichloromethane/hexanes. ¹H NMR (500 MHz, Chloroform-d) δ 7.52 (s, 4H), 6.38 (s, 1H), 4.72 (s, 1H), 3.61 (dt, J=11.4, 5.9 Hz, 2H), 3.46 (dt, J=9.7, 7.6 Hz, 1H), 3.19 (ddd, J=32.9, 12.0, 7.0 Hz, 3H), 2.49 (p, J=7.3 Hz, 1H), 2.16-2.08 (m, 1H), 1.76 (dq, J=12.5, 7.9 Hz, 1H), 1.45 (s, 9H). 19F NMR (470 MHz, Chloroform-d) 6-61.89. HRMS ESI (+) [M+H] calc'd for 388.1844, found=388.1844.

### Example 10: MSU-43834/MBG-8-115. tert-Butyl N-[(1-{[4-(pentafluoro-6-sulfanyl)phenyl]carbamoyl}pyrrolidin-3-yl)methyl]carbamate

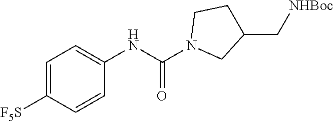

The isocyanate was prepared from 4-pentaflurosulfanylaniline and triphosgene using the method described in Zarei, M.; Vazquez Carrera, M.; Vazquez Cruz, S.; Leiva Martinez, R.; Pujol Bech, E. WO 2018/010856. 4-(pentafluoro)-A6-sulfonyl)aniline (0.350 g, 1.60 mmol) was dissolved in toluene (5.00 mL). Triphosgene (0.237 g, 0.800 mmol) was added, followed by triethylamine (0.161 g, 1.60 mmol). The reaction mixture was heated to 70° C. for 2 hours under an argon atmosphere. After cooling to room temperature hexanes (1.00 mL) was added to the reaction mixture, which was filtered through glass wool. The product was concentrated in vacuo to yield an oil which was used without further purification. The crude isocyanate was dissolved in dichloromethane (2.00 mL) and subsequently treated with carbamic acid,N-(3-pyrrolidinylmethyl)-,1,1-dimethylethyl ester using general method A (38%, 0.28 g). The mixture was concentrated and purified by silica gel chromatography (0 to 10% methanol in dichloromethane) and subsequently by reverse phase medium pressure liquid chromatography (25 mM aqueous ammonium formate/methanol (0 to 100%) using a C₁₈Aq RediSepRf Gold 50 g HP C₁₈Aq column). The product was then crystallized from dichloromethane/hexanes. ¹H NMR (500 MHz, DMSO-d6) δ 8.60 (s, 1H), 7.72 (s, 4H), 7.00 (t, J=5.8 Hz, 1H), 3.54-3.43 (m, 2H), 3.35 (s, 1H), 3.06 (dd, J=10.5, 6.8 Hz, 1H), 2.94 (p, J=6.3 Hz, 2H), 2.37-2.26 (m, 1H), 1.91 (dd, J=11.8, 5.7 Hz, 1H), 1.59 (dq, J=14.3, 7.5 Hz, 1H), 1.37 (s, 9H). HRMS ESI (+) calc'd for [M+Na]=468.1357, found=468.1367.

### Example 11: MSU-43386/ELE-3-73. 3-propanamido-N-[4-(trifluoromethoxy)phenyl]pyrrolidine-1-carboxamide

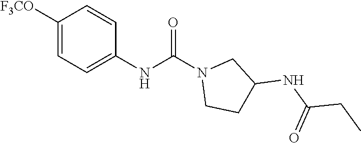 [SMILES: CCC(=O)NC1CCN(C(=O)Nc2ccc(OC(F)(F)F)cc2)C1]

Tert-butyl 3-({[4-(trifluoromethoxy)phenyl]carbamoyl}amino)pyrrolidine-1-carboxylate was deprotected to produce 3-amino-N-[4-(trifluoromethoxy)phenyl]pyrrolidine-1-carboxamide hydrochloride by general method B using a 4.0 M solution of hydrochloric acid in dioxide (0.53 mL, 2.1 mmol), then concentrated in vacuo. A portion (0.080 g, 0.25 mmol) was used to prepare the title compound in 55% yield (0.47 g) using propionic anhydride (0.13 mL, 0.98 mmol) and triethylamine (0.14 mL, 0.98 mmol) via general method B. Purified by medium pressure liquid chromatography (SiO₂, Dichloromethane to 10% Methanol/Dichloromethane) ¹H NMR (500 MHz, DMSO-d6) δ 8.33 (s, 1H), 8.04 (d, J=6.7 Hz, 1H), 7.63-7.57 (m, 2H), 7.23-7.18 (m, 2H), 4.23 (h, J=5.9 Hz, 1H), 3.58 (dd, J=10.6, 6.4 Hz, 1H), 3.51-3.37 (m, 2H), 3.19 (dd, J=10.6, 4.4 Hz, 1H), 2.11-1.99 (m, 3H), 1.77 (dq, J=12.6, 6.0 Hz, 1H), 0.97 (t, J=7.6 Hz, 3H). HRMS ESI (+) calc'd for [M+H]=346.1374, found=346.1383.

### Example 12: MSU-43365/ELE-3-76. 3-(propanamidomethyl)-N-[4-(trifluoromethoxy)phenyl]pyrrolidine-1-carboxamide

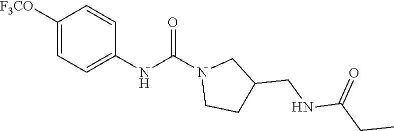 [SMILES: CCC(=O)NCC1CCN(C(=O)Nc2ccc(OC(F)(F)F)cc2)C1]

Tert-Butyl 3-[({[4-(trifluoromethoxy)phenyl]carbamoyl}amino)methyl]pyrrolidine-1-carboxylate was deprotected with a 4.0 N solution of hydrochloric acid (0.53 mL, 2.1 mmol) to produce crude 3-(aminomethyl)-N-[4-(trifluoromethoxy)phenyl]pyrrolidine-1-carboxamide hydrochloride by general method B then concentrated in vacuo. A portion (0.080 g, 0.24 mmol) was used to prepare the title compound in 84% yield (0.093 g) using propionic anhydride (0.13 mL, 0.98 mmol) and triethylamine (0.14 mL, 0.98 mmol) via general method B. Product was purified by medium pressure liquid chromatography (SiO₂, dichloromethane to 10% methanol/dichloromethane) to provide a solid. ¹H NMR (500 MHz, DMSO-d6) δ 8.30 (s, 1H), 7.88 (t, J=5.8 Hz, 1H), 7.63-7.55 (m, 2H), 7.20 (d, J=8.7 Hz, 2H), 3.46 (ddt, J=15.1, 9.7, 3.9 Hz, 3H), 3.13-2.99 (m, 3H), 2.37-2.29 (m, 1H), 2.07 (q, J=7.6 Hz, 2H), 1.93 (dt, J=12.6, 6.1 Hz, 1H), 1.59 (dq, J=15.3, 7.8 Hz, 1H), 0.98 (t, J=7.5 Hz, 3H). HRMS ESI (+) calc'd for [M+H]=360.1531, found=360.1551. m.p 166-167° C.

### Example 13: MSU-43385/ELE-3-74. 1-((1-propionylpyrrolidin-3-yl)methyl)-3-(4-(trifluoromethoxy)phenyl)urea

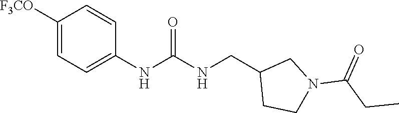 [SMILES: CCC(=O)N1CCC(CNC(=O)Nc2ccc(OC(F)(F)F)cc2)C1]

Tert-Butyl 3-[({[4-(trifluoromethoxy)phenyl]carbamoyl}amino)methyl]pyrrolidine-1-carboxylate was deprotected to produce crude N-[(pyrrolidin-3-yl)methyl]-N-[4-(trifluoromethoxy)phenyl]urea hydrochloride by general method B using a 4.0 M solution of hydrochloric acid in dioxide (0.53 mL, 2.1 mmol), and concentrated in vacuo. A portion (0.080 g, 0.24 mmol) was used to produce the title compound (94% yield, 0.079 g) when coupled with propionic anhydride (0.126 mL, 0.98 mmol) and triethylamine (0.14 mL, 0.98 mmol) via general method B. Product was purified by medium pressure liquid chromatography (SiO₂, Dichloromethane to 10% Methanol/Dichloromethane) to provide a solid. ¹H NMR (500 MHz, DMSO-d6) δ 8.63 (d, J=18.1 Hz, 1H), 7.50-7.42 (m, 2H), 7.23-7.17 (m, 2H), 6.35 (q, J=5.9 Hz, 1H), 3.53-3.35 (m, 2H), 3.25-2.95 (m, 4H), 2.40-2.24 (m, 1H), 2.19 (dtd, J=9.7, 7.6, 2.9 Hz, 2H), 1.99-1.82 (m, 1H), 1.58 (ddq, J=51.4, 12.4, 7.9 Hz, 1H), 0.95 (t, J=7.4 Hz, 3H). HRMS ESI (+) calc'd for [M+H]=360.1530, found=360.1544.

### Example 14: MSU-43366/ELE-3-80-B. 1-(1-propionylazetidin-3-yl)-3-(4-(trifluoromethoxy)phenyl)urea

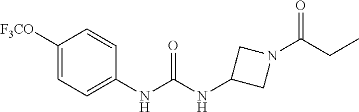 [SMILES: CCC(=O)N1CC(NC(=O)Nc2ccc(OC(F)(F)F)cc2)C1]

Using general method B, tert-butyl 3-({[4-(trifluoromethoxy)phenyl]carbamoyl}amino)azetidine-1-carboxylate was deprotected with a 4.0 N solution in dioxane of hydrochloric acid (0.53 mL, 2.1 mmol) to produce crude N-azetidin-3-yl-N-[4-(trifluoromethoxy)phenyl]urea hydrochloride. A portion (0.080, 0.26 mmol, 0.26 mmol) was used to produce the title compound 58% yield (0.049 g) when coupled to propionic anhydride (0.13 mL, 0.98 mmol) and triethylamine (0.14 mL, 0.98 mmol). The product was purified by medium pressure liquid chromatography (SiO₂, Dichloromethane to 5% Methanol/Dichloromethane) to provide a solid. ¹H NMR (500 MHz, DMSO-d6) δ 8.79 (s, 1H), 7.51-7.44 (m, 2H), 7.25-7.19 (m, 2H), 6.86 (d, J=7.1 Hz, 1H), 4.41 (qt, J=7.5, 5.4 Hz, 1H), 4.31 (td, J=8.2, 1.0 Hz, 1H), 4.05 (dd, J=9.6, 8.0 Hz, 1H), 3.91 (dd, J=8.6, 5.3 Hz, 1H), 3.67 (dd, J=9.7, 5.5 Hz, 1H), 2.03 (q, J=7.5 Hz, 2H), 0.94 (t, J=7.5 Hz, 3H). HRMS ESI (+) calc'd for [M+H]=332.1218, found=332.1242.

### Example 15: MSU-43392/ELE-3-83. 1-((1-propionylazetidin-3-yl)methyl)-3-(4-(trifluoromethoxy)phenyl)urea

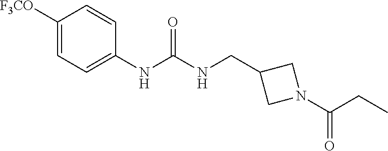 [SMILES: CCC(=O)N1CC(CNC(=O)Nc2ccc(OC(F)(F)F)cc2)C1]

Using general method B, t-butyl (trifluoromethoxy)phenyl]carbamoyl}pyrrolidin-3-yl)carbamate (0.18 g, 0.46 mmol) was deprotected with trifluoracetic acid (1.00 mL) to produce crude N-[(azetidin-3-yl)methyl]-N-[4-(trifluoromethoxy)phenyl]urea. This material was used, without further purification to produce the title compound (80% yield, 0.13 g) when mixed with propionic anhydride (0.18, 1.4 mmol) and triethylamine (0.51 mL, 3.6 mmol). The product was purified via medium pressure liquid chromatography (SiO₂, Dichloromethane to 5% Methanol/Dichloromethane) to provide a solid. ¹H NMR (500 MHz, DMSO-d6) δ 8.70 (s, 1H), 7.54-7.39 (m, 2H), 7.26-7.12 (m, 2H), 6.40 (t, J=5.9 Hz, 1H), 4.09 (t, J=8.3 Hz, 1H), 3.82 (t, J=8.9 Hz, 1H), 3.76 (dd, J=8.5, 5.3 Hz, 1H), 3.52 (dd, J=9.5, 5.4 Hz, 1H), 3.27 (t, J=6.4 Hz, 2H), 2.72-2.63 (m, 1H), 1.99 (q, J=7.5 Hz, 2H), 0.92 (t, J=7.5 Hz, 3H). HRMS ESI (+) calc'd for [M+H]=346.1374, found=346.1386.

### Example 16: MSU-43387/ELE-3-78. 1-(1-propionylpyrrolidin-3-yl)-3-(4-(trifluoromethoxy)phenyl)urea

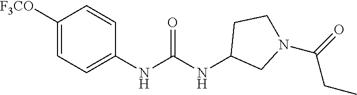 [SMILES: CCC(=O)N1CCC(NC(=O)Nc2ccc(OC(F)(F)F)cc2)C1]

Using general method B, tert-butyl 3-({[4-(trifluoromethoxy)phenyl]carbamoyl}amino)pyrrolidine-1-carboxylate (0.22 g, 0.57 mmol)) was deprotected with a 4.0 N solution in dioxane of hydrochloric acid (0.53 mL, 2.1 mmol) to produce, after being concentrated in vacuo. crude N-pyrrolidin-3-yl-N-[4-(trifluoromethoxy)phenyl]urea hydrochloride. A portion (0.080 g, 0.25 mmol) was used to produce the title compound (69% yield, 0.059 g) when mixed with propionic anhydride (0.130 mL, 0.980 mmol) and triethylamine (0.14 mL, 0.98 mmol). The product was purified via medium pressure liquid chromatography (SiO₂, dichloromethane to 5% methanol/dichloromethane) to provide a solid. ¹H NMR (500 MHz, DMSO-d6) δ 8.53 (d, J=15.6 Hz, 1H), 7.50-7.41 (m, 2H), 7.21 (dq, J=7.9, 1.2 Hz, 2H), 6.51 (dd, J=18.6, 6.8 Hz, 1H), 4.27-4.06 (m, 1H), 3.63 (dd, J=10.4, 6.1 Hz, 1H), 3.47 (dt, J=7.4, 5.4 Hz, 1H), 3.42-3.29 (m, 1H), 3.20 (ddd, J=24.1, 11.2, 4.5 Hz, 1H), 2.28-2.16 (m, 2H), 2.15-1.96 (m, 1H), 1.79 (ddq, J=58.1, 12.8, 6.0 Hz, 1H), 0.96 (q, J=7.4 Hz, 3H). HRMS ESI (+) calc'd for [M+H]=346.1374, found=346.1375.

### Example 17: MSU-43367/ELE-3-81. 1-(1-benzylpyrrolidin-3-yl)-3-(4-(trifluoromethoxy)phenyl)urea

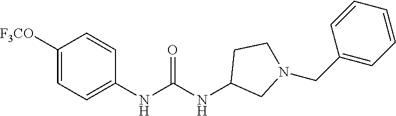 [SMILES: O=C(Nc1ccc(OC(F)(F)F)cc1)NC1CCN(Cc2ccccc2)C1]

A solution of tert-butyl 3-({[4-(trifluoromethoxy)phenyl]carbamoyl}amino)pyrrolidine-1-carboxylate was dissolved in methanol (3 mL) and treated with a 4.0 M solution of hydrochloric acid in dioxane (0.53 mL, 2.1 mmol). The mixture was stirred for 18 hours, then concentrated in vacuo to provide crude N-pyrrolidin-3-yl-N-[4-(trifluoromethoxy)phenyl]urea hydrochloride (0.185 g). A portion (0.090 g, 0.28 mmol) was, in a separate flask, dissolved in dichloromethane (2.0 mL) and treated with benzaldehyde (0.038, 0.36 mmol) and sodium triacetoxyborohydride (0.038 g, 0.61 mmol). The mixture was stirred overnight then quenched with water and extracted three times with ethyl acetate. The organic layers were combined, dried with sodium sulfate, filtered, and concentrated in vacuo. The crude product was purified by medium pressure chromatography (SiO₂, dichloromethane to 95/5 dichloromethane/methanol) to provide a solid (0.044 g, 40% yield). ¹H NMR (500 MHz, DMSO-d6) δ 8.55 (s, 1H), 7.47-7.40 (m, 2H), 7.30 (d, J=4.9 Hz, 4H), 7.25-7.15 (m, 3H), 6.37 (d, J=7.6 Hz, 1H), 4.10 (ddp, J=11.6, 7.9, 4.2 Hz, 1H), 3.62-3.50 (m, 2H), 2.66 (td, J=8.5, 4.8 Hz, 1H), 2.56 (dd, J=9.5, 6.5 Hz, 1H), 2.40-2.28 (m, 2H), 2.14 (dtd, J=13.3, 8.5, 4.9 Hz, 1H), 1.55-1.45 (m, 1H). HRMS ESI (+) calc'd for [M+H]=380.1582, found=380.1598.

### Example 18: MSU-43473/MBG-VII-54. 1-(1-pivaloylpyrrolidin-3-yl)-3-(4-(trifluoromethoxy)phenyl)urea

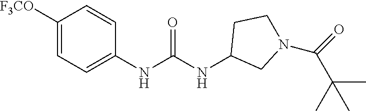 [SMILES: CC(C)(C)C(=O)N1CCC(NC(=O)Nc2ccc(OC(F)(F)F)cc2)C1]

Using general method B, tert-butyl 3-({[4-(trifluoromethoxy)phenyl]carbamoyl}amino)pyrrolidine-1-carboxylate (0.33 g, 0.85 mmol) and trifluoroacetic acid (2.0 mL) was deprotected to provide a salt once concentrated in vacuo. The crude intermediate was used in its entirety to produce the title compound in 23% yield (0.074 g) when mixed with trimethylacetyl chloride (0.24 g, 2.0 mmol) and triethylamine (0.51 g, 5.0 mmol) and purified by medium pressure liquid chromatography (SiO₂, 0 to 10% methanol in dichloromethane) and subsequently by reverse phase medium pressure liquid chromatography (25 mM aqueous ammonium formate/methanol (0 to 100%) using a C₁₈Aq RediSepRf Gold 50 g HP C₁₈Aq column) to provide a solid. ¹H NMR (500 MHz, Chloroform-d) δ 8.34 (s, 1H), 7.45-7.38 (m, 2H), 7.10 (d, J=8.6 Hz, 2H), 6.24 (s, 1H), 4.48 (s, 1H), 4.04 (s, 1H), 3.57 (s, 2H), 3.35 (s, 1H), 2.03 (s, 1H), 1.77 (s, 1H), 1.26 (s, 9H). 19F NMR (470 MHz, Chloroform-d) 6-58.25. HRMS ESI (+) [M+Na] calc'd=396.1511, found=396.1522.

### Example 19: MSU-43474/MBG-VII-69. 1-(1-isobutyrylpyrrolidin-3-yl)-3-(4-(trifluoromethoxy)phenyl)urea

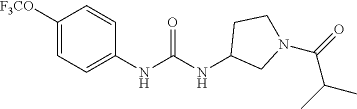 [SMILES: CC(C)C(=O)N1CCC(NC(=O)Nc2ccc(OC(F)(F)F)cc2)C1]

Using general method B, tert-butyl 3-({[4-(trifluoromethoxy)phenyl]carbamoyl}amino)pyrrolidine-1-carboxylate (0.600 g, 1.66 mmol) and trifluoroacetic acid (3.00 mL) was deprotected. The crude material was used in its entirety to produce the title compound in 37% yield (0.228 g) from isobutyryl chloride (0.319 g, 3.00 mmol) and triethylamine (0.404 g, 4.00 mmol) and purified by reverse phase medium pressure liquid chromatography (25 mM aqueous ammonium formate/methanol using a C₁₈Aq RediSepRf Gold 50 g HP C₁₈Aq column) and recrystallized from ethyl acetate/hexanes⁻¹H NMR (500 MHz, DMSO-d6) δ 8.55 (d, J=15.6 Hz, 1H), 7.51-7.40 (m, 2H), 7.21 (d, J=8.6 Hz, 2H), 6.50 (dd, J=18.9, 6.7 Hz, 1H), 4.17 (dq, J=47.0, 5.7 Hz, 1H), 3.68 (dd, J=10.4, 5.9 Hz, 1H), 3.55 (d, J=6.9 Hz, 1H), 3.47 (dd, J=12.1, 6.2 Hz, 1H), 3.40-3.34 (m, 1H), 3.16 (dd, J=12.0, 4.5 Hz, 1H), 2.63 (dh, J=27.2, 6.7 Hz, 1H), 2.16-1.96 (m, 1H), 1.80 (ddq, J=58.2, 12.3, 5.8 Hz, 1H), 1.05-0.94 (m, 6H). ¹⁹F NMR (470 MHz, DMSO-d6) δ −57.16. HRMS ESI (+) calc'd for [M+H]=360.1531, found=360.1540.

### Example 20: MSU-43773/MBG-8-89. 3-(pivalamidomethyl)-N-(4-(trifluoromethoxy)phenyl)pyrrolidine-1-carboxamide

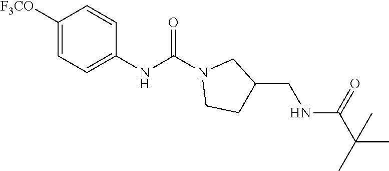 [SMILES: CC(C)(C)C(=O)NCC1CCN(C(=O)Nc2ccc(OC(F)(F)F)cc2)C1]

Using general method B, tert-butyl N-[(1-{[4-(trifluoromethoxy)phenyl]carbamoyl}pyrrolidin-3-yl)methyl]carbamate (0.400 g, 0.99 mmol) was deprotected with trifluoroacetic acid (3.00 mL) then concentrated in vacuo. The crude salt was used in its entirety to produce the title compound in 50% yield (0.190 g) using trimethylacetyl chloride (0.18 g, 1.5 mmol) and triethylamine (0.303 g, 3.00 mmol) and was purified by silica gel chromatography (0 to 10% methanol in dichloromethane). ¹H NMR (500 MHz, DMSO-d6) δ 9.74 (s, 1H), 8.31 (s, 1H), 7.60 (td, J=8.3, 6.9, 3.9 Hz, 2H), 7.20 (d, J=8.7 Hz, 1H), 3.44 (dd, J=10.6, 6.9 Hz, 1H), 3.19-2.90 (m, 6H), 2.42-2.34 (m, 1H), 1.95-1.83 (m, 1H), 1.59 (dq, J=15.4, 7.4 Hz, 1H), 1.18 (d, J=7.3 Hz, 4H), 1.08 (s, 6H). ¹⁹F NMR (470 MHz, DMSO-d6) δ −56.92 (d, J=7.8 Hz), −57.09. HRMS ESI (+) [M+H] calc'd for 410.1668, found=410.1667.

### Example 21: MSU-43774/MBG-8-92. 3-(isobutyramidomethyl)-N-(4-(trifluoromethoxy)phenyl)pyrrolidine-1-carboxamide

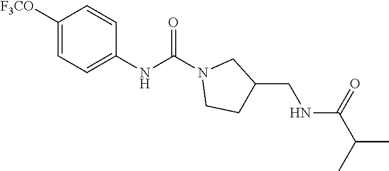 [SMILES: CC(C)C(=O)NCC1CCN(C(=O)Nc2ccc(OC(F)(F)F)cc2)C1]

Using general method B, tert-Butyl N-[(1-{[4-(trifluoromethoxy)phenyl]carbamoyl}pyrrolidin-3-yl)methyl]carbamate (0.400 g, 0.99 mmol) was deprotected with trifluoroacetic acid (3.00 mL). The title compound was prepared in 30% yield (0.110 g) using isobutyryl anhydride (0.474 g, 3.00 mmol) and triethylamine (0.404 g, 4.00 mmol) via general method B and purified by silica gel chromatography (0 to 10% methanol in dichloromethane) followed by recrystallization from dichloromethane/hexanes. ¹H NMR (500 MHz, DMSO-d6) δ 8.31 (s, 1H), 7.86 (t, J=5.8 Hz, 1H), 7.67-7.53 (m, 2H), 7.21 (d, J=8.7 Hz, 2H), 3.46 (ddd, J=12.6, 10.1, 6.0 Hz, 2H), 3.37-3.27 (m, 1H), 3.15-3.02 (m, 3H), 2.34 (dh, J=13.9, 7.0 Hz, 2H), 1.93 (dq, J=12.3, 6.3 Hz, 1H), 1.60 (dq, J=12.1, 7.7 Hz, 1H), 0.99 (d, J=6.8 Hz, 6H). ¹⁹F NMR (470 MHz, DMSO-d6) δ −56.93. HRMS ESI (+) [M+H] calc'd for 374.1687, found=374.1699.

### Example 22: MSU-43705/ELE-3-91. 3-{[(Pyridin-2-yl)amino]methyl}-N-[4-(trifluoromethoxy)phenyl]pyrrolidine-1-carboxamide

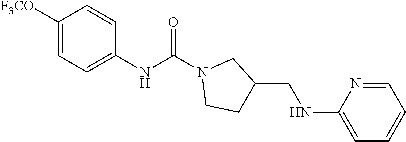 [SMILES: O=C(Nc1ccc(OC(F)(F)F)cc1)N1CCC(CNc2ccccn2)C1]

tert-Butyl 3-[({[4-(trifluoromethoxy)phenyl]carbamoyl}amino)methyl]pyrrolidine-1-carboxylate was deprotected with a 4.0 N solution of hydrochloric acid (0.53 mL, 2.1 mmol) in dioxane to produce crude 3-(aminomethyl)-N-[4-(trifluoromethoxy)phenyl]pyrrolidine-1-carboxamide hydrochloride by general method B. The title compound was prepared in yield by mixing crude amine hydrochloride (0.19 g, 0.55 mmol), 2-fluoropyridine (0.21 g, 2.2 mmol) and diisopropylethylamine (0.57 mL, 3.3 mmol) in dimethylsulfoxide (6.0 mL) and heating the mixture with an Anton-Parr Monowave 400 to 120° C. for 12 hrs. The mixture was cooled, diluted with water, and extracted with ethyl acetate. The organic layer was then washed two times with water, dried with sodium sulfate, filtered, and concentrated in vacuo. The crude was then purified by medium pressure chromatography (SiO₂, dichloromethane to 90/10 dichloromethane/methanol) to provide the product (86% yield, 0.180 g). ¹H NMR (500 MHz, Chloroform-d) δ 8.11-8.04 (m, 1H), 7.52-7.40 (m, 2H), 7.39-7.31 (m, 2H), 7.15-7.04 (m, 2H), 6.56 (ddd, J=7.1, 5.2, 1.0 Hz, 1H), 6.38 (dt, J=8.6, 1.0 Hz, 1H), 5.56 (s, 1H), 3.64 (dd, J=10.3, 7.3 Hz, 1H), 3.56-3.49 (m, 1H), 3.46-3.34 (m, 2H), 3.28 (dd, J=10.3, 6.7 Hz, 1H), 3.22 (ddd, J=13.7, 7.9, 5.8 Hz, 1H), 2.57 (dq, J=14.0, 7.0 Hz, 1H), 2.19-2.08 (m, 1H), 1.78 (dq, J=12.4, 7.9 Hz, 1H). HRMS ESI (+) [M+H] calc'd for 381.1534, found=381.1562.

### Example 23: MSU-43775/MBG-8-65. 3-(propanamidomethyl)-N-[4-(trifluoromethyl)phenyl]pyrrolidine-1-carboxamide

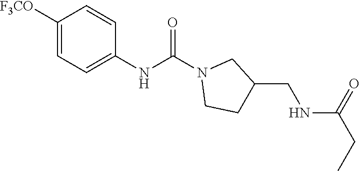 [SMILES: CCC(=O)NCC1CCN(C(=O)Nc2ccc(OC(F)(F)F)cc2)C1]

Using general method B, tert-butyl N-[(1-{[4-(trifluoromethyl)phenyl]carbamoyl}pyrrolidin-3-yl)methyl]carbamate ((0.084 g, 0.260 mmol) was deprotected with trifluoroacetic acid (3.00 mL) then concentrated in vacuo. The title compound was prepared in 34% yield using the crude salt, propionyl anhydride (0.169 g, 1.30 mmol) and triethylamine (0.263 g, 2.60 mmol). The final product was purified by reverse phase medium pressure liquid chromatography (25 mM aqueous ammonium formate/methanol (0 to 100%) using a C₁₈Aq RediSepRf Gold 50 g HP C₁₈Aq column). ¹H NMR (500 MHz, Chloroform-d) δ 7.53 (s, 4H), 6.42 (s, 1H), 5.84 (s, 1H), 3.61 (qd, J=8.2, 7.1, 4.5 Hz, 2H), 3.49-3.37 (m, 2H), 3.29-3.20 (m, 2H), 2.51 (hept, J=7.1 Hz, 1H), 2.14-2.05 (m, 1H), 1.75 (dq, J=12.4, 7.9 Hz, 1H), 1.21 (s, 9H). HRMS ESI (+) [M+H] calc'd for 344.1582, found=344.1585.

### Example 24: MSU-43776/MBG-8-93. 3-(isobutyramidomethyl)-N-(4-(trifluoromethyl)phenyl)pyrrolidine-1-carboxamide

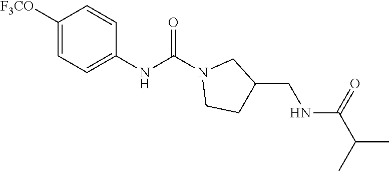 [SMILES: CC(C)C(=O)NCC1CCN(C(=O)Nc2ccc(OC(F)(F)F)cc2)C1]

Using general method B, tert-Butyl N-[(1-{[4-(trifluoromethyl)phenyl]carbamoyl}pyrrolidin-3-yl)methyl]carbamate (0.250 g, 0.77 mmol) was deprotected with trifluoroacetic acid (2.00 mL) and concentrated in vacuo. The crude material was used in its entirety to produce the title compound in 27% yield (0.075 g) using the crude salt and triethylamine (0.404 g, 4.00 mmol). The product was purified by silica gel chromatography (0 to 10% methanol in dichloromethane) and subsequently by reverse phase medium pressure liquid chromatography (25 mM aqueous ammonium formate/methanol (0 to 100%) using a C₁₈Aq RediSepRf Gold 50 g HP C₁₈Aq column). The final product was recrystallized from dichloromethane/hexanes. ¹H NMR (500 MHz, DMSO-d6) δ 8.50 (s, 1H), 7.86 (t, J=5.6 Hz, 1H), 7.72 (d, J=8.5 Hz, 2H), 7.55 (d, J=8.5 Hz, 2H), 3.53-3.42 (m, 2H), 3.36 (d, J=7.5 Hz, 1H), 3.07 (tdd, J=13.2, 11.3, 6.7 Hz, 3H), 2.33 (tt, J=10.3, 6.8 Hz, 2H), 1.92 (dq, J=12.6, 6.6 Hz, 1H), 1.66-1.52 (m, 1H), 0.98 (d, J=6.9 Hz, 6H). ¹⁹F NMR (470 MHz, DMSO-d6) δ 59.97. HRMS ESI (+) [M+H] calc'd for 358.1738, found=358.1753.

### Example 25: MSU-43777/MBG-8-66. 3-(pivalamidomethyl)-N-(4-(trifluoromethyl)phenyl)pyrrolidine-1-carboxamide

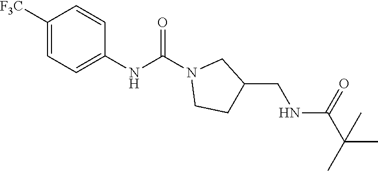 [SMILES: CC(C)(C)C(=O)NCC1CCN(C(=O)Nc2ccc(C(F)(F)F)cc2)C1]

Using general method B, tert-Butyl N-[(1-{[4-(trifluoromethyl)phenyl]carbamoyl}pyrrolidin-3-yl)methyl]carbamate (0.10 g, 0.31 mmol) was deprotected with trifluoroacetic acid (2.00 mL) and concentrated in vacuo. The crude material was used in its entirety to produce the title compound in 43% yield (0.049 g) using trifluoromethylacetyl chloride (0.19 g, 1.6 mmol) and triethylamine (0.313 g, 3.10 mmol). The final product was purified by medium pressure liquid chromatography (SiO₂, 0 to 10% methanol in dichloromethane). ¹H NMR (500 MHz, DMSO-d6) δ 8.50 (s, 1H), 7.89 (t, J=5.7 Hz, 1H), 7.72 (d, J=8.6 Hz, 2H), 7.55 (d, J=8.6 Hz, 2H), 3.55-3.44 (m, 2H), 3.36 (d, J=7.6 Hz, 1H), 3.13-3.01 (m, 3H), 2.32 (p, J=7.2 Hz, 1H), 2.07 (q, J=7.6 Hz, 2H), 1.93 (dq, J=12.5, 6.6 Hz, 1H), 1.67-1.56 (m, 1H), 0.98 (t, J=7.6 Hz, 3H). ¹⁹F NMR (470 MHz, DMSO-d6) δ −59.81, −59.94, −59.97, −60.01, −60.39, −61.24, −61.30 (d, J=3.2 Hz). HRMS ESI (+) [M+H] calc'd for 372.1895, found=372.1904.

### Example 26: MSU-43893/MB G-8-119. 3-((3-methylbutanamido)methyl)-N-(4-(trifluoromethyl)phenyl)pyrrolidine-1-carboxamide

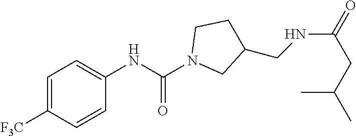 [SMILES: CC(C)CC(=O)NCC1CCN(C(=O)Nc2ccc(C(F)(F)F)cc2)C1]

Using general method B, tert-Butyl N-[(1-{[4-(trifluoromethyl)phenyl]carbamoyl}pyrrolidin-3-yl)methyl]carbamate (0.125 g, 0.380 mmol) was deprotected with trifluoroacetic acid (2.00 mL) and concentrated in vacuo. The crude material was used in its entirety to produce the title compound in 16% yield (0.022 g) using isovaleric anhydride (0.372 g, 2.00 mmol) and triethylamine (0.707 g, 7.00 mmol). The product was purified medium pressure liquid chromatography (SiO₂, 0 to 10% methanol in dichloromethane) and subsequently by reverse phase medium pressure liquid chromatography (25 mM aqueous ammonium formate/methanol (0 to 100%) using a C₁₈Aq RediSepRf Gold 50 g HP C₁₈Aq column) to provide a solid. ¹H NMR (500 MHz, DMSO-d6) δ 8.50 (s, 1H), 7.92 (t, J=5.8 Hz, 1H), 7.72 (d, J=8.5 Hz, 2H), 7.55 (d, J=8.5 Hz, 2H), 3.48 (td, J=9.8, 5.9 Hz, 2H), 3.37 (d, J=7.7 Hz, 1H), 3.14-2.99 (m, 3H), 2.34 (dt, J=21.3, 7.1 Hz, 1H), 2.01-1.88 (m, 4H), 1.61 (dq, J=15.2, 7.8 Hz, 1H), 0.94-0.76 (m, 6H). ¹⁹F NMR (470 MHz, DMSO-d6) δ −59.97. HRMS ESI (+) calc'd for [M+H]=372.1895, found=372.1919.

### Example 27: MSU-43894/MB G-8-120. 3-[(3-methylbutanamido)methyl]-N-[4-(trifluoromethoxy)phenyl]pyrrolidine-1-carboxamide

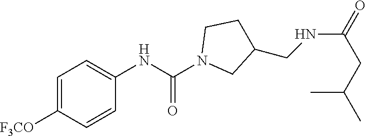 [SMILES: CC(C)CC(=O)NCC1CCN(C(=O)Nc2ccc(OC(F)(F)F)cc2)C1]

Using general method B, tert-Butyl N-[(1-{[4-(trifluoromethoxy)phenyl]carbamoyl}pyrrolidin-3-yl)methyl]carbamate (0.200 g, 0.490 mmol) was deprotected with trifluoroacetic acid (2.00 mL) and concentrated in vacuo. The crude material was used in its entirety to produce the title compound in 64% (0.33 g) yield using isovaleric anhydride (0.37 g, 2.0 mmol) and triethylamine (0.303 g, 3.00 mmol). The final product was purified by medium pressure liquid chromatography (SiO₂, 0 to 10% methanol in dichloromethane) and subsequently by reverse phase medium pressure liquid chromatography (25 mM aqueous ammonium formate/methanol using a C₁₈Aq RediSepRf Gold 50 g HP C₁₈Aq column) to provide a solid. ¹H NMR (500 MHz, Methanol-d4) δ 7.55-7.45 (m, 2H), 7.21-7.12 (m, 2H), 3.59 (ddd, J=11.6, 9.3, 5.9 Hz, 2H), 3.45 (dt, J=10.2, 7.6 Hz, 1H), 3.29-3.23 (m, 2H), 3.17 (ddd, J=10.3, 7.0, 3.2 Hz, 2H), 2.54-2.43 (m, 1H), 2.16-2.03 (m, 3H), 1.81-1.69 (m, 1H), 0.95 (d, J=6.0 Hz, 6H). 19F NMR (470 MHz, Methanol-d4) δ −59.84. HRMS ESI (+) calc'd for [M+H]=388.1844, found=388.1866.

### Example 28: MSU-43895/MBG-8-121. 3-((3-methylbutanamido)methyl)-N-(4-(pentafluoro-λ6-sulfaneyl)phenyl)pyrrolidine-1-carboxamide

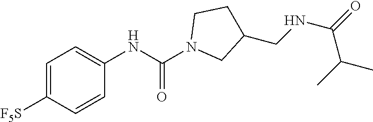

Using general method B, tert-Butyl N-[(1-{[4-(pentafluorosulfanyl)phenyl]carbamoyl}pyrrolidin-3-yl)methyl]carbamate was deprotected with trifluoroacetic acid (1.00 mL). The crude material was used in its entirety to produce the title compound in 46% (0.054 g) using tert-butyl-N-[(1-{[4-(pentafluorosulfanyl)phenyl]carbamoyl}pyrrolidin-3-yl)methyl]carbamate (0.125 g, 0.280 mmol), isobutyric anhydride (0.186 g, 1.00 mmol), and triethylamine (0.202 g, 2.00 mmol). The final product was purified by silica gel chromatography (0 to 10% methanol in dichloromethane) and subsequently by reverse phase medium pressure liquid chromatography (25 mM aqueous ammonium formate/methanol using a C₁₈Aq RediSepRf Gold 50 g HP C₁₈Aq column). ¹H NMR (500 MHz, DMSO-d6) δ 8.60 (s, 1H), 7.86 (t, J=5.8 Hz, 1H), 7.72 (d, J=1.8 Hz, 4H), 3.54-3.43 (m, 2H), 3.06 (dtt, J=15.8, 13.2, 6.3 Hz, 3H), 2.39-2.29 (m, 2H), 1.92 (dq, J=13.3, 6.7 Hz, 1H), 1.60 (dq, J=15.3, 7.7 Hz, 1H), 0.98 (d, J=6.8 Hz, 6H). HRMS ESI (+) calc'd for [M+H]=416.1427, found=416.1438.

### Example 29: MSU43869/KSL-0013. Tert-butyl 3,3-difluoro-4-(3-(4 (trifluoromethoxy)phenyl)ureido)piperidine-1-carboxylate

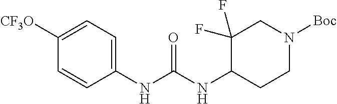

The title compound was prepared in 58% yield (50.45 mg) using 4-(Trifluoromethoxy)phenyl isocyanate (0.0424 g, 0.209 mmol) and tert-butyl 4-amino-3,3-difluoropiperidine-1-carboxylate (0.0540 g, 0.229 mmol) via general method C. The final product was purified by silica gel chromatography (EtOAc:Hex/1:1). ¹H NMR (500 MHz, DMSO-d6) δ 8.70 (s, 1H), 7.52-7.43 (m, 2H), 7.29-7.21 (m, 2H), 6.57 (d, J=9.3 Hz, 1H), 4.28 (s, 1H), 3.91 (s, 1H), 3.15-2.93 (m, 1H), 1.86 (d, J=12.9 Hz, 1H), 1.41 (s, 10H), 1.30-1.08 (m, 2H).

### Example 30: MSU43870/KSL-0014. Tert-butyl (3S,4R)-3-fluoro-4-(3-(4 (trifluoromethoxy)phenyl)ureido)piperidine-1-carboxylate

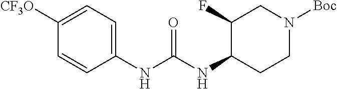

The title compound was prepared in 53% yield (92.79 mg) using 4-(Trifluoromethoxy)phenyl isocyanate (0.0840 g, 0.414 mmol) and cis-4-amino-1-boc-3-fluoropiperidine (0.0992 g, 0.455 mmol) via general method C. The final product was purified by silica gel chromatography (EtOAc:Hex/1:1). ¹H NMR (500 MHz, DMSO-d6) δ 8.69 (s, 1H), 7.49-7.43 (m, 2H), 7.27-7.18 (m, 2H), 6.41 (d, J=8.4 Hz, 1H), 4.82-4.62 (m, 1H), 4.17 (s, 1H), 4.04-3.79 (m, 2H), 3.25-2.71 (m, 2H), 1.65 (dd, J=12.9, 4.4 Hz, 1H), 1.51 (qd, J=12.6, 4.5 Hz, 1H), 1.39 (s, 9H).

### Example 31: MSU-43871/KSL-0015. Tert-butyl (3R,4S)-3-fluoro-4-(3-(4-(trifluoromethoxy)phenyl)ureido)piperidine-1-carboxylate

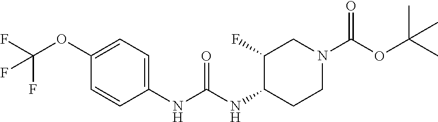 [SMILES: CC(C)(C)OC(=O)N1CC[C@H](NC(=O)Nc2ccc(OC(F)(F)F)cc2)[C@H](F)C1]

The title compound was prepared in 72% yield (119.18 mg) using 4-(Trifluoromethoxy)phenyl isocyanate (0.0800 g, 0.394 mmol) and tert-butyl (3R,4S)-4-amino-3-fluoropiperidine-1-carboxylate (0.0945 g, 0.433 mmol) via general method C. The final product was purified by silica gel chromatography (EtOAc:Hex/1:1). ¹H NMR (500 MHz, DMSO-d6) δ 8.69 (s, 1H), 7.52-7.42 (m, 2H), 7.28-7.19 (m, 2H), 6.41 (d, J=8.4 Hz, 1H), 4.81-4.61 (m, 1H), 4.17 (s, 1H), 4.04-3.79 (m, 2H), 3.25-2.74 (m, 2H), 1.69-1.61 (m, 1H), 1.51 (qd, J=12.5, 4.5 Hz, 1H), 1.39 (s, 9H).

### Example 32: MSU-43872/KSL-0016. Tert-butyl (3S,4S)-3-fluoro-4-(3-(4-(trifluoromethoxy)phenyl)ureido)piperidine-1-carboxylate

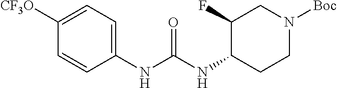

The title compound was prepared in 45% yield (77.75 mg) using 4-(Trifluoromethoxy)phenyl isocyanate (0.0840 g, 0.414 mmol) and (3S,4S)-tert-Butyl 4-amino-3-fluoropiperidine-1-carboxylate (0.0992 g, 0.455 mmol) via general method C. The final product was purified by silica gel chromatography (EtOAc:Hex/1:1). ¹H NMR (500 MHz, DMSO-d6) δ 8.69 (s, 1H), 7.52-7.44 (m, 2H), 7.23 (d, J=8.7 Hz, 2H), 6.47 (d, J=8.0 Hz, 1H), 4.51-4.33 (m, 1H), 3.94-3.75 (m, 2H), 3.57 (s, 1H), 3.13 (ddd, J=13.3, 9.6, 3.4 Hz, 2H), 1.88 (dtd, J 9.7, 6.7, 2.8 Hz, 1H), 1.40 (s, 10H).

### Example 33: MSU-43873/KSL-0017. Tert-butyl (2R,4S)-2-methyl-4-(3-(4-(trifluoromethoxy)phenyl)ureido)piperidine-1-carboxylate

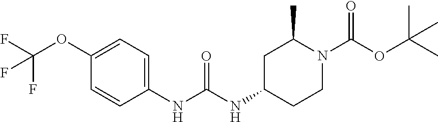 [SMILES: C[C@@H]1C[C@@H](NC(=O)Nc2ccc(OC(F)(F)F)cc2)CCN1C(=O)OC(C)(C)C]

The title compound was prepared in 27% yield (118.43 mg) using 4-(Trifluoromethoxy)phenyl isocyanate (0.215 g, 1.058 mmol) and (2R,4S)-rel-tert-Butyl 4-amino-2-methylpiperidine-1-carboxylate (0.250 g, 1.167 mmol) via general method C. The final product was purified by silica gel chromatography (EtOAc:Hex/1:1). ¹H NMR (500 MHz, DMSO-d6) δ 8.56 (s, 1H), 7.50-7.41 (m, 2H), 7.21 (d, J=8.6 Hz, 2H), 6.10 (d, J=7.8 Hz, 1H), 4.33 (s, 1H), 3.89-3.73 (m, 2H), 1.84 (d, J=12.6 Hz, 1H), 1.71 (d, J=12.9 Hz, 1H), 1.39 (s, 10H), 1.27-1.05 (m, 5H).

### Example 34: MSU-438 74/KSL-0018. Tert-butyl (2R,4R)-2-methyl-4-(3-(4-(trifluoromethoxy)phenyl)ureido)piperidine-1-carboxylate

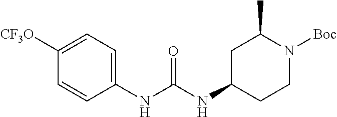

The title compound was prepared in 67% yield (136.88 mg) using 4-(Trifluoromethoxy)phenyl isocyanate (0.0856 g, 0.421 mmol) and tert-Butyl (2R,4R)-4-amino-2-methylpiperidine-1-carboxylate (0.0993 g, 0.463 mmol) via general method C. The final product was purified by silica gel chromatography (EtOAc:Hex/1:1). ¹H NMR (500 MHz, DMSO-d6) δ 8.62 (s, 1H), 7.51-7.44 (m, 2H), 7.27-7.17 (m, 2H), 6.27 (d, J=6.7 Hz, 1H), 4.03 (td, J=6.8, 4.7 Hz, 1H), 3.77 (h, J=5.1 Hz, 1H), 3.68 (ddd, J=13.8, 5.4, 3.3 Hz, 1H), 3.06 (ddd, J=13.7, 11.8, 3.7 Hz, 1H), 1.87-1.68 (m, 2H), 1.66-1.51 (m, 2H), 1.40 (s, 9H), 1.19 (d, J 6.9 Hz, 3H).

### Example 35: MSU-43876/KSL-0020. Tert-butyl (1R,3s,5S)-3-(3-(4-(trifluoromethoxy)phenyl)ureido)-8-azabicyclo[3.2.1]octane-8-carboxylate

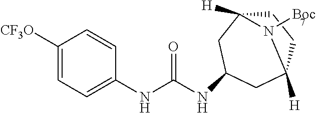

The title compound was prepared in 57% yield (121.02 mg) using 4-(Trifluoromethoxy)phenyl isocyanate (0.100 g, 0.492 mmol) and tert-butyl (1R,3s,5S)-3-amino-8-azabicyclo[3.2.1]octane-8-carboxylate (0.123 g, 0.543 mmol) via general method C. The final product was purified by silica gel chromatography (EtOAc:Hex/1:1). ¹H NMR (500 MHz, DMSO-d6) δ 8.43 (s, 1H), 7.48-7.43 (m, 2H), 7.23-7.18 (m, 2H), 6.17 (d, J=8.1 Hz, 1H), 4.06 (s, 2H), 4.00 (ddd, J=11.3, 7.9, 5.7 Hz, 1H), 1.96-1.72 (m, 4H), 1.72-1.61 (m, 2H), 1.42 (s, 11H).

### Example 36: MSU-43877/KSL-0021. Tert-butyl (1R,3r,5S)-3-(3-(4-(trifluoromethoxy)phenyl)ureido)-8-azabicyclo[3.2.1]octane-8-carboxylate

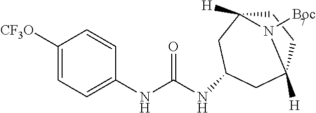

The title compound was prepared in 59% (111.74 mg) yield using 4-(Trifluoromethoxy)phenyl isocyanate (0.0900 g, 0.443 mmol) and tert-butyl (1R,3r,5S)-3-amino-8-azabicyclo[3.2.1]octane-8-carboxylate (0.110 g, 0.486 mmol) via general method C. The final product was purified by silica gel chromatography (EtOAc:Hex/1:1). ¹H NMR (500 MHz, DMSO-d6) δ 8.70 (s, 1H), 7.50-7.44 (m, 2H), 7.25-7.18 (m, 2H), 6.41 (d, J=6.5 Hz, 1H), 4.08-3.99 (m, 2H), 3.87 (q, J=6.6 Hz, 1H), 2.00 (d, J=15.8 Hz, 2H), 1.94 (s, 4H), 1.61 (s, 2H), 1.40 (s, 9H).

### Example 37: MSU-43888/KSL-0032. Tert-butyl (2-((4-(trifluoromethoxy)phenyl)carbamoyl)-2-azaspiro[3.3]heptan-6-yl)carbamate

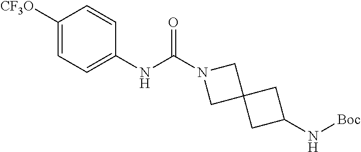

The title compound was prepared in 66% (120.56 mg) yield using 4-(Trifluoromethoxy)phenyl isocyanate (0.0900 g, 0.443 mmol) and tert-butyl (2-azaspiro[3.3]heptan-6-yl)carbamate (0.103 g, 0.485 mmol) via general method C. The final product was purified by silica gel chromatography (EtOAc:Hex/1:1). ¹H NMR (500 MHz, DMSO-d6) δ 8.57 (s, 1H), 7.60-7.55 (m, 2H), 7.25-7.20 (m, 2H), 7.13 (d, J=7.9 Hz, 1H), 3.96 (s, 2H), 3.84 (s, 2H), 3.83-3.76 (m, 1H), 2.40 (ddd, J=10.0, 7.7, 2.9 Hz, 2H), 2.05 (td, J=8.9, 2.9 Hz, 2H), 1.36 (s, 9H).

### Example 38: MSU-43885/KSL-0029. Tert-butyl 6-(3-(4-(trifluoromethoxy)phenyl)ureido)-2-azaspiro[3.3]heptane-2-carboxylate

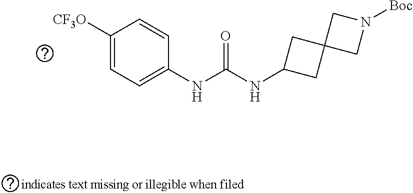

The title compound was prepared in 64% yield (121.10 mg) using 4-(Trifluoromethoxy)phenyl isocyanate (0.0930 g, 0.458 mmol) and tert-butyl 6-amino-2-azaspiro[3.3]heptane-2-carboxylate (0.107 g, 0.504 mmol) via general method C. The final product was purified by silica gel chromatography (EtOAc:Hex/1:1). ¹H NMR (500 MHz, DMSO-d6) δ 8.55 (s, 1H), 7.48-7.43 (m, 2H), 7.23-7.18 (m, 2H), 6.44 (d, J=7.7 Hz, 1H), 3.98 (h, J=8.0 Hz, 1H), 3.87 (s, 2H), 3.76 (s, 2H), 2.45 (ddt, J=12.0, 7.7, 2.0 Hz, 2H), 2.03 (ddt, J=11.0, 8.4, 2.1 Hz, 2H), 1.36 (s, 9H).

### Example 39: MSU-43882/KSL-0026. Tert-butyl (1R,5S,6s)-6-(3-(4-(trifluoromethoxy)phenyl)ureido)-3-azabicyclo[3.1.0]hexane-3-carboxylate

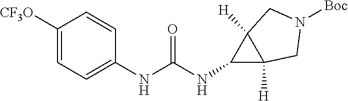

The title compound was prepared in 68% yield (145.53 mg) using 4-(Trifluoromethoxy)phenyl isocyanate (0.108 g, 0.532 mmol) and tert-butyl (1R,5S,6s)-6-amino-3-azabicyclo[3.1.0]hexane-3-carboxylate (0.116 g, 0.585 mmol) via general method C. The final product was purified by silica gel chromatography (EtOAc:Hex/1:1). ¹H NMR (500 MHz, DMSO-d6) δ 8.59 (s, 1H), 7.53-7.46 (m, 2H), 7.25-7.19 (m, 2H), 6.51 (s, 1H), 3.53-3.46 (m, 2H), 3.28 (s, 2H), 2.24-2.20 (m, 1H), 1.65 (q, J=2.1 Hz, 2H), 1.38 (s, 9H).

### Example 40: MSU-43886/KSL-0030. Tert-butyl (1R,5S,6r)-6-(3-(4-(trifluoromethoxy)phenyl)ureido)-3-azabicyclo[3.1.0]hexane-3-carboxylate

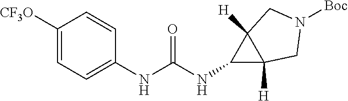

The title compound was prepared in 61% (120.86 mg) yield using 4-(Trifluoromethoxy)phenyl isocyanate (0.100 g, 0.492 mmol) and tert-butyl (1R,5S,6r)-6-amino-3-azabicyclo[3.1.0]hexane-3-carboxylate (0.105 g, 0.530 mmol) via general method C. The final product was purified by silica gel chromatography (EtOAc:Hex/1:1). ¹H NMR (500 MHz, DMSO-d6) δ 8.68 (s, 1H), 7.50-7.45 (m, 2H), 7.25-7.20 (m, 2H), 6.15 (s, 1H), 3.45 (ddd, J=11.2, 6.6, 4.8 Hz, 2H), 3.32-3.22 (m, 2H), 2.68 (td, J=7.0, 2.3 Hz, 1H), 1.80 (h, J=7.5 Hz, 2H), 1.32 (s, 9H).

### Example 41: MSU-43887/KSL-0031. Tert-butyl (3-((4-(trifluoromethoxy)phenyl)carbamoyl)-3-azabicyclo[3.1.0]hexan-6-yl)carbamate

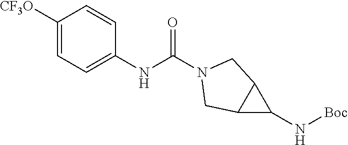

The title compound was prepared in 75% (148.32 mg) yield using 4-(Trifluoromethoxy)phenyl isocyanate (0.100 g, 0.492 mmol) and tert-butyl (3-azabicyclo[3.1.0]hexan-6-yl)carbamate (0.107 g, 0.540 mmol) via general method C. The final product was purified by silica gel chromatography (EtOAc:Hex/1:1). ¹H NMR (500 MHz, DMSO-d6) δ 8.31 (s, 1H), 7.63-7.54 (m, 2H), 7.25-7.18 (m, 2H), 3.64 (d, J=10.5 Hz, 1H), 3.52 (s, 1H), 3.40 (d, J=10.3 Hz, 1H), 1.80 (s, 1H), 1.67 (d, J=3.4 Hz, 1H), 1.47-1.28 (m, 9H).

### Example 42: MSU-43996/KSL-0033. Tert-butyl (1R,4R,5S)-5-(3-(4-(trifluoromethoxy)phenyl)ureido)-2-azabicyclo[2.1.1]hexane-2-carboxylate

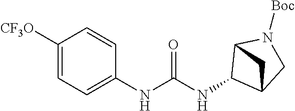

The title compound was prepared in 84% yield (166.62 mg) using 4-(Trifluoromethoxy)phenyl isocyanate (0.100 g, 0.492 mmol) and tert-butyl (1R,4R,5S)-5-amino-2-azabicyclo[2.1.1]hexane-2-carboxylate (0.100 g, 0.504 mmol) via general method C. The final product was purified by silica gel chromatography (EtOAc:Hex/1:1). ¹H NMR (500 MHz, DMSO-d6) δ 8.93 (s, 1H), 7.50-7.40 (m, 2H), 7.21 (d, J=8.6 Hz, 2H), 6.40 (d, J=7.8 Hz, 1H), 3.64 (dt, J=7.9, 2.3 Hz, 1H), 3.50 (dt, J=5.9, 1.8 Hz, 1H), 2.78 (d, J=8.6 Hz, 1H), 2.64 (td, J=6.7, 3.3 Hz, 2H), 1.43-1.32 (m, 1H), 0.93 (d, J=7.6 Hz, 1H).

### Example 43: MSU-43997/KSL-0034. Tert-butyl (1R,4R,7S)-7-(3-(4-(trifluoromethoxy)phenyl)ureido)-2-azabicyclo[2.2.1]heptane-2-carboxylate

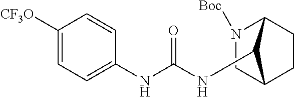

The title compound was prepared in 82% yield (167.12 mg) using 4-(Trifluoromethoxy)phenyl isocyanate (0.100 g, 0.492 mmol) and tert-butyl (1R,4R,7S)-7-amino-2-azabicyclo[2.2.1]heptane-2-carboxylate (0.100 g, 0.471 mmol) via general method C. The final product was purified by silica gel chromatography (EtOAc:Hex/1:1). ¹H NMR (500 MHz, DMSO-d6) δ 8.67 (s, 1H), 7.52-7.42 (m, 2H), 7.22 (d, J=8.6 Hz, 2H), 6.37 (d, J=6.3 Hz, 1H), 3.76 (d, J=6.3 Hz, 1H), 3.15 (s, 1H), 2.93 (dt, J=9.5, 3.0 Hz, 1H), 2.52 (s, 1H), 2.15 (s, 1H), 1.76-1.63 (m, 2H), 1.48 (d, J=11.6 Hz, 1H), 1.38 (t, J=9.1 Hz, 1H).

### Example 44: MSU-43855/KSL-0007. 1-(3,3-difluoro-1-propionylpiperidin-4-yl)-3-(4-(trifluoromethoxy)phenyl)urea

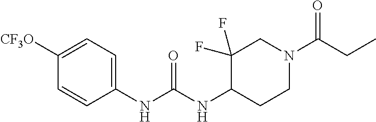

The title compound was prepared in 72% yield (20.06 mg) using 1-(3,3-difluoropiperidin-4-yl)-3-(4-(trifluoromethoxy)phenyl)urea (23.75 mg, 70.00 μmol) via general method G. ¹H NMR (500 MHz, DMSO-d6) δ 8.72 (d, J=2.7 Hz, 1H), 7.54-7.44 (m, 2H), 7.25 (d, J=8.6 Hz, 2H), 6.55 (d, J=9.2 Hz, 1H), 4.62 (t, J=11.6 Hz) & 4.34 (d, J=14.7 Hz, 2H), 4.18 (s) & 3.87 (d, J=14.0 Hz, 1H), 3.68-3.55 (m)&3.31-3.08 (m) & 2.85 (t, J=12.4 Hz, 2H), 2.44-2.25 (m, 2H), 1.95-1.82 (m, 1H), 1.59-1.46 & 1.44-1.33 (m, 1H), 0.98 (t, J=7.4 Hz, 3H). Purity: 95% Melting Point (° C.): 223-224 HRMS (found (ESI(+), [M−H]⁺): 396.1348

### Example 45: MSU-43864/KSL-0008. 1-((3S,4R)-3-fluoro-1-propionylpiperidin-4-yl)-3-(4-(trifluoromethoxy)phenyl)urea

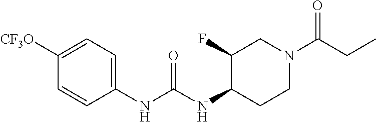

The title compound was prepared in 33% yield (55.45 mg) using 1-((3S,4R)-3-fluoropiperidin-4-yl)-3-(4-(trifluoromethoxy)phenyl)urea (142.60 mg, 443.85 μmol) via general method G. ¹H NMR (500 MHz, DMSO-d6) δ 8.72 (d, J=3.3 Hz, 1H), 7.50-7.44 (m, 2H), 7.23 (d, J=8.6 Hz, 2H), 6.41 (t, J=8.7 Hz, 1H), 4.82 (s) & 4.75-4.62 (m) & 4.43 (d, J=13.5 Hz, 2H), 4.18-4.08 (m) & 4.00-3.82 (m, 2H), 3.42 (d, J=15.1 Hz) & 3.12 (t, J=12.6 Hz, 1H), 2.87-2.78 (m) & 2.66 (dd, J=13.9, 11.1 Hz, 1H), 2.40-2.15 (m, 2H), 1.75-1.63 (m, 1H), 1.57 (qd, J=12.5, 4.3 Hz) & 1.42 (qd, J=12.7, 4.4 Hz, 1H), 0.98 (t, J=7.4 Hz, 3H). Purity: 95% Melting Point (° C.): 231-232 HRMS (found (ESI(+), [M−H]⁺): 378.1440

### Example 46: MSU-43865/KSL-0009. 1-((3R,4S)-3-fluoro-1-propionylpiperidin-4-yl)-3-(4-(trifluoromethoxy)phenyl)urea

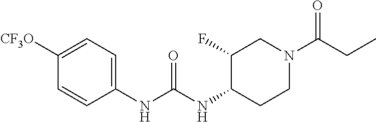

The title compound was prepared in 35% (61.89 mg) yield using 1-((3R,4S)-3-fluoropiperidin-4-yl)-3-(4-(trifluoromethoxy)phenyl)urea (151.85 mg, 472.64 μmol) via general method G. ¹H NMR (500 MHz, DMSO-d6) δ 8.70 (d, J=3.2 Hz, 1H), 7.49-7.45 (m, 2H), 7.29-7.21 (m, 2H), 6.41 (t, J=8.7 Hz, 1H), 4.82 (s) & 4.75-4.64 (m) & 4.43 (d, J=13.3 Hz, 2H), 4.13 (t, J=12.8 Hz) & 4.02-3.83 (m, 2H), 3.42 (d, J=15.1 Hz) & 3.13 (t, J=12.6 Hz, 1H), 2.95-2.79 (m) & 2.70-2.61 (m, 1H), 2.42-2.22 (m, 2H), 1.76-1.64 (m, 1H), 1.57 (qd, J=12.5, 4.3 Hz) & 1.42 (qd, J=12.6, 4.4 Hz, 1H), 0.98 (t, J=7.4 Hz, 3H). Purity: 95% Melting Point (° C.): 245-246 HRMS (found (ESI(+), [M−H]⁺): 378.1441

### Example 47: MSU-43866/KSL-0010. 1-((3S,4S)-3-fluoro-1-propionylpiperidin-4-yl)-3-(4-(trifluoromethoxy)phenyl)urea

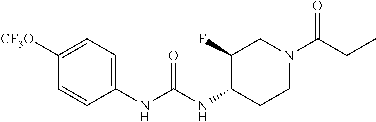

The title compound was prepared in 65% yield (29.09 mg) using 1-((3S,4S)-3-fluoropiperidin-4-yl)-3-(4-(trifluoromethoxy)phenyl)urea (38.02 mg, 118.34 μmol) via general method G. ¹H NMR (500 MHz, DMSO-d6) δ 8.69 (d, J=10.8 Hz, 1H), 7.53-7.46 (m, 2H), 7.23 (d, J=8.7 Hz, 2H), 6.54-6.42 (m, 1H), 4.42 (dp, J=12.7, 4.1 Hz) & 4.35-4.27 (m, 1H), 4.26-4.17 (m) & 3.95-3.84 (m, 2H), 3.79 (d, J=13.6 Hz) & 3.64 (d, J=14.0 Hz, 1H), 3.40 (dt, J 14.2, 7.3 Hz) & 3.30-3.09 (m, 2H), 2.35 (dt, J 9.2, 6.9 Hz, 2H), 1.97-1.79 (m, 1H), 1.48 (d, J=9.9 Hz) & 1.43-1.33 (m, 1H), 0.98 (t, J=7.4 Hz, 3H). Purity: 95% Melting Point (° C.): 180-181 HRMS (found (ESI(+), [M−H]⁺): 378.1442

### Example 48: MSU-43867/KSL-0011. 1-((2R,4S)-2-methyl-1-propionylpiperidin-4-yl)-3-(4-(trifluoromethoxy)phenyl)urea

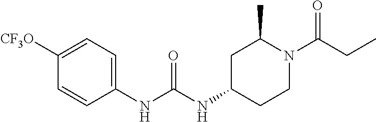

The title compound was prepared in 42% yield (47.99 mg) using 1-((2R,4S)-2-methylpiperidin-4-yl)-3-(4-(trifluoromethoxy)phenyl)urea (98.07 mg, 309.07 μmol) via general method G. ¹H NMR (500 MHz, DMSO-d6) δ 8.56 (s, 1H), 7.49-7.44 (m, 2H), 7.24-7.19 (m, 2H), 6.11 (d, J=7.7 Hz, 1H), 4.81 (s) & 4.37 (d, J=13.9 Hz, 1H), 4.25 (s) & 3.87 (s) & 3.72 (d, J=13.9 Hz, 2H), 3.15 (t, J=13.3 Hz) & 2.70 (t, J=13.3 Hz, 1H), 2.45-2.20 (m, 2H), 1.87 (s, 1H), 1.75 (t, J=16.8 Hz, 1H), 1.45 (d, J=5.3 Hz) & 1.32 (dt, J=12.4, 7.0 Hz, 1H), 1.26-1.14 (m, 2H), 1.09 (d, J=7.3 Hz, 2H), 0.98 (q, J=6.5 Hz, 3H). Purity: 95% Melting Point (° C.): 175-176 HRMS (found (ESI(+), [M−H]⁺): 374.1692

### Example 49: MSU-43868/KSL-0012. 1-((2R,4R)-2-methyl-1-propionylpiperidin-4-yl)-3-(4-(trifluoromethoxy)phenyl)urea

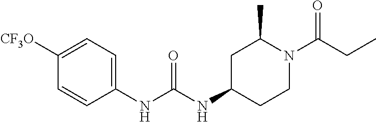

The title compound was prepared in 22% yield (38.71 mg) using 1-((2R,4R)-2-methylpiperidin-4-yl)-3-(4-(trifluoromethoxy)phenyl)urea (150.63 mg, 474.71 μmol) via general method G. ¹H NMR (500 MHz, DMSO-d6) δ 8.64 (s, 1H), 7.50-7.45 (m, 2H), 7.24-7.20 (m, 2H), 6.31 (d, J=6.6 Hz, 1H), 4.31 (s, 1H), 3.77 (q, J=5.1 Hz, 1H), 3.05 (s, 1H), 2.39-2.16 (m, 2H), 1.79 (q, J=5.5 Hz, 2H), 1.67 (d, J=13.8 Hz, 1H), 1.59 (d, J=13.2 Hz, 1H), 1.19 (d, J=6.9 Hz, 3H), 0.98 (t, J=7.3 Hz, 3H). Purity: 95%

### Example 50: MSU-43883/KSL-0027. 1-((1R,3s,5S)-8-propionyl-8-azabicyclo[3.2.1]octan-3-yl)-3-(4-(trifluoromethoxy)phenyl)urea

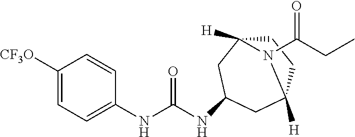

The title compound was prepared in 16% yield (16.83 mg) using 1-((1R,3s,5S)-8-azabicyclo[3.2.1]octan-3-yl)-3-(4-(trifluoromethoxy)phenyl)urea (89.84 mg, 272.80 μmol) via general method H. ¹H NMR (500 MHz, DMSO-d6) δ 8.53 (s, 1H), 7.50-7.43 (m, 2H), 7.24-7.17 (m, 2H), 6.10 (d, J=8.1 Hz, 1H), 4.48-4.43 (m, 1H), 4.26-4.22 (m, 1H), 4.04 (tq, J=12.1, 5.6 Hz, 1H), 2.35 (dq, J=16.0, 7.4 Hz, 1H), 2.25-2.16 (m, 1H), 1.98-1.86 (m, 2H), 1.77 (td, J=14.6, 8.2 Hz, 3H), 1.68 (t, J=10.6 Hz, 1H), 1.39 (td, J=12.1, 3.4 Hz, 2H), 1.00 (t, J 7.4 Hz, 3H). Melting Point (° C.): 166-168

### Example 51: MSU-43884/KSL-0028. 1-((1R,3r,5S)-8-propionyl-8-azabicyclo[3.2.1]octan-3-yl)-3-(4-(trifluoromethoxy)phenyl)urea

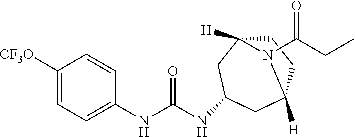

The title compound was prepared in 17.5% yield (20.09 mg) using 1-((1R,3r,5S)-8-azabicyclo[3.2.1]octan-3-yl)-3-(4-(trifluoromethoxy)phenyl)urea (98.37 mg, 298.71 μmol) via general method H. ¹H NMR (500 MHz, DMSO-d6) δ 8.71 (s, 1H), 7.50-7.44 (m, 2H), 7.26-7.19 (m, 2H), 6.43 (d, J=6.5 Hz, 1H), 4.45-4.41 (m, 1H), 4.21 (s, 1H), 3.86 (q, J=6.5 Hz, 1H), 2.32 (dq, J=15.0, 7.4 Hz, 1H), 2.24-2.15 (m, 1H), 2.06-1.92 (m, 5H), 1.83 (dq, J=12.4, 7.4 Hz, 1H), 1.71 (d, J=14.5 Hz, 1H), 1.62 (d, J=14.4 Hz, 1H), 0.99 (t, J=7.5 Hz, 3H). Melting Point (° C.): 204-205

### Example 52: MSU-43881/KSL-0025. 6-propionamido-N-(4-(trifluoromethoxy)phenyl)-2-azaspiro[3.3]heptane-2-carboxamide

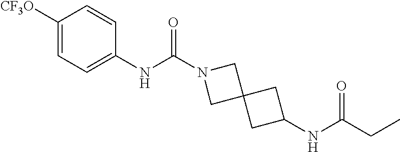

The title compound was prepared in 2.9% yield (2.87 mg) using 6-amino-N-(4-(trifluoromethoxy)phenyl)-2-azaspiro[3.3]heptane-2-carboxamide (84.81 mg, 268.98 μmol) via general method H. ¹H NMR (500 MHz, DMSO-d6) δ 8.58 (s, 1H), 7.98 (d, J=7.4 Hz, 1H), 7.61-7.55 (m, 2H), 7.22 (d, J=8.7 Hz, 2H), 4.05 (h, J=8.1 Hz, 1H), 3.98 (s, 2H), 3.87 (s, 2H), 2.44 (ddd, J=10.0, 7.7, 3.0 Hz, 2H), 2.08-1.99 (m, 4H), 0.96 (t, J=7.6 Hz, 3H). Yield: 2.87 mg, 2.87% Melting Point (° C.): 234-236

### Example 53: MSU-43878/KSL-0022. 1-(2-propionyl-2-azaspiro[3.3]heptan-6-yl)-3-(4-(trifluoromethoxy)phenyl)urea

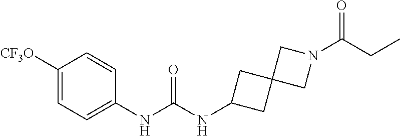

The title compound was prepared in 0.9% yield (1.16 mg) using 1-(2-azaspiro[3.3]heptan-6-yl)-3-(4-(trifluoromethoxy)phenyl)urea (114.14 mg, 362.00 μmol) via general method H. ¹H NMR (500 MHz, DMSO-d6) δ 8.58 (s, 1H), 7.49-7.43 (m, 2H), 7.21 (d, J=8.7 Hz, 2H), 6.46 (d, J=7.8 Hz, 1H), 4.13 (s, 1H), 4.01 (d, J=10.8 Hz, 2H), 3.86 (s, 1H), 3.75 (s, 1H), 2.48-2.43 (m, 2H), 2.08-1.97 (m, 4H), 0.93 (td, J=7.5, 2.6 Hz, 3H). Melting Point (° C.): 196-198

### Example 54: MSU-43875/KSL-0019. 1-((1R,5S,6s)-3-propionyl-3-azabicyclo[3.1.0]hexan-6-yl)-3-(4-(trifluoromethoxy)phenyl)urea

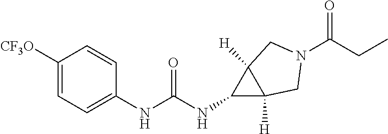

The title compound was prepared in 13.2% (18.13 mg) yield using 1-((1R,5S,6s)-3-azabicyclo[3.1.0]hexan-6-yl)-3-(4-(trifluoromethoxy)phenyl)urea (116.21 mg, 385.73 μmol) via general method H. ¹H NMR (500 MHz, DMSO-d6) δ 8.59 (s, 1H), 7.53-7.47 (m, 2H), 7.25-7.20 (m, 2H), 6.52 (d, J=2.7 Hz, 1H), 3.63 (dd, J=11.0, 6.0 Hz, 2H), 3.54 (dd, J=10.3, 4.7 Hz, 1H), 3.30 (dd, J=11.8, 4.8 Hz, 1H), 2.27-2.11 (m, 3H), 1.76 (ddd, J=7.1, 4.6, 2.2 Hz, 1H), 1.67 (ddd, J=7.5, 4.7, 2.2 Hz, 1H), 0.94 (t, J=7.4 Hz, 3H). Melting Point (° C.): 146-148

### Example 55: MSU43476/MBG-VII-10 tert-Butyl {1-[(2,3,4-trifluorophenyl)carbamoyl]azetidin-3-yl}carbamate

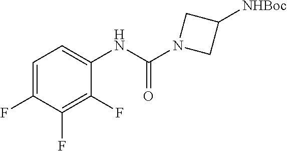 [SMILES: CC(C)(C)OC(=O)NC1CN(C(=O)Nc2ccc(F)c(F)c2F)C1]

The title compound was prepared from 2,3,4-trifluorophenylisocyanate (0.432 g, 2.50 mmol) and tert-Butyl azetidine-3-yl-carbamate (0.447 g, 2.60 mmol) by method A in 31% yield (0.268 g). On combining the reagents, a solid was formed. The product was purified by filtration and washing with hexanes. ¹H NMR (500 MHz, DMSO-d6) δ 8.42 (s, 1H), 7.55 (d, J=7.3 Hz, 1H), 7.35-7.17 (m, 2H), 4.26 (q, J=6.9 Hz, 1H), 4.12 (t, J=8.1 Hz, 2H), 3.77 (dd, J=8.5, 5.5 Hz, 2H), 1.37 (s, 9H). 19F NMR (470 MHz, DMSO-d6) δ −141.17 (ddt, J=22.3, 9.5, 4.2 Hz), 142.80 (dd, J=21.5, 8.1 Hz), −161.18 (td, J=21.6, 8.1 Hz). HRMS ESI (+) [M+Na] calc'd=368.1198, found=368.1206

### Example 56: MSU-43475/MBG-VII-74. N-{[1-(2-methylpropanoyl)pyrrolidin-3-yl]methyl}-N-[4-(trifluoromethoxy)phenyl]urea

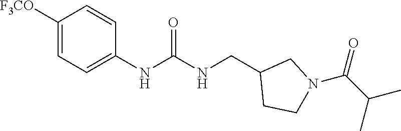 [SMILES: CC(C)C(=O)N1CCC(CNC(=O)Nc2ccc(OC(F)(F)F)cc2)C1]

Using general method B, the title compound was prepared by deprotection of tert-butyl 3-[({[4(trifluoromethoxy)phenyl]carbamoyl}amino)methyl]pyrrolidine-1-carboxylate (1.22 g, 3.0 mmol) with trifluoroacetic acid (3.00 mL) in dichloromethane (15.0 mL). The mixture was then concentrated in vacuo. The residue was used, without further purification to produce the title compound (24% yield, 0.17 g) when mixed with isobutyryl chloride (0.424 g, 4.00 mmol) and triethylamine (0.800 mL, 7.90 mmol) in dichloromethane (8.00 mL). The product was subsequently purified by reverse phase medium pressure liquid chromatography (25 mM aqueous ammonium formate/methanol (0 to 100%)) and then by normal phase medium pressure liquid chromatography (SiO₂, dichloromethane to 5% methanol/dichloromethane) to provide a solid. ¹H NMR (500 MHz, DMSO-d6) δ 8.63 (d, J=16.7 Hz, 1H), 7.50-7.44 (m, 2H), 7.20 (ddd, J=9.1, 2.3, 1.1 Hz, 2H), 6.36 (q, J=5.7 Hz, 1H), 3.56 (ddd, J=11.4, 7.5, 4.0 Hz, 1H), 3.45-3.37 (m, 1H), 3.25-3.02 (m, 3H), 3.00-2.94 (m, 1H), 2.61 (dhept, J=10.1, 6.7 Hz, 1H), 2.31 (ddt, J=57.7, 14.4, 7.1 Hz, 1H), 2.00-1.82 (m, 1H), 1.58 (ddq, J=54.6, 12.4, 7.9 Hz, 1H), 0.99-0.94 (m, 6H). 19F NMR (470 MHz, DMSO-d6) δ −57.16. HRMS ESI (+) calc'd for [M+H]=374.1687, found=374.1715

### Example 57: MSU-43879/KSL-0023. 1-((1R,5S,6r)-3-propionyl-3-azabicyclo[3.1.0]hexan-6-yl)-3-(4-(trifluoromethoxy)phenyl)urea

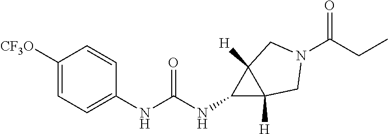

The title compound was prepared in 80.65% (103.43 mg) yield using 1-((1R,5S,6r)-3-azabicyclo[3.1.0]hexan-6-yl)-3-(4-(trifluoromethoxy)phenyl)urea (110.48 mg, 366.71 μmol) via general method H. ¹H NMR (500 MHz, dmso) δ 8.61 (d, J=17.0 Hz, 1H), 7.53-7.46 (m, 2H), 7.26-7.19 (m, 2H), 6.53 (d, J=9.8 Hz, 1H), 3.77-3.60 (m, 2H), 3.60-3.51 (m, 1H), 2.48-2.40 (m, 1H), 2.25 (dq, J=7.4, 2.5 Hz, 1H), 2.22-2.11 (m, 1H), 1.82-1.65 (m, 2H), 1.12 (dd, J=6.9, 5.7 Hz, 1H), 1.00-0.88 (m, 3H).

### Example 58: MSU-43998/KSL-0035. 1-((1R,4R,5S)-2-propionyl-2-azabicyclo[2.1.1]hexan-5-yl)-3-(4-(trifluoromethoxy)phenyl)urea

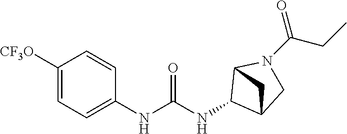

The title compound was prepared in 90.46% (155.70 mg) yield using 1-((1R,4R,5S)-2-azabicyclo[2.1.1]hexan-5-yl)-3-(4-(trifluoromethoxy)phenyl)urea (160.53 mg, 532.84 μmol) via general method H. ¹H NMR (500 MHz, dmso) δ 8.75-8.60 (m, 1H), 7.48-7.40 (m, 2H), 7.25-7.18 (m, 2H), 6.37-6.22 (m, 1H), 4.52-4.35 (m, 1H), 3.95-3.83 (m, 1H), 3.23 (dd, J=9.2, 4.7 Hz, 1H), 3.15 (d, J 9.9 Hz, 1H), 2.85-2.74 (m, 1H), 2.37-2.16 (m, 2H), 1.69-1.57 (m, 1H), 1.19-1.08 (m, 1H), 1.07-0.96 (m, 3H).

### Example 59: MSU-43999/KSL-0036. 1-((1R,4R,7S)-2-propionyl-2-azabicyclo[2.2.1]heptan-7-yl)-3-(4-(trifluoromethoxy)phenyl)urea

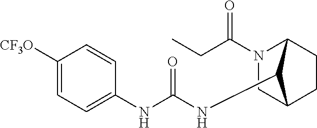

The title compound was prepared in 162.37% (264.07 mg) yield using 1-((1R,4R,7S)-2-azabicyclo[2.2.1]heptan-7-yl)-3-(4-(trifluoromethoxy)phenyl)urea (87.22 mg, 276.63 μmol) via general method H. 1H NMR (500 MHz, dmso) δ 8.61 (d, J=4.8 Hz, 1H), 7.51-7.44 (m, 2H), 7.26-7.18 (m, 2H), 6.48-6.38 (m, 1H), 4.28-4.06 (m, 1H), 3.81-3.71 (m, 1H), 3.52-3.47 (m, 1H), 3.26 (d, J=3.2 Hz, 1H), 3.12-2.94 (m, 1H), 2.46-2.34 (m, 1H), 2.33-2.09 (m, 2H), 1.85-1.66 (m, 2H), 1.46-1.37 (m, 1H), 1.16-1.03 (m, 1H), 0.97 (dt, J=12.8, 7.4 Hz, 3H).

### Example 60: MSU-45007/MBG XI-28. 3-[(2,2,3,3,3-pentafluoropropanamido)methyl]-N-[4-(trifluoromethyl)phenyl]pyrrolidine-1-carboxamide

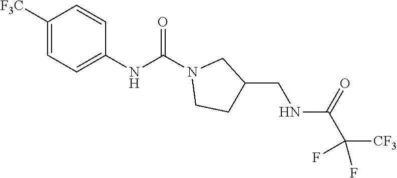 [SMILES: O=C(Nc1ccc(C(F)(F)F)cc1)N1CCC(CNC(=O)C(F)(F)C(F)(F)F)C1]

Using general method B, tert-butyl N-[(1-{[4-(trifluoromethyl)phenyl]carbamoyl}pyrrolidin-3-yl)methyl]carbamate (0.317 g, 0.924 mmol) was deprotected with trifluoroacetic acid (2.70 g, 23.7 mmol) in dichloromethane (4.00 mL) stirring in an argon atmosphere for 20 hours. The crude material was concentrated in vacuo to constant weight and dissolved in dichloromethane under an argon atmosphere (10.0 mL), triethylamine (0.707 g, 7.00 mmol) was added, followed by pentafluoropropionic anhydride (0.310 g, 1.00 mmol). The reaction mixture was stirred for 20 hours, concentrated in vacuo, purified by silica gel chromatography (0 to 10% methanol in dichloromethane) followed by reverse phase medium pressure liquid chromatography (0 to 100% methanol in 25 mM aqueous ammonium formate). Fractions containing product were concentrated, dissolved in ethyl acetate, washed with saturated aqueous sodium bicarbonate, brine, dried over sodium sulfate, filtered, and concentrated in vacuo to yield the product as a solid (0.196 g, 49%). ¹H NMR (500 MHz, dmso-d₆) δ 9.72-9.53 (m, 1H), 8.52 (s, 1H), 7.72 (d, J=8.5 Hz, 2H), 7.56 (d, J=8.6 Hz, 2H), 3.56-3.45 (m, 2H), 3.37 (dt, J=10.2, 7.4 Hz, 1H), 3.24 (tq, J=11.7, 6.6 Hz, 2H), 3.10 (dd, J=10.6, 6.7 Hz, 1H), 2.44 (t, J=7.4 Hz, 1H), 1.96 (h, J=5.7 Hz, 1H), 1.68-1.57 (m, 1H). ¹⁹F NMR (470 MHz, dmso-d₆) δ −59.99, −82.37, −121.85. HRMS ESI (+) calc'd for [M+H]=434.1111, observed=434.1126.

### Example 61: MSU-45008/MBG-XI-35. tert-Butyl (3a,6a)-5-{[4-(trifluoromethoxy)phenyl]carbamoyl}-octahydropyrrolo[3,4-c]pyrrole-2-carboxylate

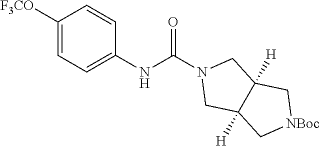 [SMILES: CC(C)(C)OC(=O)N1C[C@H]2CN(C(=O)Nc3ccc(OC(F)(F)F)cc3)C[C@H]2C1]

Using general method A, 4-trifluoromethoxyphenyl isocyanate (0.812 g, 4.00 mmol) was dissolved in dichloromethane (6.00 mL) in a sealed vessel under an argon atmosphere. Tert-butyl hexahydropyrrolo[3,4c]pyrrole-2(1H)-carboxylate (0.891 g, 4.20 mmol) was dissolved in dichloromethane (4.00 mL) and added to the isocyanate solution by syringe. The reaction mixture was stirred for 18 hours, concentrated in vacuo, and purified by silica gel chromatography (0 to 10% methanol in dichloromethane) to yield the product as a white powder (1.508 g 90% yield). ¹H NMR (500 MHz, dmso-d₆) δ 8.34 (s, 1H), 7.63-7.50 (m, 2H), 7.22 (d, J=8.6 Hz, 2H), 3.60 (q, J=9.6 Hz, 2H), 3.47 (d, J=16.0 Hz, 2H), 3.31-3.21 (m, 2H), 3.17-3.08 (m, 2H), 2.88 (s, 2H), 1.39 (s, 9H). ¹⁹F NMR (470 MHz, dmso-d₆) δ-57.10. HRMS ESI (+) calc'd for [M+Na]=438.1617, observed=438.1635.

### Example 62: MSU-45009/MBG XI-37. tert-Butyl (3a,6a)-5-{[4-(trifluoromethyl)phenyl]carbamoyl}-octahydropyrrolo[3,4-c]pyrrole-2-carboxylate

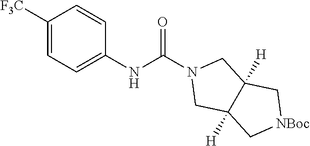 [SMILES: CC(C)(C)OC(=O)N1C[C@H]2CN(C(=O)Nc3ccc(C(F)(F)F)cc3)C[C@H]2C1]

Using general method A, 4-trifluoromethylphenyl isocyanate (0.692 g, 3.70 mmol) was dissolved in dichloromethane (5.00 mL) in a sealed vessel under an argon atmosphere. tert-Butyl hexahydropyrrolo[3,4c]pyrrole-2(1H)-carboxylate (0.827 g, 3.89 mmol) was dissolved in dichloromethane (4.00 mL) and added to the isocyanate solution by syringe. The reaction mixture was stirred for 18 hours and concentrated in vacuo, during which a white solid precipitated. The reaction mixture was filtered, washed with hexanes, and dried in vacuo to yield the pure product as a white solid (0.755 g, 51%). ¹H NMR (500 MHz, dmso-d₆) δ 8.52 (s, 1H), 7.71 (d, J=8.4 Hz, 2H), 7.56 (d, J=8.5 Hz, 2H), 3.61 (d, J=8.9 Hz, 2H), 3.52-3.41 (m, 2H), 3.28 (s, 2H), 3.12 (s, 2H), 2.88 (s, 2H), 1.38 (s, 9H). ¹⁹F NMR (470 MHz, dmso-d₆) δ −59.99. HRMS ESI (+) calc'd for [M+Na]=422.1668, observed=422.1686.

### Example 63: MSU-45010/MBG-XI-38. tert-Butyl 7-{[4-(trifluoromethoxy)phenyl]carbamoyl}-2,7-diazaspiro[4.4]nonane-2-carboxylate

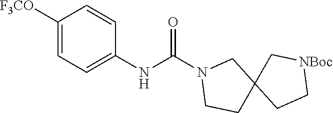 [SMILES: CC(C)(C)OC(=O)N1CCC2(CCN(C(=O)Nc3ccc(OC(F)(F)F)cc3)C2)C1]

Using general method A, 4-trifluoromethoxyphenyl isocyanate (0.893 g, 4.40 mmol) was dissolved in dichloromethane (10.0 mL) in a sealed vessel under an argon atmosphere. tert-Butyl-2,7-diazaspiro[4,4]nonane-2-carboxylate (1.03 g, 4.57 mmol) was dissolved in dichloromethane (5.00 mL) and added to the isocyanate solution by syringe. The reaction mixture was stirred for 18 hours, concentrated in vacuo, and purified by silica gel chromatography (0 to 10% methanol in dichloromethane) to yield the product which triturated on concentration. The solid was filtered, washed with hexanes, and dried in vacuo to yield the pure product as a white solid (0.860 g, 45%). ¹H NMR (500 MHz, dmso-d₆) δ 8.32 (s, 1H), 7.67-7.54 (m, 2H), 7.21 (d, J=8.7 Hz, 2H), 3.44 (q, J=6.4 Hz, 2H), 3.39-3.27 (m, 4H), 3.27-3.11 (m, 2H), 1.90-1.72 (m, 4H), 1.38 (d, J=3.1 Hz, 9H). ¹⁹F NMR (470 MHz, dmso-d₆) δ 59.99. HRMS ESI (+) calc'd for [M+Na]=452.1773, observed=452.1786.

### Example 64: MSU-45011/MBG-XI-39. tert-Butyl 7-{[4-(trifluoromethoxy)phenyl]carbamoyl}-2,7-diazaspiro[3.5]nonane-2-carboxylate

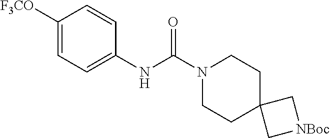 [SMILES: CC(C)(C)OC(=O)N1CC2(CCN(C(=O)Nc3ccc(OC(F)(F)F)cc3)CC2)C1]

Using general method A, 4-trifluoromethoxyphenyl isocyanate (0.934 g, 4.60 mmol) was dissolved in dichloromethane (10.0 mL) in a sealed vessel under an argon atmosphere. 2-(tert-Butoxycarbonyl)-2,7-diazaspiro[3,5]nonane (1.08 g, 4.79 mmol) was dissolved in dichloromethane (5.00 mL) and added to the isocyanate solution by syringe. The reaction mixture was stirred for 18 hours, concentrated in vacuo, and purified by silica gel chromatography (0 to 10% methanol in dichloromethane) to yield the product which triturated on concentration. The solid was filtered, washed with hexanes, and dried in vacuo to yield the pure product as a white solid (1.29 g, 65%). ¹H NMR (500 MHz, dmso-d₆) δ 8.69 (s, 1H), 7.59-7.44 (m, 2H), 7.21 (d, J=8.6 Hz, 2H), 3.67-3.51 (m, 4H), 3.37 (d, J=17.2 Hz, 4H), 1.65 (t, J=5.6 Hz, 4H), 1.37 (s, 9H). ¹⁹F NMR (470 MHz, dmso-d₆) δ −57.10. HRMS ESI (+) calc'd for [M+Na]=452.1773, observed=452.1787.

### Example 65: MBG-XI-46. tert-Butyl 3-(propoxymethyl)pyrrolidine-1-carboxylate (intermediate)

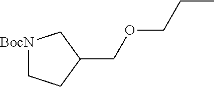 [SMILES: CCCOCC1CCN(C(=O)OC(C)(C)C)C1]

1-Boc-3-hydroxymethylpyrrolidine (1.09 g, 5.44 mmol) was added to a 100 mL 24/40 roundbottom flask, which was sealed and flushed with argon. N,N-dimethylformamide (12.0 mL) was added by syringe and the vessel was cooled to 0° C. in an ice-water bath. Sodium hydride (3.02 g, 9.00 mmol) was added as a solid. The reaction mixture was resealed, flushed with argon, and stirred at 0° C. for 5 minutes, at which point n-propyl bromide (1.35 g, 11.0 mmol) was added via syringe. The reaction mixture was heated to 55° C. for 22 hours, concentrated in vacuo, partitioned between ethyl acetate and saturated aqueous ammonium chloride. The organic layer was washed with water, brine, dried over sodium sulfate, filtered, concentrated in vacuo and purified by silica gel chromatography (0 to 100% ethyl acetate in hexanes) to yield the pure product as an oil (0.610 g, 46%). ¹H NMR (500 MHz, CDCl₃) δ 3.54-3.23 (m, 7H), 3.12-3.00 (m, 1H), 2.45 (h, J=7.4 Hz, 1H), 1.95 (p, J=5.7 Hz, 1H), 1.67 (ddd, J=16.5, 9.9, 7.8 Hz, 1H), 1.58 (p, J=7.1 Hz, 2H), 1.45 (s, 9H), 0.91 (td, J=7.5, 2.9 Hz, 3H).

### Example 66: MSU-45070/MBG-XI-48. 3-(Propoxymethyl)-N-[4-(trifluoromethoxy)phenyl]pyrrolidine-1-carboxamide

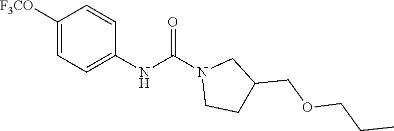 [SMILES: CCCOCC1CCN(C(=O)Nc2ccc(OC(F)(F)F)cc2)C1]

tert-Butyl 3-(propoxymethyl)pyrrolidine-1-carboxylate produced above in Example 65 was dissolved in dichloromethane (8.00 mL) and deprotected by adding trifluoroacetic acid (4.00 mL, 23.7 mmol) and stirred for 18 hours. The mixture was concentrated in vacuo and dried to a constant weight. The TFA salt was dissolved in dichloromethane (3.00 mL), triethylamine was added (0.404 g, 4.00 mmol) and the freebase was used in preparation of 3-(propoxymethyl)-N-[4-(trifluoromethoxy)phenyl]pyrrolidine-1-carboxamide. Using general method A, 4-trifluoromethoxyphenyl isocyanate (0.304 g, 1.50 mmol) was dissolved in dichloromethane (3.00 mL) in a sealed vessel under an argon atmosphere. Crude 3-(propoxymethyl)pyrrolidine (0.590 g, 4.12 mmol) was dissolved in dichloromethane (3.00 mL) and triethylamine (0.404 g, 4.00 mmol) and added to the isocyanate solution by syringe. The mixture was stirred for 18 hours, concentrated in vacuo, and purified by silica gel chromatography (0 to 10% methanol in dichloromethane) to yield the pure product (0.293 g, 56% yield). ¹H NMR (500 MHz, cdc13) δ 7.47-7.41 (m, 2H), 7.14 (d, J=8.6 Hz, 2H), 6.21 (s, 1H), 3.66-3.56 (m, 2H), 3.47 (dt, J=9.5, 7.1 Hz, 2H), 3.43-3.36 (m, 3H), 3.26 (dd, J=9.6, 6.8 Hz, 1H), 2.61 (hept, J=7.1 Hz, 1H), 2.13-2.05 (m, 1H), 1.80 (dq, J=12.7, 8.0 Hz, 1H), 1.60 (h, J=7.1 Hz, 2H), 0.93 (t, J=7.4 Hz, 3H). ¹⁹F NMR (470 MHz, cdc13) δ −58.19. HRMS ESI (+) calc'd for [M+H]=347.1578, found=347.1613.

### Example 67: MSU-45086/MBG-XI-56. tert-Butyl 7-{[4-(trifluoromethyl)phenyl]carbamoyl}-2,7-diazaspiro[3.5]nonane-2-carboxylate

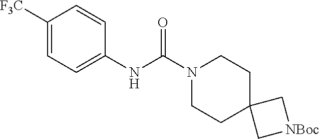 [SMILES: CC(C)(C)OC(=O)N1CC2(CCN(C(=O)Nc3ccc(C(F)(F)F)cc3)CC2)C1]

Using general method A, 4-trifluoromethylphenyl isocyanate (0.430 g, 2.30 mmol) was dissolved in dichloromethane (8.00 mL) in a sealed vessel under an argon atmosphere. 2-(tert-Butoxycarbonyl)-2,7-diazaspiro[3,5]nonane (0.565 g, 2.50 mmol) was dissolved in dichloromethane (5.00 mL) and added to the isocyanate solution by syringe. The reaction mixture was stirred for 18 hours during which time a solid precipitated. The solid was filtered, washed with hexanes and dried in vacuo to yield the pure product (0.735 g, 77%). ¹H NMR (500 MHz, dmso-d₆) δ 8.88 (s, 1H), 7.66 (d, J=8.6 Hz, 2H), 7.55 (d, J=8.7 Hz, 2H), 3.64-3.50 (m, 4H), 3.47-3.34 (m, 4H), 1.65 (t, J=5.6 Hz, 4H), 1.36 (s, 9H). ¹⁹F NMR (470 MHz, dmso-d₆) δ −60.01. HRMS ESI (+) calc'd for [M+Na]=436.1824, found=436.1837.

### Example 68: MSU-45087/MB G XI-57. tert-butyl 7-{[4-(trifluoromethyl)phenyl]carbamoyl}-2,7-diazaspiro[4.4]nonane-2-carboxylate

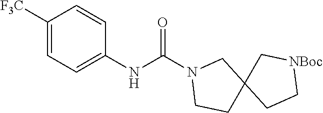 [SMILES: CC(C)(C)OC(=O)N1CCC2(CCN(C(=O)Nc3ccc(C(F)(F)F)cc3)C2)C1]

Using general method A, 4-trifluoromethylphenyl isocyanate (0.710 g, 3.80 mmol) was dissolved in dichloromethane (5.00 mL) in a sealed vessel under an argon atmosphere. tert-Butyl-2,7-diazaspiro[4,4]nonane-2-carboxylate (0.930 g, 4.10 mmol) was dissolved in dichloromethane (5.00 mL) and added to the isocyanate solution by syringe. The reaction mixture was stirred for 18 hours, concentrated in vacuo and purified by silica gel chromatography (0 to 10% methanol in dichloromethane) to yield the pure product as a white solid (1.56 g, 99%). ¹H NMR (500 MHz, dmso-d₆) δ 8.51 (s, 1H), 7.73 (d, J=8.6 Hz, 2H), 7.56 (d, J=8.6 Hz, 2H), 3.47 (t, J=7.0 Hz, 2H), 3.34 (d, J=5.4 Hz, 2H), 3.28 (d, J=7.6 Hz, 2H), 3.24-3.14 (m, 2H), 1.94-1.73 (m, 4H), 1.38 (d, J=3.7 Hz, 9H). ¹⁹F NMR (470 MHz, dmso-d₆) δ −59.98. HRMS ESI (+) calc'd for [M+Na]=436.1824, found=436.1849.

### Example 69: MSU-45090/MBG-XI-86. tert-Butyl N-(1-{[4 (trifluoromethyl)phenyl]carbamoyl}piperidin-4-yl)carbamate

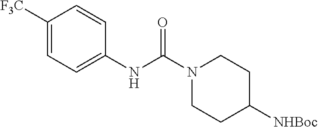 [SMILES: CC(C)(C)OC(=O)NC1CCN(C(=O)Nc2ccc(C(F)(F)F)cc2)CC1]

Using general method A, 4-trifluoromethylphenyl isocyanate (0.710 g, 3.80 mmol) was dissolved in dichloromethane (2.00 mL) in a sealed vessel under an argon atmosphere. Tert-Butyl piperidin-4-ylcarbamate (0.801, 4.00 mmol) was dissolved in dichloromethane (9.00 mL) and added to the isocyanate solution by syringe. The reaction mixture was stirred for 20 hours during which time a precipitate formed. The solid was filtered, washed with hexanes, and dried in vacuo to yield the pure product (1.21 g, 82%). 1H NMR (500 MHz, dmso-d₆) δ 8.88 (s, 1H), 7.66 (d, J=8.5 Hz, 2H), 7.56 (d, J=8.8 Hz, 2H), 6.88 (d, J=7.9 Hz, 1H), 4.02 (d, J=13.5 Hz, 2H), 3.46 (s, 1H), 2.88 (td, J=12.7, 2.7 Hz, 2H), 1.78-1.67 (m, 2H), 1.38 (s, 9H), 1.33-1.22 (m, 2H). ¹⁹F NMR (470 MHz, dmso-d₆) δ −59.98. HRMS ESI (+) Calc'd for [M+Na]=410.1668, observed=410.1684.

### Example 70: MSU-45106/MB G XI-87. tert-Butyl N-(1-{[4-(trifluoromethyl)phenyl]carbamoyl}piperidin-3-yl)carbamate

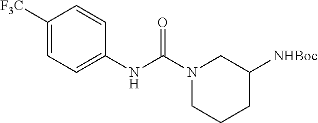 [SMILES: CC(C)(C)OC(=O)NC1CCCN(C(=O)Nc2ccc(C(F)(F)F)cc2)C1]

Using general method A, 4-trifluoromethylphenyl isocyanate (0.710 g, 3.80 mmol) was dissolved in dichloromethane (2.00 mL) in a sealed vessel under an argon atmosphere. tert-Butyl piperidin-3-ylcarbamate (0.801, 4.00 mmol) was dissolved in dichloromethane (10.0 mL) and added to the isocyanate solution by syringe. The reaction mixture was stirred for 20 hours during which time a precipitate formed. The solid was filtered, washed with hexanes, and dried in vacuo to yield the pure product (1.08 g, 73%). ¹H NMR (500 MHz, dmso-d₆) δ 8.89 (s, 1H), 7.69-7.62 (m, 2H), 7.56 (d, J=8.7 Hz, 2H), 6.91 (d, J=7.8 Hz, 1H), 3.95 (d, J=12.4 Hz, 1H), 3.85 (d, J=13.4 Hz, 1H), 2.85 (t, J=11.5 Hz, 1H), 2.78-2.66 (m, 1H), 1.81 (d, J=11.1 Hz, 1H), 1.70 (d, J=11.4 Hz, 1H), 1.36 (s, 12H). ¹⁹F NMR (470 MHz, dmso-d₆) δ −59.97. HRMS ESI (+) Calc'd for [M+Na]=410.1668, observed=410.1686.

### Example 71: MSU-45109/MBG-XI-93. tert-Butyl N-[(3-methyl-1-{[4-(trifluoromethyl)phenyl]carbamoyl}pyrrolidin-3-yl)methyl]carbamate

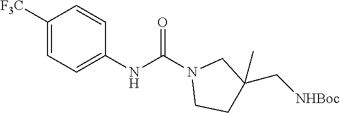 [SMILES: CC1(CNC(=O)OC(C)(C)C)CCN(C(=O)Nc2ccc(C(F)(F)F)cc2)C1]

Using general method A, 4-trifluoromethylphenyl isocyanate (0.196 g, 1.05 mmol) was dissolved in dichloromethane (6.00 mL) in a sealed vessel under an argon atmosphere. tert-Butyl ((3-methylpyrrolidin-3-yl)methyl)carbamate (0.255 g, 1.18 mmol) was dissolved in dichloromethane (4.00 mL) and added to the isocyanate solution by syringe. The reaction mixture stirred for 22 hours, was concentrated in vacuo, and purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product as a white solid (0.300 g, 71%). ¹H NMR (500 MHz, dmso-d₆) δ 8.46 (s, 1H), 7.73 (d, J=8.5 Hz, 2H), 7.55 (d, J=8.7 Hz, 2H), 7.04 (t, J=6.4 Hz, 1H), 3.52-3.38 (m, 2H), 3.23 (d, J=10.5 Hz, 1H), 3.06 (d, J=10.4 Hz, 1H), 2.97 (dd, J=13.7, 6.5 Hz, 1H), 2.88 (dd, J=13.7, 6.2 Hz, 1H), 1.77 (dt, J=13.5, 7.1 Hz, 1H), 1.53 (dt, J=13.1, 7.3 Hz, 1H), 1.36 (s, 9H), 0.97 (s, 3H). ¹⁹F NMR (470 MHz, dmso-d₆) δ −59.96. HRMS ESI (+) calc'd for [M+H]=402.2000, observed=402.2026.

### Example 72: MSU-45150/MBG XI-99. tert-Butyl N-[(1-{[4-(trifluoromethyl)phenyl]carbamoyl}piperidin-3-yl)methyl]carbamate

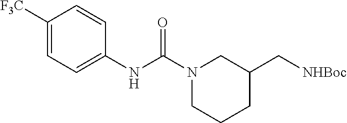 [SMILES: CC(C)(C)OC(=O)NCC1CCCN(C(=O)Nc2ccc(C(F)(F)F)cc2)C1]

Using general method A, 4-trifluoromethylphenyl isocyanate (0.710 g, 3.80 mmol) was dissolved in dichloromethane (5.00 mL) in a sealed vessel under an argon atmosphere. tert-Butyl N-(piperidin-3-yl)methylcarbamate (0.801, 4.00 mmol) was dissolved in dichloromethane (10.0 mL) and added to the isocyanate solution by syringe. The reaction mixture was stirred for 18 hours during which time a precipitate formed. The solid was filtered, washed with hexanes, and dried in vacuo to yield the pure product (1.59 g, 99%). ¹H NMR (500 MHz, cd₃od) δ 7.62-7.35 (m, 4H), 4.01 (dd, J=13.3, 3.7 Hz, 1H), 3.94 (dd, J=13.4, 4.4 Hz, 1H), 3.00 (pd, J=12.2, 6.7 Hz, 3H), 2.75 (dd, J=13.3, 9.9 Hz, 1H), 1.85 (dt, J=13.0, 4.2 Hz, 1H), 1.82-1.65 (m, 2H), 1.53 (ddd, J=15.1, 12.2, 8.8 Hz, 1H), 1.43 (s, 9H), 1.27 (dtd, J=14.4, 10.7, 3.7 Hz, 1H). ¹⁹F NMR (470 MHz, cd₃od) δ −63.35. HRMS ESI (+) calc'd for [M+H]=402.2000, observed=402.2012.

### Example 73: MSU-45151/MBG-XI-100. tert-Butyl 2-{[4-(trifluoromethyl)phenyl]carbamoyl}-2,7-diazaspiro[4.5]decane-7-carboxylate

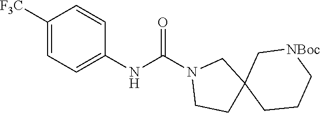 [SMILES: CC(C)(C)OC(=O)N1CCCC2(CCN(C(=O)Nc3ccc(C(F)(F)F)cc3)C2)C1]

Using general method A, 4-trifluoromethylphenyl isocyanate (0.168 g, 0.897 mmol) was dissolved in dichloromethane (3.00 mL) in a sealed vessel under an argon atmosphere. tert-Butyl-2,7-diazaspiro[4,5]decane-7-carboxylate (0.225, 0.937 mmol) was dissolved in dichloromethane (3.0 mL) and added to the isocyanate solution by syringe. The reaction was stirred for 19 hours, concentrated in vacuo and purified by silica gel chromatography (0 to 10% methanol in dichloromethane) to yield the pure product (0.380 g, 95%). ¹H NMR (500 MHz, CDCl₃) δ 7.53 (s, 4H), 6.33 (s, 1H), 3.58 (s, 2H), 3.50-3.20 (m, 5H), 3.15 (d, J=10.0 Hz, 1H), 1.94 (s, 1H), 1.79-1.51 (m, 5H), 1.44 (d, J=4.8 Hz, 9H). ¹⁹F NMR (HRMS ESI (+) Calc'd for [M+Na]=366.1406, observed=366.1425.470 MHz, CDCl₃) δ −61.88. HRMS ESI (+) calc'd for [M+H]=428.2157, observed=428.2173.

### Example 74: MSU-45156/MBG-XI-108. tert-Butyl N-[(1-{[4-(trifluoromethyl)phenyl]carbamoyl}piperidin-4-yl)methyl]carbamate

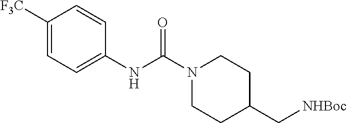 [SMILES: CC(C)(C)OC(=O)NCC1CCN(C(=O)Nc2ccc(C(F)(F)F)cc2)CC1]

Using general method A, 4-trifluoromethylphenyl isocyanate (0.841 g, 4.50 mmol) was dissolved in dichloromethane (5.00 mL) in a sealed vessel under an argon atmosphere. tert-Butyl (piperidin-4-ylmethyl)carbamate (1.01 g, 4.70 mmol) was dissolved in dichloromethane (5.00 mL) and added to the isocyanate solution via syringe. The reaction mixture was stirred for 17 hours during which time a precipitate formed. The solid was filtered, washed with hexanes and dried in vacuo to yield the pure product (1.64 g, 88%). ¹H NMR (500 MHz, dmso-d₆) δ 8.84 (s, 1H), 7.67 (d, J=8.6 Hz, 2H), 7.56 (d, J=8.7 Hz, 2H), 6.91 (t, J=6.0 Hz, 1H), 4.10 (d, J=13.1 Hz, 2H), 2.83 (t, J=6.3 Hz, 2H), 2.75 (td, J=13.0, 2.5 Hz, 2H), 1.62 (d, J=13.2 Hz, 2H), 1.56 (tt, J=7.4, 4.1 Hz, 1H), 1.37 (s, 9H), 1.02 (qd, J=12.4, 4.0 Hz, 2H). ¹⁹F NMR (470 MHz, dmso-d₆) δ −59.97. HRMS ESI (+) calc'd for [M+Na]=424.1824, observed=424.1832.

### Example 75: MSU-45157/MBG XI-109. tert-Butyl N-methyl-N-(1-{[4-(trifluoromethyl)phenyl]carbamoyl}piperidin-4-yl)carbamate

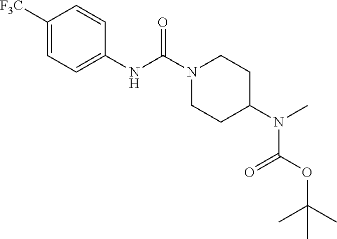 [SMILES: CN(C(=O)OC(C)(C)C)C1CCN(C(=O)Nc2ccc(C(F)(F)F)cc2)CC1]

Using general method A, 4-trifluoromethylphenyl isocyanate (0.823 g, 4.40 mmol) was dissolved in dichloromethane (5.00 mL) in a sealed vessel under an argon atmosphere. Tert-Butyl methyl(piperidin-4-yl)carbamate (0.996 g, 4.60 mmol) was dissolved in dichloromethane (5.00 mL) and added to the isocyanate solution via syringe. The reaction was stirred for 20 hours and concentrated in vacuo. The resulting solid was filtered, washed with hexanes and dried in vacuo to yield the pure product as a white solid (1.48 g, 84%). ¹H NMR (500 MHz, dmso-d₆) δ 8.90 (s, 1H), 7.70-7.63 (m, 2H), 7.56 (d, J=8.6 Hz, 2H), 4.21 (d, J=13.3 Hz, 2H), 4.10-3.69 (m, 1H), 2.87-2.75 (m, 2H), 2.65 (s, 3H), 1.56 (s, 4H), 1.39 (s, 9H). ¹⁹F NMR (470 MHz, dmso-d₆) δ −60.00. HRMS ESI (+) calc'd for [M+Na]=424.1824, observed=424.1834.

### Example 76: MSU-45012/MBG-XI-41. (3a,6a)-5-propanoyl-N-[4-(trifluoromethoxy)phenyl]-octahydropyrrolo[3,4-c]pyrrole-2-carboxamide

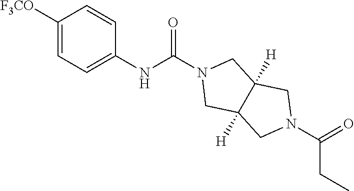 [SMILES: CCC(=O)N1C[C@@H]2CN(C(=O)Nc3ccc(OC(F)(F)F)cc3)C[C@@H]2C1]

tert-Butyl (3a,6a)-5-{[4-(trifluoromethoxy)phenyl]carbamoyl}-octahydropyrrolo[3,4-c]pyrrole-2-carboxylate (0.527 g, 1.41 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (7.00 ml) and the reaction vessel was sealed and flushed with argon. Triethylamine (0.505 g, 5.00 mmol) was added by syringe, followed by propionic anhydride (0.391 g, 3.00 mmol). The reaction mixture was stirred for 17 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product as a white solid (0.235 g, 45%). ¹H NMR (500 MHz, dmso-d₆) δ 8.33 (s, 1H), 7.64-7.53 (m, 2H), 7.21 (d, J=8.7 Hz, 2H), 3.71-3.57 (m, 3H), 3.52 (dd, J=12.2, 7.5 Hz, 1H), 3.35-3.17 (m, 4H), 3.01-2.93 (m, 1H), 2.87 (tt, J=9.9, 5.3 Hz, 1H), 2.22 (qd, J=7.7, 5.0 Hz, 2H), 0.95 (t, J=7.4 Hz, 3H). ¹⁹F NMR (470 MHz, dmso-d6) 6-57.10. HRMS ESI (+) calc'd for [M+H]=372.1531, observed=372.1559.

### Example 77: MSU-45013/MBG-XI-42. 2-Propanoyl-N-[4-(trifluoromethoxy)phenyl]-2,7-diazaspiro[3.5]nonane-7-carboxamide

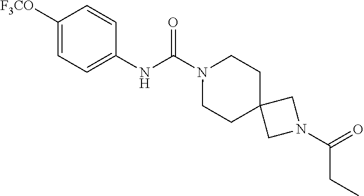 [SMILES: CCC(=O)N1CC2(CCN(C(=O)Nc3ccc(OC(F)(F)F)cc3)CC2)C1]

tert-Butyl 7-{[4-(trifluoromethoxy)phenyl]carbamoyl}-2,7-diazaspiro[3.5]nonane-2-carboxylate (0.329 g, 0.766 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (6.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 17 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo. This material was triturated from dichloromethane and hexanes, filtered and dried in vacuo to yield the pure product (0.192 g, 66%). ¹H NMR (500 MHz, dmso-d₆) δ 8.69 (s, 1H), 7.66-7.41 (m, 2H), 7.27-7.11 (m, 2H), 3.80 (s, 2H), 3.55 (s, 2H), 3.43-3.34 (m, 4H), 2.03 (q, J=7.5 Hz, 2H), 1.70-1.60 (m, 4H), 0.94 (t, J=7.5 Hz, 3H). ¹⁹F NMR (470 MHz, dmso-d₆) δ −57.11. HRMS ESI (+) calc'd for [M+H]=386.1687, observed=386.1716.

### Example 78: MSU-45014/MBG-11-43. 7-Propanoyl-N-[4-(trifluoromethoxy)phenyl]-2,7-diazaspiro[4.4]nonane-2-carboxamide

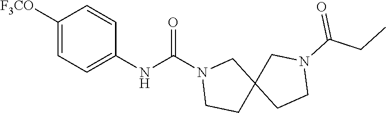 [SMILES: CCC(=O)N1CCC2(CCN(C(=O)Nc3ccc(OC(F)(F)F)cc3)C2)C1]

tert-Butyl 7-{[4-(trifluoromethoxy)phenyl]carbamoyl}-2,7-diazaspiro[4.4]nonane-2-carboxylate (0.320 g, 0.765 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 22 hours. The reaction was concentrated in vacuo and dried to a constant weight. Dichloromethane was added (6.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 17 hours, at which time it was concentrated in vacuo. The crude material was purified by by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo. This material was triturated from dichloromethane and hexanes, filtered and dried in vacuo to yield the pure product (0.133 g, 45%). ¹H NMR (500 MHz, dmso-d₆) δ 8.34 (d, J=2.0 Hz, 1H), 7.68-7.45 (m, 2H), 7.22 (d, J=8.7 Hz, 2H), 3.56-3.43 (m, 3H), 3.41-3.29 (m, 5H), 2.23 (dq, J=9.3, 7.4 Hz, 2H), 1.98-1.75 (m, 4H), 0.97 (td, J=7.4, 5.1 Hz, 3H). ¹⁹F NMR (470 MHz, dmso-d₆) δ −57.09. HRMS ESI (+) calc'd for [M+Na]=408.1511, observed=408.1539.

### Example 79: MSU-45015/MBG-XI-44. (3a,6a)-5-propanoyl-N-[4-(trifluoromethyl)phenyl]-octahydropyrrolo[3,4-c]pyrrole-2-carboxamide

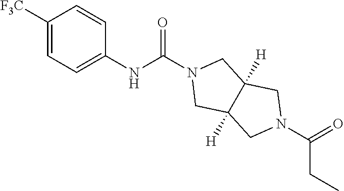 [SMILES: CCC(=O)N1C[C@@H]2CN(C(=O)Nc3ccc(C(F)(F)F)cc3)C[C@@H]2C1]

tert-Butyl (3a,6a)-5-{[4-(trifluoromethyl)phenyl]carbamoyl}-octahydropyrrolo[3,4-c]pyrrole-2-carboxylate. (0.382 g, 1.07 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (6.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo. This material was triturated from dichloromethane and hexanes, filtered and dried in vacuo to yield the pure product (0.353 g, 93%). ¹H NMR (500 MHz, dmso-d₆) δ 8.53 (s, 1H), 7.72 (d, J=8.5 Hz, 2H), 7.59-7.52 (m, 2H), 3.69-3.59 (m, 3H), 3.52 (dd, J=12.2, 7.5 Hz, 1H), 3.32-3.24 (m, 3H), 3.21 (dd, J=12.2, 4.7 Hz, 1H), 2.96 (h, J=6.7 Hz, 1H), 2.93-2.79 (m, 1H), 2.22 (qd, J=7.4, 4.7 Hz, 2H), 0.95 (t, J=7.4 Hz, 3H). ¹⁹F NMR (470 MHz, dmso-d₆) δ −59.99. HRMS ESI (+) calc'd for [M+H]=356.1582, observed=356.1616.

### Example 80: MSU-45088/MBG-XI-59. 2-Propanoyl-N-[4-(trifluoromethyl)phenyl]-2,7-diazaspiro[3.5]nonane-7-carboxamide. ‘ ’

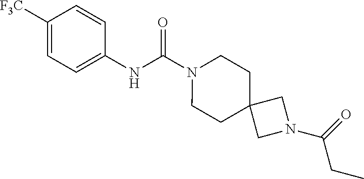 [SMILES: CCC(=O)N1CC2(CCN(C(=O)Nc3ccc(C(F)(F)F)cc3)CC2)C1]

tert-Butyl 7-{[4-(trifluoromethyl)phenyl]carbamoyl}-2,7-diazaspiro[3.5]nonane-2-carboxylate (0.512 g, 1.38 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (8.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 17 hours, at which time it was concentrated in vacuo. The crude material was purified by by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product (0.264 g, 52%). ¹H NMR (500 MHz, dmso-d₆) δ 8.90 (s, 1H), 7.67 (d, J=8.6 Hz, 2H), 7.56 (d, J=8.7 Hz, 2H), 3.82 (s, 2H), 3.56 (s, 2H), 3.41 (dt, J=6.8, 3.6 Hz, 4H), 2.04 (q, J=7.5 Hz, 2H), 1.72-1.62 (m, 4H), 0.95 (t, J=7.5 Hz, 3H). ¹⁹F NMR (470 MHz, dmso-d₆) δ −59.99. HRMS ESI (+) calc'd for [M+H]=370.1738, found=370.1765.

### Example 81: MSU-45089/MBG-XI-60. 7-propanoyl-N-[4-(trifluoromethyl)phenyl]-2,7-diazaspiro[4.4]nonane-2-carboxamide

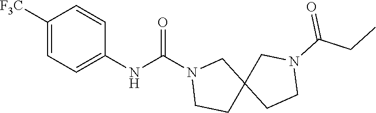 [SMILES: CCC(=O)N1CCC2(CCN(C(=O)Nc3ccc(C(F)(F)F)cc3)C2)C1]

tert-Butyl 7-{[4-(trifluoromethyl)phenyl]carbamoyl}-2,7-diazaspiro[4.4]nonane-2-carboxylate (0.584 g, 1.58 mmol) was dissolved in dichloromethane (8.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (8.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product (0.348 g, 60%). ¹H NMR (500 MHz, dmso-d₆) δ 8.53 (s, 1H), 7.74 (dd, J=8.7, 3.9 Hz, 2H), 7.57 (d, J=8.5 Hz, 2H), 3.56-3.45 (m, 3H), 3.45-3.35 (m, 4H), 3.32-3.21 (m, 1H), 2.23 (dq, J=9.6, 7.5 Hz, 2H), 1.96-1.78 (m, 4H), 0.97 (td, J=7.4, 5.3 Hz, 3H). ¹⁹F NMR (470 MHz, dmso-d₆) δ −59.97. HRMS ESI (+) calc'd for [M+H]=370.1738, observed=370.1769.

### Example 82: MSU-45107/MBG-XI-91. 4-Propanamido-N-[4-(trifluoromethyl)phenyl]piperidine-1-carboxamide

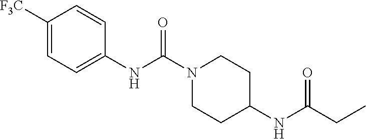 [SMILES: CCC(=O)NC1CCN(C(=O)Nc2ccc(C(F)(F)F)cc2)CC1]

tert-Butyl N-(1-{[4 (trifluoromethyl)phenyl]carbamoyl}piperidin-4-yl)carbamate (0.584 g, 1.50 mmol) was dissolved in dichloromethane (8.00 mL) The reaction vessel was sealed and flushed with argon. Trifluoroacetic acid (2.02 g, 17.7 mmol) was added via syringe and the reaction mixture was stirred for 20 hours. The reaction was concentrated in vacuo and dried to a constant weight. Dichloromethane was added (4.00 ml) and the reaction vessel was sealed and flushed with argon. Triethylamine (0.505 g, 5.00 mmol) was added by syringe, followed by propionic anhydride (0.391 g, 3.00 mmol). The reaction mixture was stirred for 20 hours, during which time a solid precipitated. The solid was filtered, washed with a solution of 10% dichloromethane in hexanes and dried in vacuo to yield the pure product (0.262 g, 51%). ¹H NMR (500 MHz, dmso-d₆) δ 8.90 (s, 1H), 7.73 (d, J=7.8 Hz, 1H), 7.67 (d, J=8.6 Hz, 2H), 7.57 (d, J=8.6 Hz, 2H), 4.06-3.97 (m, 2H), 3.76 (td, J=9.1, 5.1 Hz, 1H), 2.94 (ddd, J=13.8, 11.6, 2.7 Hz, 2H), 2.05 (q, J=7.6 Hz, 2H), 1.74 (dd, J=12.9, 3.8 Hz, 2H), 1.35-1.22 (m, 2H), 0.98 (t, J=7.6 Hz, 3H). ¹⁹F NMR (470 MHz, dmso-d₆) δ −59.98. HRMS ESI (+) Calc'd for [M+Na]=366.1406, observed=366.1426.

### Example 83: MSU-45108/MBG-XI-92. 3-Propanamido-N-[4-(trifluoromethyl)phenyl]piperidine-1-carboxamide

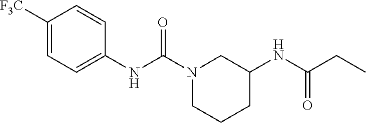 [SMILES: CCC(=O)NC1CCCN(C(=O)Nc2ccc(C(F)(F)F)cc2)C1]

tert-Butyl N-(1-{[4-(trifluoromethyl)phenyl]carbamoyl}piperidin-3-yl)carbamate (0.511 g, 1.31 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (8.00 ml) and the reaction vessel was sealed and flushed with argon. Triethylamine (0.606 g, 6.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product (0.339 g, 71%). ¹H NMR (500 MHz, dmso-d₆) δ 8.86 (s, 1H), 7.76 (d, J=7.7 Hz, 1H), 7.65 (d, J=8.5 Hz, 2H), 7.55 (d, J=8.7 Hz, 2H), 3.90 (dd, J=12.9, 4.0 Hz, 1H), 3.80 (dd, J=11.0, 6.5 Hz, 1H), 3.65-3.55 (m, 1H), 2.95 (ddd, J=13.2, 10.2, 3.0 Hz, 1H), 2.79 (dd, J=12.8, 9.3 Hz, 1H), 2.04 (qd, J=7.5, 2.0 Hz, 2H), 1.85-1.76 (m, 1H), 1.75-1.67 (m, 1H), 1.47-1.31 (m, 2H), 0.94 (t, J=7.6 Hz, 3H). ¹⁹F NMR (470 MHz, dmso-d₆) δ −59.99. HRMS ESI (+) Calc'd for [M+Na]=366.1406, observed=366.1425.

### Example 84: MSU-45152/MBG XI-101. 3-(Propanamidomethyl)-N-[4-(trifluoromethyl)phenyl]piperidine-1-carboxamide

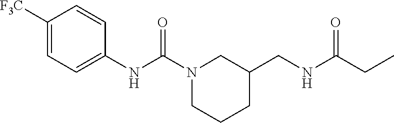 [SMILES: CCC(=O)NCC1CCCN(C(=O)Nc2ccc(C(F)(F)F)cc2)C1]

tert-Butyl N-[(1-{[4-(trifluoromethyl)phenyl]carbamoyl}piperidin-3-yl)methyl]carbamate (0.517 g, 1.45 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (5.00 ml) and the reaction vessel was sealed and flushed with argon. Triethylamine (0.808 g, 8.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product (0.269 g, 52%). ¹H NMR (500 MHz, dmso-d₆) δ 8.86 (s, 1H), 7.80 (t, J=5.8 Hz, 1H), 7.65 (d, J=8.7 Hz, 2H), 7.55 (d, J=8.6 Hz, 2H), 4.02-3.93 (m, 1H), 3.90 (d, J=13.3 Hz, 1H), 2.99 (dt, J=13.1, 6.5 Hz, 1H), 2.96-2.81 (m, 2H), 2.55 (dd, J=13.1, 10.2 Hz, 1H), 2.07 (q, J=7.6 Hz, 2H), 1.77-1.69 (m, 1H), 1.69-1.60 (m, 1H), 1.55 (tt, J=6.8, 3.6 Hz, 1H), 1.35 (q, J=11.9 Hz, 1H), 1.19-1.07 (m, 1H), 0.98 (t, J=7.6 Hz, 3H). ¹⁹F NMR (470 MHz, dmso-d₆) δ −59.97. HRMS ESI (+) calc'd for [M+H]=358.1738, observed=358.1772.

### Example 85: MSU-45154/MBG-XI-104. 7-Propanoyl-N-[4-(trifluoromethyl)phenyl]-2,7-diazaspiro[4.5]decane-2-carboxamide

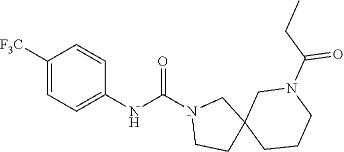 [SMILES: CCC(=O)N1CCCC2(CCN(C(=O)Nc3ccc(C(F)(F)F)cc3)C2)C1]

tert-Butyl 2-{[4-(trifluoromethyl)phenyl]carbamoyl}-2,7-diazaspiro[4.5]decane-7-carboxylate (0.517 g, 1.45 mmol) was dissolved in dichloromethane (5.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (5.00 ml) and the reaction vessel was sealed and flushed with argon. Triethylamine (0.808 g, 8.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo. The pure product was obtained by dissolving the resultant solid in dichloromethane and triturating with ethyl acetate (0.026 g, 5%). ¹H NMR (500 MHz, CD₃OD) δ 7.63 (d, J=8.5 Hz, 2H), 7.53 (dd, J=8.7, 4.9 Hz, 2H), 3.65-3.57 (m, 2H), 3.51 (td, J=9.1, 3.7 Hz, 3H), 3.40 (d, J=10.6 Hz, 1H), 3.18 (dd, J=10.6, 3.4 Hz, 1H), 2.49-2.31 (m, 2H), 1.96-1.48 (m, 7H), 1.16-1.04 (m, 3H). ¹⁹F NMR (470 MHz, CD₃OD) δ −63.39 (d, J=9.7 Hz). HRMS ESI (+) calc'd for [M+H]=384.1895, observed=384.1913.

### Example 86: MSU-45155/MBG-XI-106. tert-Butyl 6-{[4-(trifluoromethyl)phenyl]carbamoyl}-2,6-diazaspiro[3.4]octane-2-carboxylate

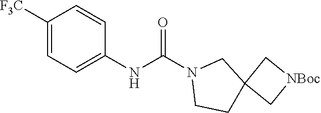 [SMILES: CC(C)(C)OC(=O)N1CC2(CCN(C(=O)Nc3ccc(C(F)(F)F)cc3)C2)C1]

Using general method A, 4-trifluoromethylphenyl isocyanate (0.299 g, 1.60 mmol) was dissolved in dichloromethane (4.00 mL) in a sealed vessel under an argon atmosphere. tert-Butyl-2,6-diazaspiro[3,4]octane-2-carboxylate (0.370, 1.70 mmol) was dissolved in dichloromethane (1.0 mL) and added to the isocyanate solution by syringe. The reaction mixture was stirred for 22 hours, during which time a white solid precipitated. This material was filtered, washed with hexanes and dried in vacuo to yield the pure product (0.491 g, 77%). ¹H NMR (500 MHz, dmso-d₆) δ 8.53 (s, 1H), 7.72 (d, J=8.6 Hz, 2H), 7.56 (d, J=8.6 Hz, 2H), 3.77 (d, J=17.0 Hz, 4H), 3.54 (s, 2H), 3.41 (t, J=6.9 Hz, 2H), 2.05 (t, J=6.9 Hz, 2H), 1.36 (s, 9H). ¹⁹F NMR (470 MHz, dmso-d₆) δ −59.99. HRMS ESI (+) calc'd for [M+Na]=422.1668, observed=422.1676.

### Example 87: MSU-45020/MBG-XI-110. 2-propanoyl-N-[4-(trifluoromethyl)phenyl]-2,6-diazaspiro[3.4]octane-6-carboxamide

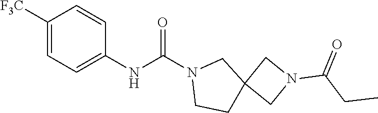 [SMILES: CCC(=O)N1CC2(CCN(C(=O)Nc3ccc(C(F)(F)F)cc3)C2)C1]

tert-Butyl 6-{[4-(trifluoromethyl)phenyl]carbamoyl}-2,6-diazaspiro[3.4]octane-2-carboxylate (0.410 g, 1.15 mmol) was dissolved in dichloromethane (3.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (5.00 ml) and the reaction vessel was sealed and flushed with argon. Triethylamine (0.808 g, 8.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to yield the pure product (0.206 g, 50%). ¹H NMR (500 MHz, CDCl₃) δ 7.53 (s, 4H), 6.40 (s, 1H), 4.15-4.01 (m, 2H), 3.96 (q, J=9.9 Hz, 2H), 3.67 (q, J=10.3 Hz, 2H), 3.63-3.47 (m, 2H), 2.21 (t, J=6.9 Hz, 2H), 2.12 (q, J=7.5 Hz, 2H), 1.13 (t, J=7.5 Hz, 3H). ¹⁹F NMR (470 MHz, CDCl₃) δ −61.93. HRMS ESI (+) calc'd for [M+H]=356.1582, observed=356.1598.

### Example 88: MSU-45158/MBG XI-111. 4-(N-methylpropanamido)-N-[4-(trifluoromethyl)phenyl]piperidine-1-carboxamide

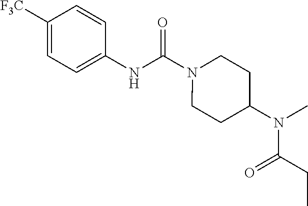 [SMILES: CCC(=O)N(C)C1CCN(C(=O)Nc2ccc(C(F)(F)F)cc2)CC1]

tert-Butyl N-methyl-N-(1-{[4-(trifluoromethyl)phenyl]carbamoyl}piperidin-4-yl)carbamate (0.584 g, 1.63 mmol) was dissolved in dichloromethane (3.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (5.00 ml) and the reaction vessel was sealed and flushed with argon. Triethylamine (0.808 g, 8.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo. The pure product was obtained by recrystallizing the solid from dichloromethane and hexanes (0.160 g, 27%). ¹H NMR (500 MHz, CDCl₃) δ 7.54 (d, J=8.6 Hz, 2H), 7.48 (d, J=8.6 Hz, 2H), 6.56 (s, 1H), 4.79-4.69 (m, 1H), 4.27-4.12 (m, 2H), 3.05-2.92 (m, 2H), 2.84 (s, 3H), 2.44-2.29 (m, 2H), 1.92-1.60 (m, 4H), 1.17 (dt, J=14.8, 7.4 Hz, 3H). ¹⁹F NMR (470 MHz, CDCl₃) δ −61.93, −61.96. HRMS ESI (+) calc'd for [M+H]=358.1753, observed=358.1753.

### Example 89: MSU-45159/MBG-XI-94. 3-Methyl-3-(propanamidomethyl)-N-[4-(trifluoromethyl)phenyl]pyrrolidine-1-carboxamide

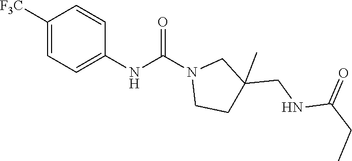 [SMILES: CCC(=O)NCC1(C)CCN(C(=O)Nc2ccc(C(F)(F)F)cc2)C1]

tert-Butyl N-[(3-methyl-1-{[4-(trifluoromethyl)phenyl]carbamoyl}pyrrolidin-3-yl)methyl]carbamate. (0.280 g, 0.784 mmol) was dissolved in dichloromethane (3.00 mL) The reaction vessel was sealed and flushed with argon. Trifluoroacetic acid (2.70 g, 23.7 mmol) was added via syringe and the reaction mixture was stirred for 20 hours. The reaction was concentrated in vacuo and dried to a constant weight. Dichloromethane was added (5.00 ml) and the reaction vessel was sealed and flushed with argon. Triethylamine (0.505 g, 5.00 mmol) was added by syringe, followed by propionic anhydride (0.391 g, 3.00 mmol). The reaction mixture was stirred for 18 hours, at which time it was concentrated in vacuo. The crude material was purified by silica gel chromatography (0 to 10% methanol in dichloromethane). Fractions containing product were combined, concentrated, and purified by reverse-phase medium pressure liquid chromatography (0 to 100% methanol in 25 mM aqueous ammonium formate). The fractions containing product were combined, concentrated, partitioned between ethyl acetate and saturated aqueous sodium bicarbonate, washed with brine, dried over sodium sulfate, and concentrated in vacuo to give the pure product (0.210 g, 75%). ¹H NMR (500 MHz, CD₃OD) δ 7.69-7.60 (m, 2H), 7.60-7.48 (m, 2H), 3.67-3.51 (m, 2H), 3.38 (d, J=10.4 Hz, 1H), 3.28 (s, 1H), 3.20 (d, J=10.5 Hz, 2H), 2.25 (q, J=7.6 Hz, 2H), 1.91 (dt, J=13.8, 7.2 Hz, 1H), 1.73 (dt, J=13.4, 7.2 Hz, 1H), 1.19-1.07 (m, 6H). ¹⁹F NMR (470 MHz, CD₃OD) δ −63.36 (d, J=3.2 Hz). ESI (+), calculated M+H=358.1738, observed=358.1759.

### Example 90: MSU-45153/MBG-XI-103. N-[4-(trifluoromethyl)phenyl]-2,7-diazaspiro[4.5]decane-2-carboxamide trifluoroacetic acid

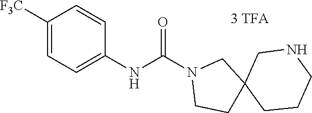 [SMILES: O=C(Nc1ccc(C(F)(F)F)cc1)N1CCC2(CCCNC2)C1]

tert-Butyl 2-{[4-(trifluoromethyl)phenyl]carbamoyl}-2,7-diazaspiro[4.5]decane-7-carboxylate (0.360 g, 0.842 mmol) was dissolved in dichloromethane (5.00 mL) and the reaction vessel was sealed and flushed with argon. Trifluoroacetic acid (1.35 g, 11.8 mmol) was added via syringe and the reaction mixture was stirred for 19 hours. The reaction was concentrated in vacuo and dried to constant weight (0.563 g, 100% yield). ¹H NMR (500 MHz, CD₃OD) δ 7.64 (d, J=8.5 Hz, 2H), 7.56 (d, J=8.6 Hz, 2H), 3.68-3.60 (m, 2H), 3.53-3.39 (m, 2H), 3.24-3.12 (m, 4H), 2.03 (qd, J=13.1, 6.4 Hz, 2H), 1.87 (dt, J=11.9, 7.2 Hz, 2H), 1.82-1.70 (m, 2H). ¹⁹F NMR (470 MHz, CD₃OD) δ −63.42 (3F), −77.47 (9F). HRMS ESI (+) calc'd for [M+H]=328.1632, observed=328.1653.

### Example 91: MSU-43895/ycl-1-10a

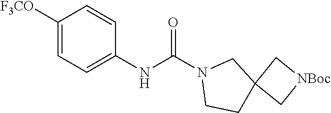 [SMILES: CC(C)(C)OC(=O)N1CC2(CCN(C(=O)Nc3ccc(OC(F)(F)F)cc3)C2)C1]

Using general method A, 4-trifluoromethylphenyl isocyanate (0.561 g, 3.17 mmol) was dissolved in dichloromethane (1.00 mL) in a sealed vessel under an argon atmosphere. Tert-butyl 2,6-diazaspiro[3.4]octane-2-carboxylate (0.673 g, 3 mmol) was dissolved in dichloromethane (10.0 mL) and added to the isocyanate solution by syringe. The reaction was stirred for 18 hours, concentrated in vacuo and purified by silica gel chromatography (0 to 10% methanol in dichloromethane) to yield the pure product (0.718 g, 57%). ¹H NMR (500 MHz, cdc13) δ 7.59-7.45 (m, 4H), 6.28 (s, 1H), 3.93 (d, J=8.7, 2H), 3.87 (d, J=8.7, 2H), 3.64 (s, 2H), 3.54 (t, J=6.8, 2H), 2.20 (t, J=6.8, 2H), 1.45 (s, 9H). ¹⁹F NMR (470 MHz, Chloroform-d) 6-61.94. HRMS ESI (+) calc'd for [M+Na]=422.1662, observed=422.1709.

### Example 92: MSU-43895/ycl-1-12c

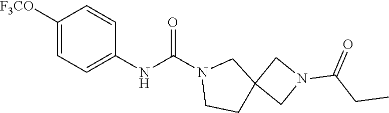 [SMILES: CCC(=O)N1CC2(CCN(C(=O)Nc3ccc(OC(F)(F)F)cc3)C2)C1]

MSU-43895/ycl-1-10a (0.2 g, 0.5 mmol) was dissolved in dichloromethane (2.50 mL) The reaction vessel was sealed and flushed with argon. Trifluoroacetic acid (0.77 mL, 10.0 mmol) was added via syringe and the reaction mixture was stirred for 23 hours. The reaction was concentrated in vacuo and dried to a constant weight. Dichloromethane was added (1.30 mL) and the reaction vessel was sealed and flushed with argon. Triethylamine (0.18 mL, 1.3 mmol) was added by syringe, followed by propionic anhydride (0.08 mL, 0.65 mmol). The reaction mixture was stirred for 16.5 hours, at which time it was concentrated in vacuo. The crude material was purified by silica gel chromatography (0 to 10% methanol in dichloromethane). The fractions containing product were combined, concentrated, in vacuo to yield the pure product (0.045 g, 25%). 1H NMR (500 MHz, Chloroform-d) δ 7.58-7.47 (m, 4H), 6.32 (s, 1H), 4.12-3.94 (m, 2H), 3.68 (s, 2H), 3.57 (t, J=6.9, 2H), 2.23 (t, J=6.9, 2H), 2.13 (q, J=7.5, 2H), 1.14 (t, J=7.5, 3H). 19F NMR (470 MHz, Chloroform-d) 6-61.95. HRMS ESI (+) calc'd for [M+H]=356.1580, observed=356.1615.

### Example 93: MSU-45170/KSL-0057. 1-(7-propionyl-7-azaspiro[3.5]nonan-2-yl)-3-(4-(trifluoromethoxy)phenyl)urea

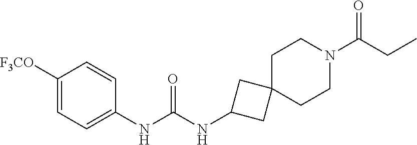 [SMILES: CCC(=O)N1CCC2(CC1)CC(NC(=O)Nc1ccc(OC(F)(F)F)cc1)C2]

The title compound was prepared in 45% (32 mg) yield using 1-(7-azaspiro[3.5]nonan-2-yl)-3-(4-(trifluoromethoxy)phenyl)urea (50 mg, μmol) via general method G. ¹H NMR (500 MHz, DMSO-d6) δ 8.58 (s, 1H), 7.47 (d, J=7.5 Hz, 2H), 7.21 (d, J=7.5 Hz, 2H), 6.51 (d, J=10 Hz, 1H), 4.18-4.06 (m, 1H), 3.37-3.43 (m, 1H), 3.24-3.30 (m, 1H), 2.28 (q, J=0.7.5 Hz, 2H), 2.15-2.23 (m, 3H), 1.59-1.68 (m, 2H), 1.51-1.57 (m, 1H), 1.44-1.51 (m, 2H), 1.38-1.44 (m, 1H), 0.96 (t, J=7.5 Hz, 3H). Purity: >95%

### Example 94: MSU-45186/KSL-0063. 1-((1R,4R)-2-propionyl-2-azabicyclo[2.1.1]hexan-5-yl)-3-(4-(trifluoromethoxy)phenyl)urea

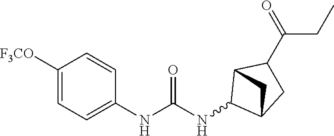 [SMILES: CCC(=O)C1C[C@H]2C[C@@H]1C2NC(=O)Nc1ccc(OC(F)(F)F)cc1]

The title compound was prepared in 21.1% (30 mg) yield using 1-((1R,4R)-2-azabicyclo[2.1.1]hexan-5-yl)-3-(4-(trifluoromethoxy)phenyl)urea (35 mg, μmol) via general method G. ¹H NMR (500 MHz, DMSO-d6), diastereoisomers A: δ 8.72 (s, 1H), 7.44 (d, J=5 Hz, 2H), 7.22 (d, J=5 Hz, 2H), 6.35 (d, J=10 Hz, 1H), 4.38 (d, J=10 Hz, 1H), 3.92 (d, J=5 Hz, 1H), 3.24-3.22 (m, 1H), 3.15 (d, J=10 Hz, 1H), 2.77-7.75 (m, 1H), 2.21-2.26 (m, 2H), 1.67 (d, J=5 Hz, 1H), 1.17 (d, J=10 Hz, 1H), 0.99 (t, J=7.5 Hz, 3H).

Diastereoisomers B: δ 8.65 (s, 1H), 7.45 (d, J=10 Hz, 2H), 7.22 (d, J=5 Hz, 2H), 6.27 (d, J=10 Hz, 1H), 4.50 (d, J=10 Hz, 1H), 3.85 (d, J=10 Hz, 1H), 3.39 (d, J=5 Hz, 1H), 3.24-3.22 (m, 1H), 2.83-2.82 (m, 1H), 2.25-2.33 (m, 2H), 2.24 (dq, J=8.33, J=2.5 Hz, 2H), 1.59 (d, J=10 Hz, 1H), 1.10 (d, J=5 Hz, 1H), 1.04 (t, J=7.5 Hz, 2H).

Purity: ≥95% Melting Point (° C.): HRMS (found (ESI(+), [M−H]⁺)

### Example 95: MSU-45187/KSL-0064. 1-((1R,4R)-2-propionyl-2-azabicyclo[2.2.1]heptan-7-yl)-3-(4-(trifluoromethoxy)phenyl)urea

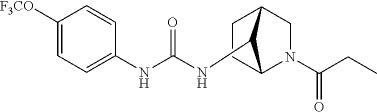 [SMILES: CCC(=O)N1C[C@H]2CC[C@@H]1C2NC(=O)Nc1ccc(OC(F)(F)F)cc1]

The title compound was prepared in 16% (12.7 mg) yield using 1-((1R,4R)-2-azabicyclo[2.2.1]heptan-7-yl)-3-(4-(trifluoromethoxy)phenyl)urea (65.9 mg, 209 μmol) via general method G. ¹H NMR (500 MHz, DMSO-d6), diastereoisomers mixture: δ 8.61 (dd, J=10, 5 Hz, 1H), 7.48 (d, J=5 Hz, 2H), 7.23 (d, J=10 Hz, 2H), 6.42 (dd, J=5, 10 Hz, 1H), 4.26 (s, 1H), 4.10 (s, 1H), 3.79 (d, J=5 Hz, 1H), 3.74 (d, J=5 Hz, 1H), 3.47-3.52 (m, 1H), 3.28 (d, J=10 Hz, 1H), 3.10 (d, J=10 Hz, 1H), 2.98 (d, J=10 Hz, 1H), 2.43 (br, 1H), 2.38 (br, 1H), 2.24-2.34 (m, 2H), 2.06-2.18 (m, 4H), 1.80-1.83 (m, 2H), 1.70-1.77 (m, 2H), 1.53-1.61 (m, 2H) 1.40-1.6 (m, 4H), 0.98 (t, J=10 Hz, 3H), 0.95 (t, J=10 Hz, 3H).

Purity: ≥95% Melting Point (° C.): HRMS (found (ESI(+), [M−H]⁺)

### Example 96: MSU-45188/KSL-0060. 1-(4-(difluoromethoxy)phenyl)-3-(1-propionylpiperidin-4-yl)urea

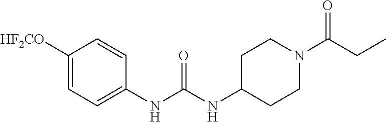

The title compound was prepared in 71% (65 mg) yield using 1-(difluoromethoxy)-4-isocyanatobenzene (50 mg, 270 μmol) and 1-(4-aminopiperidin-1-yl)propan-1-one (42 mg, 270 μmol) via general method C. ¹H NMR (500 MHz, DMSO-d6): δ 8.58 (s, 1H), 7.41 (d, J=5 Hz, 2H), 7.08 (t, J=75 Hz, 2H), 7.04 (d, J=5 Hz, 2H), 6.34 (d, J=10 Hz, 1H), 4.18 (d, J=10 Hz, 1H), 3.76 (d, J=10 Hz, 1H), 3.64-3.72 (m, 1H), 3.11 (t, J=10 Hz, 1H), 2.78 (t, J=10 Hz, 1H), 2.32 (q, J=10 Hz, 2H), 1.75-1.87 (m, 2H), 1.25-1.35 (m, 1H), 1.15-1.25 (m, 1H), 0.98 (t, J=7.5 Hz, 3H).

### Example 97: MSU-45189/KSL-0058. 1-propionylpiperidin-4-yl (4-(trifluoromethoxy)phenyl)carbamate

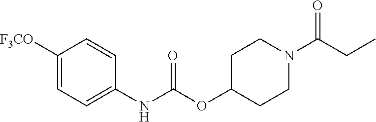 [SMILES: CCC(=O)N1CCC(OC(=O)Nc2ccc(OC(F)(F)F)cc2)CC1]

The title compound was prepared in 22% (36.5 mg) yield using 1-(4-hydroxypiperidin-1-yl)propan-1-one (73.7 mg, 469 μmol) via general method C. ¹H NMR (500 MHz, DMSO-d6): δ 9.88 (s, 1H), 7.55 (d, J=10 Hz, 2H), 7.29 (d, J=10 Hz, 2H), 4.82-4.90 (m, 1H), 3.90-3.97 (m, 1H), 3.68-3.72 (m, 1H), 3.26-3.33 (m, 1H), 3.10-3.16 (m, 1H), 3.33 (q, J=7.5 Hz, 2H), 1.85-1.97 (m, 2H), 1.42-1.60 (m, 2H), 0.98 (t, J=7.5 Hz, 3H).

### Example 98: MSU-45190/KSL-0065. 1-isobutyrylpiperidin-4-yl (4-(trifluoromethoxy)phenyl)carbamate

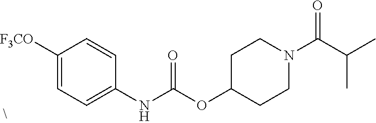 [SMILES: CC(C)C(=O)N1CCC(OC(=O)Nc2ccc(OC(F)(F)F)cc2)CC1]

The title compound was prepared in 39% (60 mg) yield using 1-(4-hydroxypiperidin-1-yl)-2-methylpropan-1-one (70.1 mg, 409 μmol) via general method C. ¹H NMR (500 MHz, DMSO-d6): δ 8.67 (s, 1H), 7.55 (d, J=10 Hz, 2H), 7.22 (d, J=10 Hz, 2H), 3.78-3.84 (m, 2H), 3.63-3.70 (m, 1H), 3.03-3.08 (m, 2H), 2.82-2.88 (m, 1H), 1.70-1.77 (m, 2H), 1.25-1.35 (m, 2H), 0.95-0.99 (m, 6H).

### Example 99: MSU-45191/KSL-0055. 1-(1-(3-fluoropropanoyl)piperidin-4-yl)-3-(4-(trifluoromethoxy)phenyl)urea

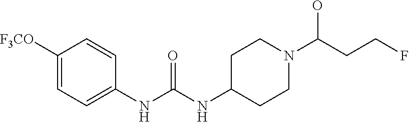 [SMILES: O=C(Nc1ccc(OC(F)(F)F)cc1)NC1CCN(C(O)CCF)CC1]

The title compound was prepared in 77% (96 mg) yield using 1-(piperidin-4-yl)-3-(4-(trifluoromethoxy)phenyl)urea (100 mg, 330 μmol) and 3-fluoropropanoic acid (36 mg, 396 μmol) via general method G. ¹H NMR (500 MHz, DMSO-d6): δ 8.64 (s, 1H), 7.48 (d, J=5 Hz, 2H), 7.22 (d, J=5 Hz, 2H), 6.33 (d, J=10 Hz, 1H), 4.66 (td, J=5 Hz, 45 Hz, 2H), 4.20 (d, J=15 Hz, 1H), 3.80 (d, J=15 Hz, 1H), 3.66-3.82 (m, 1H), 3.14 (t, J=10 Hz, 1H), 2.72-2.85 (m, 3H), 1.75-1.88 (m, 2H), 1.15-1.38 (m, 2H).

### Example 100: MSU45192/KSL-0064-Boc. tert-butyl (1R,4R)-7-(3-(4-(trifluoromethoxy)phenyl)ureido)-2-azabicyclo[2.2.1]heptane-2-carboxylate

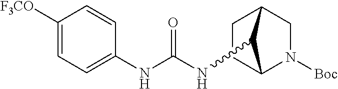 [SMILES: CC(C)(C)OC(=O)N1C[C@H]2CC[C@@H]1C2NC(=O)Nc1ccc(OC(F)(F)F)cc1]

The title compound was prepared in 70% yield (112.3 mg) using 4-(Trifluoromethoxy)phenyl isocyanate (77.5 mg, 379 μmol) and tert-butyl (1R,4R)-7-amino-2-azabicyclo[2.2.1]heptane-2-carboxylate (89.2 mg, 421 μmol) via general method C. The final product was purified by silica gel chromatography (EtOAc:Hex/1:1). ¹H NMR (500 MHz, DMSO-d6): δ 0.8.57 (s, 1H), 7.47 (d, J=10 Hz, 2H), 7.23 (d, J=10 Hz, 2H), 6.40 (t, J=10 Hz, 1H), 3.92-3.98 (m, 1H), 3.70-3.75 (m, 1H), 3.23-3.33 (m, 1H), 2.91-2.94 (m, 1H), 2.31-2.34 (br, 1H), 1.66-1.81 (m, 3H), 1.41-1.57 (m, 4H), 1.39 (s, 9H).

### Example 101: MSU45193/KSL-0063-Boc. tert-butyl (1R,4R)-5-(3-(4-(trifluoromethoxy)phenyl)ureido)-2-azabicyclo[2.1.1]hexane-2-carboxylate

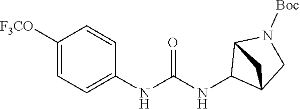 [SMILES: CC(C)(C)OC(=O)N1C[C@H]2C[C@@H]1C2NC(=O)Nc1ccc(OC(F)(F)F)cc1]

The title compound was prepared in 74% yield (120 mg) using 4-(Trifluoromethoxy)phenyl isocyanate (82.2 mg, 405 μmol) and tert-butyl (1R,4R)-5-amino-2-azabicyclo[2.1.1]hexane-2-carboxylate (88.3 mg, 446 μmol) via general method C. The final product was purified by silica gel chromatography (EtOAc:Hex/1:1). ¹H NMR (500 MHz, DMSO-d6): δ 0.8.57 (s, 1H), 7.47 (d, J=10 Hz, 2H), 7.23 (d, J=10 Hz, 2H), 6.40 (t, J=10 Hz, 1H), 3.92-3.98 (m, 1H), 3.70-3.75 (m, 1H), 3.23-3.33 (m, 1H), 2.91-2.94 (m, 1H), 2.31-2.34 (br, 1H), 1.66-1.81 (m, 3H), 1.41-1.57 (m, 4H), 1.39 (s, 9H).

### Example 102: MSU45194/KSL-0068-Boc. tert-butyl (2S,3S)-2-fluoro-3-(3-(4-(trifluoromethoxy)phenyl)ureido)pyrrolidine-1-carboxylate

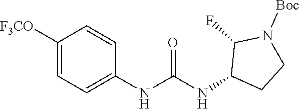 [SMILES: CC(C)(C)OC(=O)N1CC[C@H](NC(=O)Nc2ccc(OC(F)(F)F)cc2)[C@@H]1F]

The title compound was prepared in 32% yield (58 mg) using 4-(Trifluoromethoxy)phenyl isocyanate (90.4 mg, 445 μmol) and tert-butyl (2S,3S)-3-amino-2-fluoropyrrolidine-1-carboxylate (100 mg, 490 μmol) via general method C. The final product was purified by silica gel chromatography (EtOAc:Hex/1:1). ¹H NMR (500 MHz, DMSO-d6): δ 0.8.80 (s, 1H), 7.48 (d, J=7.5 Hz, 2H), 7.25 (d, J=7.5 Hz, 2H), 6.57-6.62 (m, 1H), 5.04-5.20 (m, 1H), 4.32-4.47 (m, 1H), 4.32-4.45 (m, 1H), 3.64-3.73 (m, 1H), 3.49-3.59 (m, 1H), 2.94-3.03 (m, 2H), 1.41 (s, 9H).

### Example 103: MSU-45195/KSL-0068. 1-((2S,3S)-2-fluoro-1-propionylpyrrolidin-3-yl)-3-(4-(trifluoromethoxy)phenyl)urea

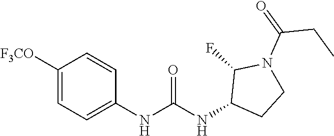 [SMILES: CCC(=O)N1CC[C@H](NC(=O)Nc2ccc(OC(F)(F)F)cc2)[C@@H]1F]

The title compound was prepared in 75% (19.1 mg) yield using tert-butyl (2S,38)-2-fluoro-3-(3-(4-(trifluoromethoxy)phenyl)ureido)pyrrolidine-1-carboxylate (21.5 mg, 70 μmol) via general method G. ¹H NMR (500 MHz, DMSO-d6): δ 8.81 (d, J=15 Hz, 1H), 7.49 (d, J=7.5 Hz, 2H), 7.25 (d, J=7.5 Hz, 2H), 6.63 (t, J=7.5 Hz, 1H), 5.05-5.30 (m, 1H), 4.30-4.55 (m, 1H), 3.61-3.91 (m, 2H), 3.47-3.61 (m, 1H), 2.15-2.35 (m, 2H), 0.96-1.01 (m, 3H).

### Example 104: MSU-45196/KSL-0067. (3S,4R)-3-fluoro-1-isobutyrylpiperidin-4-yl (4-(trifluoromethoxy)phenyl)carbamate

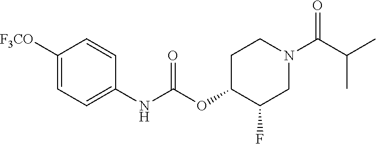 [SMILES: CC(C)C(=O)N1CC[C@@H](OC(=O)Nc2ccc(OC(F)(F)F)cc2)[C@@H](F)C1]

The title compound was prepared in 11.3% (67.4 mg) yield using 1-((3S,4R)-3-fluoro-4-hydroxypiperidin-1-yl)-2-methylpropan-1-one (287.5 mg, 1.52 mmol) via general method C. ¹H NMR (500 MHz, DMSO-d6): δ 10.04 (s, 1H), 7.57 (d, J=7.5 Hz, 2H), 7.30 (d, J=7.5 Hz, 2H), 4.85-5.04 (m, 2H), 4.37 (dt, J=165 Hz, 12.5 Hz, 1H), 4.15 (dd, J=215 HZ, 12.5 Hz, 1H), 2.75-3.55 (m, 3H), 1.60-1.94 (m, 2H), 0.99 (t, J=7.5 Hz, 6H).

### Example 105: MSU-45197/KSL-0066. (S)-1-(2-methylbutanoyl)piperidin-4-yl (4-(trifluoromethoxy)phenyl)carbamate

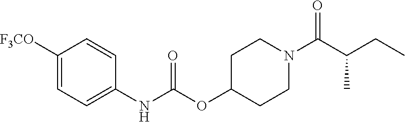 [SMILES: CC[C@H](C)C(=O)N1CCC(OC(=O)Nc2ccc(OC(F)(F)F)cc2)CC1]

The title compound was prepared in 21% (114 mg) yield using (S)-1-(4-hydroxypiperidin-1-yl)-2-methylbutan-1-one (264.6 mg, 1.43 mmol) via general method C. ¹H NMR (500 MHz, DMSO-d6): δ 9.89 (s, 1H), 7.56 (d, J=7.5 Hz, 2H), 7.30 (d, J=7.5 Hz, 2H), 4.85-5.04 (m, 1H), 3.95-4.05 (m, 1H), 3.76-3.86 (m, 1H), 3.10-3.21 (m, 1H), 2.70-2.76 (m, 1H), 1.84-2.06 (m, 2H), 1.40-1.60 (m, 2H), 1.21-1.34 (m, 1H), 0.95-1.00 (m, 3H), 0.81 (t, J=7.5 Hz, 3H)

### Example 106: Physical Properties and IC₅₀ Determination for Human sEH Inhibitors

IC₅₀s of hsEH inhibitors were determined by fluorescence according to Lee et al. Optimized inhibitors of soluble epoxide hydrolase improve in vitro target residence time and in vivo efficacy. J Med Chem. 2014; 57(16):7016-30; and Lee et al. Forster resonance energy transfer competitive displacement assay for human soluble epoxide hydrolase. Anal Biochem. 2013; 434(2):259-68, which are incorporated herein by reference in their entirety.

Concentration of the inhibitor that blocks 50% of the enzyme activity was determined based on regression of at least five datum points with a minimum of two points in the linear region of the curve on either side of the IC₅₀.

### FRET-Displacement Assay Procedure

The FRET assay was carried out as described previously in Lee et al. Optimized inhibitors of soluble epoxide hydrolase improve in vitro target residence time and in vivo efficacy. J Med Chem. 2014; 57(16):7016-30 and Lee et al. Forster resonance energy transfer competitive displacement assay for human soluble epoxide hydrolase. Anal Biochem. 2013; 434(2):259-68. In order to prevent leaching of fluorescence impurities from the plastic tube and non-specific binding to sEH inhibitors, the inhibitor stock solution (10 mM, DMSO) was stored in glass vials. In addition, the recombinant affinity purified sEH was diluted to the desired concentration (20 nM) with sodium phosphate buffer (PB) (100 mM sodium phosphate, pH 7.4, 0.01% gelatin) to avoid loss of protein by non-specific binding to the cuvette surface. All buffer used in this assay was filtered with a sterilized filtration unit (Millipore® Durapore PVDF Membrane, pore size: 0.22 um).

### Measurement in 96-Well Plates

All the measurement for FRET-based displacement assay in 96-well plate format was done in TECAN Infinite® M1000 Pro.

### Pre-Treatment of 96-Well Plates

As mentioned, in order to prevent non-specific binding of sEH or inhibitor on the 96-well plate, the 96 well plates were pre-incubated with PB with 0.1% gelatin overnight at room temperature. The gelatin coats the plate and prevents non-specific binding of sEH and sEH inhibitors to the plate. The buffer was discarded and the plate was dried before use.

### Kᵢ Assay Procedure:

The sEH stock was diluted to the desired concentration (20 nM) by PB (100 mM sodium phosphate, 0.1% gelatin, pH 7.4). ACPU (one equivalent to sEH, 10 mM, Ethanol) was added to the sEH solution and was incubated for 2 h at room temperature. The sEH-ACPU mixture (20 nM, 100 mM sodium phosphate, 0.1% gelatin, pH 7.4, 150 uL) was added to each well.

The baseline fluorescence (F₀) (λ_{excitation} at 280 nm, λₑₘᵢₛₛᵢₒₙ at 450 nm) of the samples was measured after the z-position and gain were optimized automatically by the fluorometers. The z and gain value was noted and will be used for the later fluorescent measurement. Because DMSO has been known to quench fluorescence, 1% DMSO in PB was served as a control (F_{DMSO}). The desired concentration of inhibitors which is the concentration that 100% of sEH was bound to inhibitor, was added at the first well and was further diluted by 2-fold across the rest of the wells. Based on our study, 12 datum points, which correspond to 12 different concentrations of the inhibitor, generated sufficient data to calculate the accurate Kᵢ for the inhibitors. The samples were incubated at 30° C. for 1.5 h. Then, the fluorescence (λ_{excitation} at 280 nm, λₑₘᵢₛₛᵢₒₙ at 450 nm) of the samples was measured using the z-position and gain values that were previously obtained. The obtained fluorescence signals were transformed as below and were used to calculate the Kᵢ of the inhibitors according to “Curve fitting” section below.

$$Initiated fluorescence = {F}_{DMSO ( well X )} / {F}_{0 ( well X )}$$

$$Saturated fluorescence = {F}_{at the saturated concentration ( well X )} / {F}_{0 ( at well X )}$$

$$Observed fluorescence = {F}_{( well X )} / {F}_{0 ( well X )}$$

### Curve Fitting

The curve fitting for Kᵢ determination was performed as reported in Lee et al. Forster resonance energy transfer competitive displacement assay for human soluble epoxide hydrolase. Anal Biochem. 2013; 434(2):259-68. The data manipulation and Kᵢ calculation were based on the original paper by Wang et al. An exact mathematical expression for describing competitive-binding of 2 different ligands to a protein molecule. Febs Letters. 1995; 360(2):111-4, with modifications suggested by Roehrl et al. Selective inhibition of calcineurin-nfat signaling by blocking protein-protein interaction with small organic molecules. Proceedings of the National Academy of Sciences of the United States of America. 2004; 101(20):7554-9, each of which is incorporated by reference in its entirety.

The displacement assay is based on a three-state equilibrium binding model. This is modeled as described below (Eq. 1)

$$[ RI ] + L ⇔ R + I + L ⇔ [ R L ] + I \qquad ( Eq . 1 )$$

with [RI] stands for receptor or enzyme-inhibitor complex;
L stands for reporting ligand;
I stands for inhibitors; and
[RL] stands for receptor or enzyme-reporting ligand complex.

The three-state equilibrium (Eq. 1) consists of the sEH-inhibitor complex, sEH and sEH-reporting ligand complex. In this study, the relative fluorescence intensity (F₃) was plotted against the concentration of sEH inhibitor and the resulting curve was fitted into equation (Eq. 2) derived by Wang et al. (cited above) for three-state equilibrium.

$$\text{?} = [ 2 {( \text{?} )}^{1 / 2} cos ( 6 / 3 ) - a ] / \{ 3 {K}_{d 1} + [ 2 {( \text{?} - 3 b )}^{1 / 2} cos ( \text{?} / 3 ) - a ] \qquad ( Eq . 2 )$$

$$with a - {K}_{d 1} + {K}_{ds} + L + I - R ;$$

$$b = \text{?} ( L - R ) + {K}_{d 1} ( 1 - R ) + {K}_{d 1} \text{?} ;$$

$$\text{?} = {K}_{d 1} {K}_{d 2} R ; and$$

$$θ = arccos \{ ( - 2 \text{?} + \text{?} - 27 \text{?} ) / [ 2 ( \text{?} ] \}$$

$$\text{?} \text{indicates text missing or illegible when filed}$$

where F₃=Relative Fluorescence=(observed fluorescence−fluorescence at saturation)/(initiated fluorescence−fluorescence at saturation)
I=the concentration of added unlabeled competing ligand;
R=the total concentration of sEH;
L=The total concentration of reporting ligand;
K_{d1}=the dissociation constant of reporting ligand (found by fluorescent binding assay);
K_{d2}=the inhibition constant of inhibitors; and
k_{off} measurement procedure.

The k_{off} measurement was run as described before in Lee et al. Optimized inhibitors of soluble epoxide hydrolase improve in vitro target residence time and in vivo efficacy. J Med Chem. 2014; 57(16):7016-30 and Lee et al. Forster resonance energy transfer competitive displacement assay for human soluble epoxide hydrolase. Anal Biochem. 2013; 434(2):259-68. The sEH (8 μM) was pre-incubated with the selected inhibitor (8.8 μM, 100 mM PB buffer, pH 7.4) for 1.5 h at room temperature. The sEH-inhibitor complex was then diluted 40 times with ACPU (20 μM, 100 mM Sodium phosphate buffer, pH 7.4). The fluorescence (λ_{excitation} at 280 nm, λₘᵢₛₛᵢₒₙ at 450 nm) was monitored immediately for every 30 s up to 5100 s. The fluorescence (λₑₘᵢₛₛᵢₒₙ at 450 nm) data was plotted against time (s). The resulting curve was fitted to single exponential growth and the relative k_{off} was obtained.

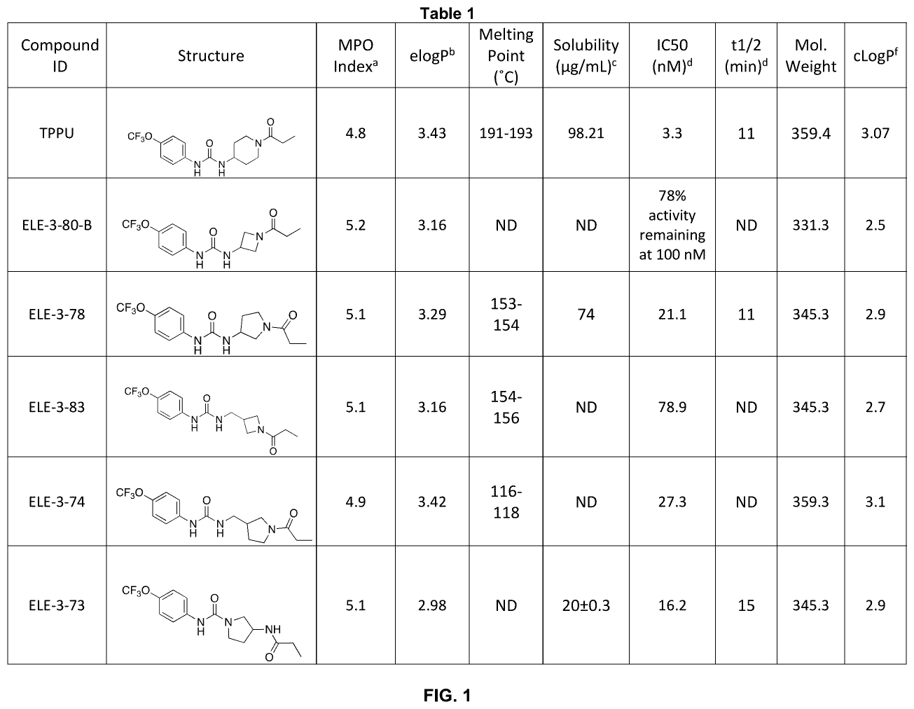

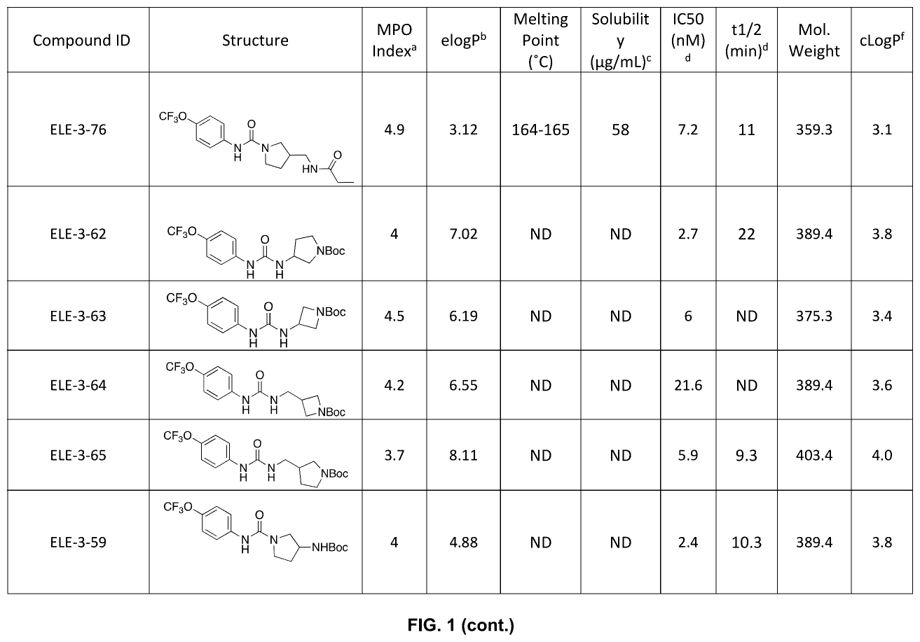

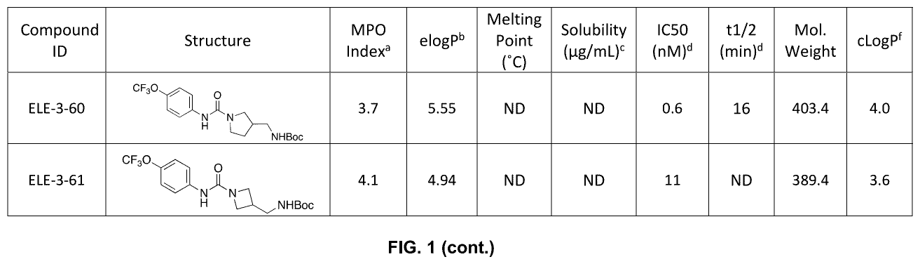

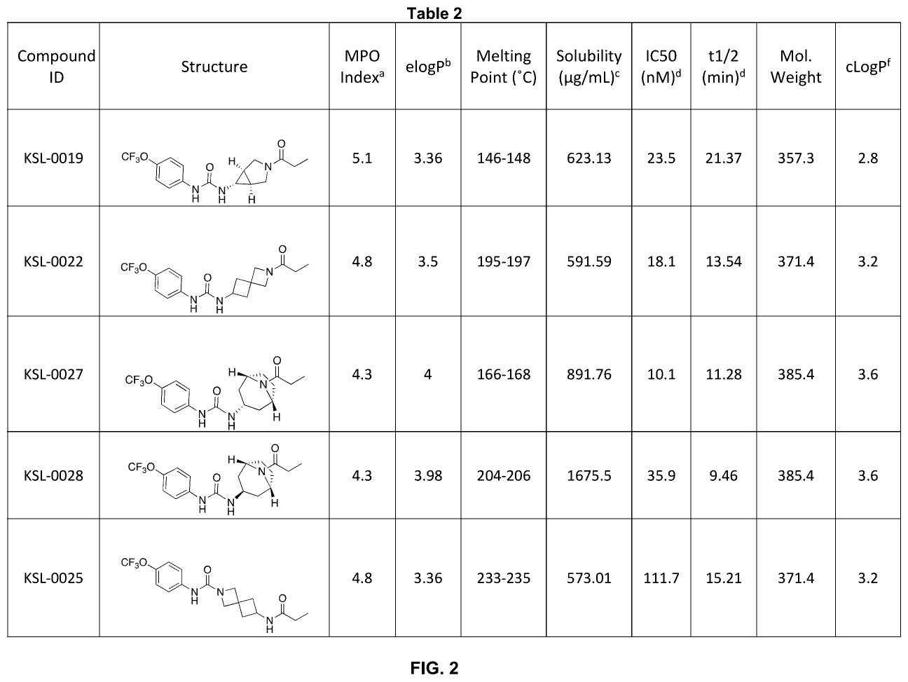

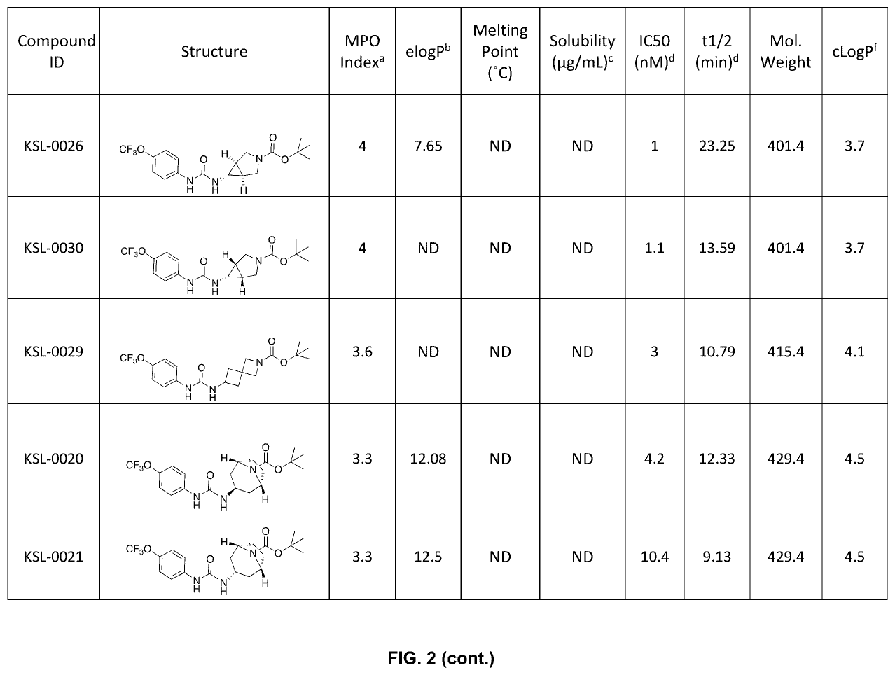

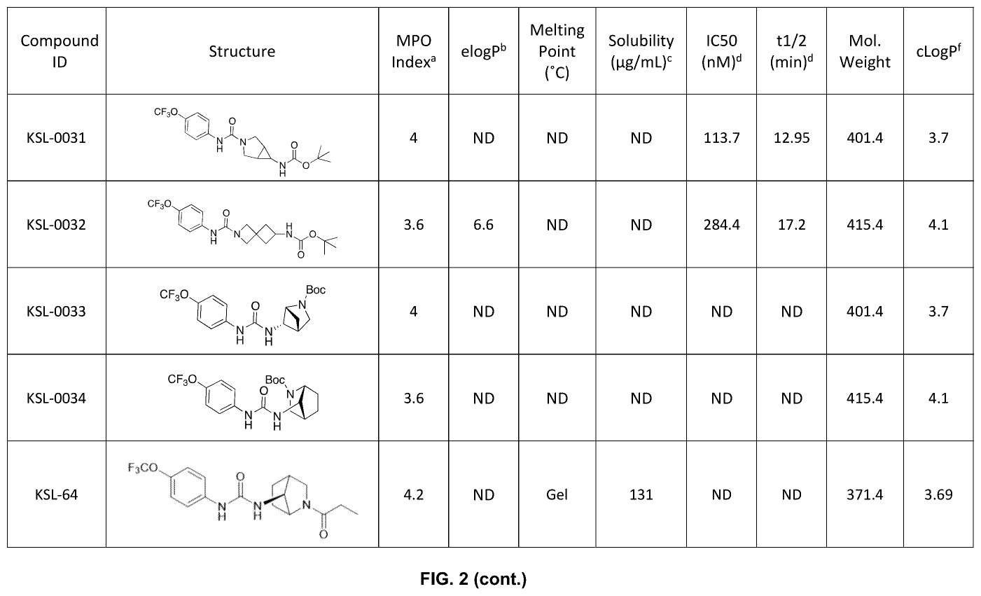

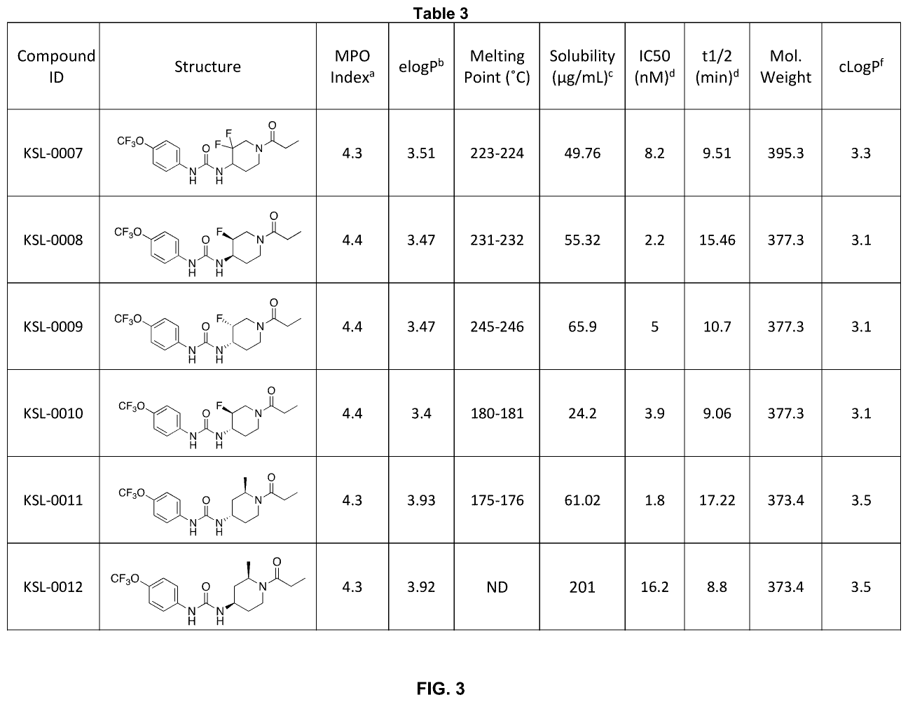

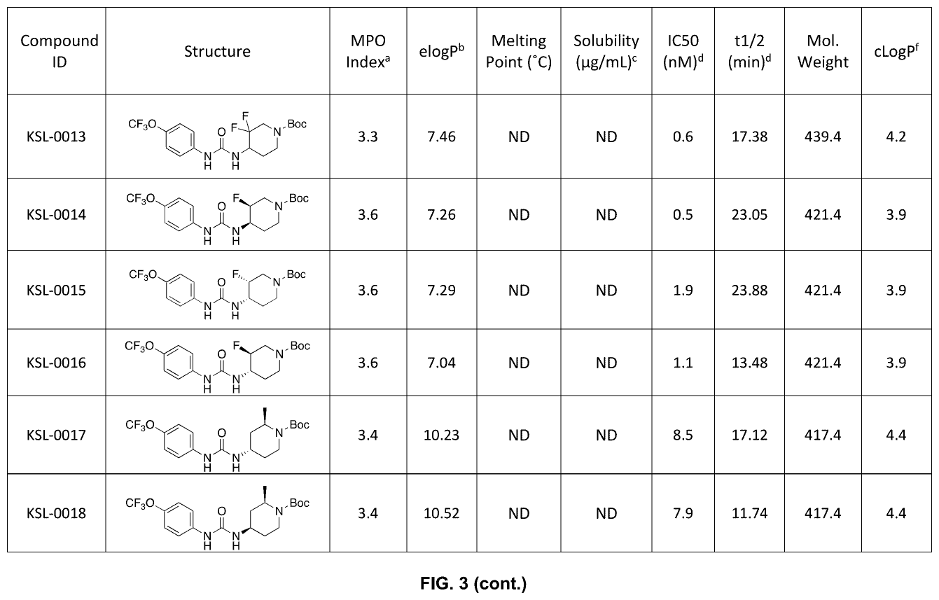

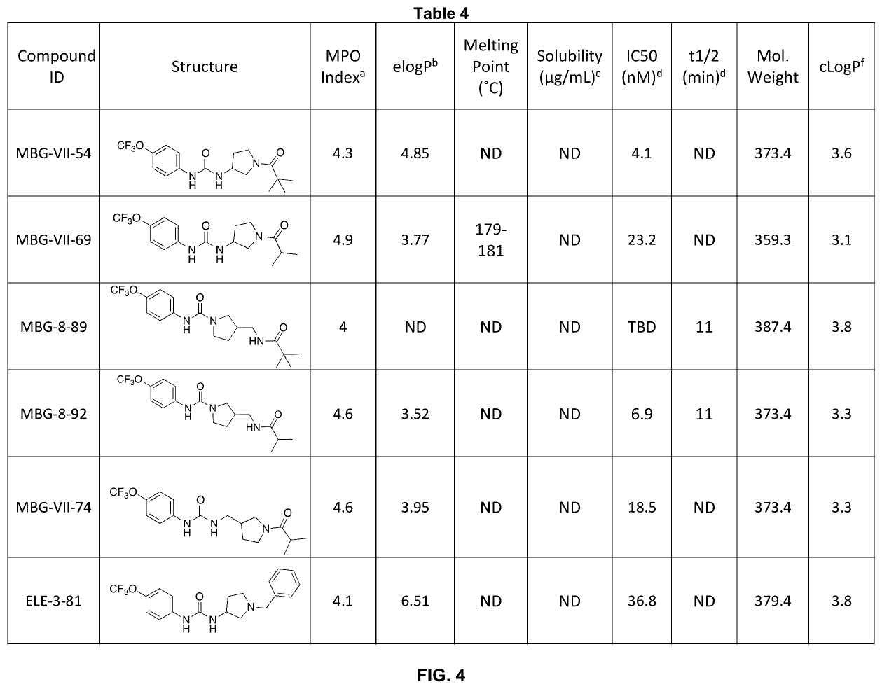

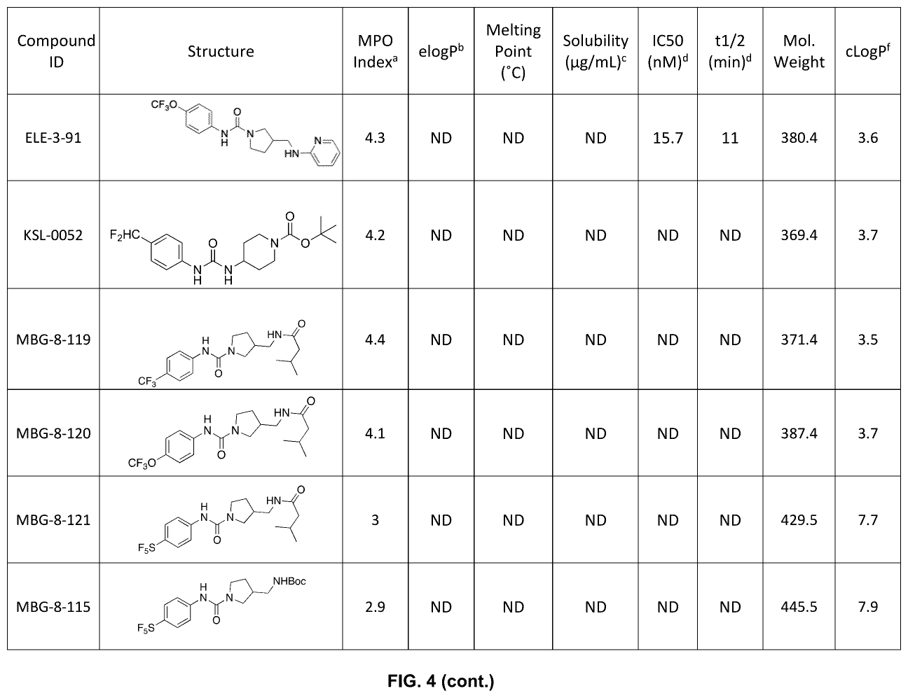

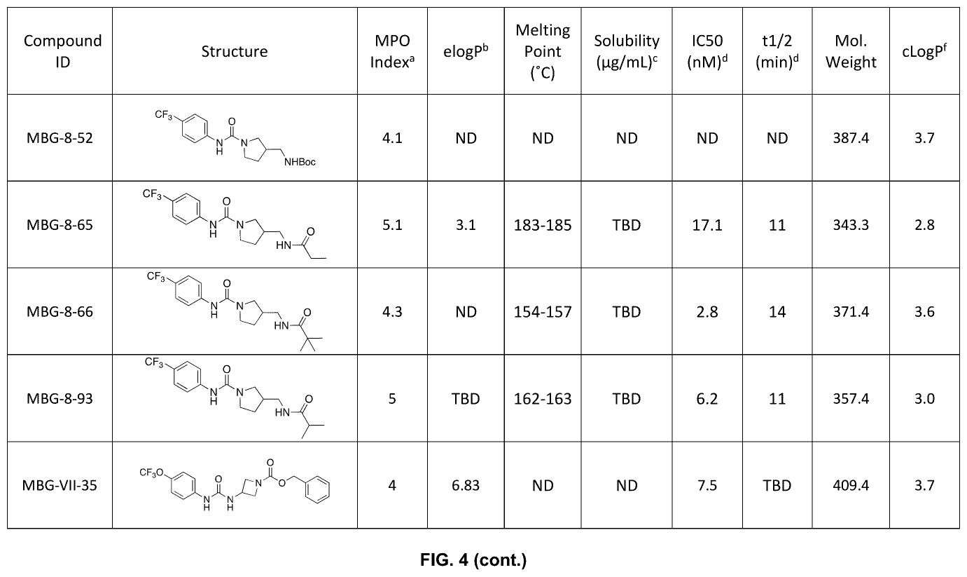

Results from Example 106 are presented in Tables 1-4 as FIGS. 1-4.

### Example 107: Murine Pharmacokinetic Study of Inhibitors Using Oral Dosing

All animal experiments were performed based on the published protocols approved by the Animal Use and Care Committee of Michigan State University. See Lee et al. Preparation and evaluation of soluble epoxide hydrolase inhibitors with improved physical properties and potencies for treating diabetic neuropathic pain. Bioorganic & Medicinal Chemistry. 2020; 28(22):115735; Lee et al. Drug-target residence time affects in vivo target occupancy through multiple pathways. ACS Cent Sci. 2019; 5(9):1614-241; and Lee et al. Optimized inhibitors of soluble epoxide hydrolase improve in vitro target residence time and in vivo efficacy. J Med Chem. 2014; 57(16):7016-30, each of which is incorporated by reference in its entirety.

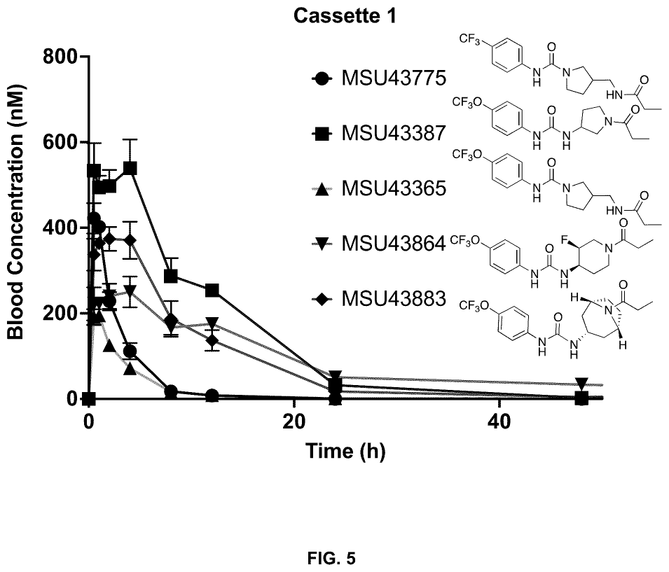

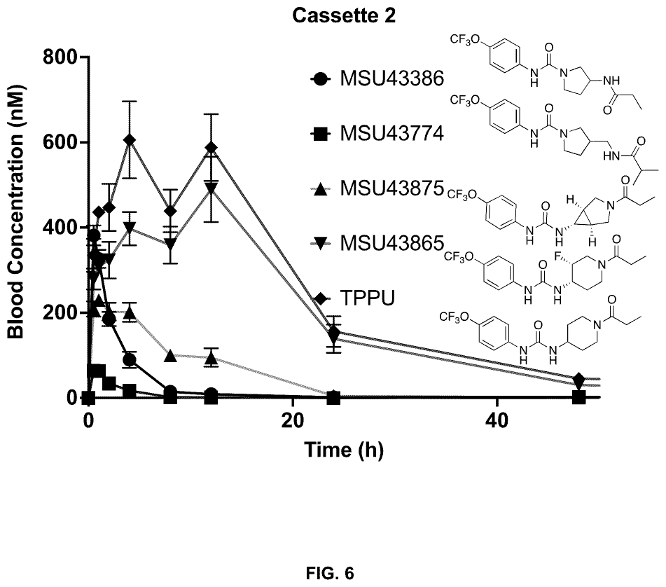

Ten to 12 week old, 20 to 36 g mice from Charles River Laboratories (C₅₇BL/6 mice (n=4 to 6)) were used in the pharmacokinetic studies. Selected inhibitors were dissolved in pure PEG400 at 40° C. overnight to give a clear solution. The inhibitor(s) (0.3 mg/kg, 100 μL) was administered orally in a cassette of 5 at 0.3 mg/kg dissolved in PEG400 at a concentration of 0.072 mg/mL per each inhibitor. The blood samples (5 μL) were then collected from tail vein using pipet tip pre-washed with 7.5% EDTA(K₃) at time 0, 0.5, 1, 2, 4, 8, 12, 24, 48 and 72 h after administration of the inhibitors. The collected blood samples were immediately transferred to an Eppendorf tube (1.5 mL) containing 50 μL of 0.1% EDTA (by weight) solution and mixed strongly. The samples were stored at −80° C. until analysis. The blood samples were then prepared based on the published procedure for LC-MS/MS analysis. See Lee et al. Optimized inhibitors of soluble epoxide hydrolase improved in vitro target residence time and in vivo efficacy. J Med Chem. 2014; 57(16):7016-30. Each group consists of 3-4 animals. The inhibitors' blood concentration was determined by HPLC/MS/MS as described in J Med Chem. 2014; 57(16):7016-30. The results of the pharmacokinetic studies are shown in FIGS. 5 and 6. The pharmacokinetic profile of newly synthesized sEH inhibitors indicated that the majority of the modifications around the linker of sEH inhibitors are well-tolerated. The pharmacokinetic studies on key compounds were repeated with administration as sole compounds rather than cassette. This procedure provides data indistinguishable from the cassette dose suggesting little drug-drug interaction on the pharmacokinetic profile.

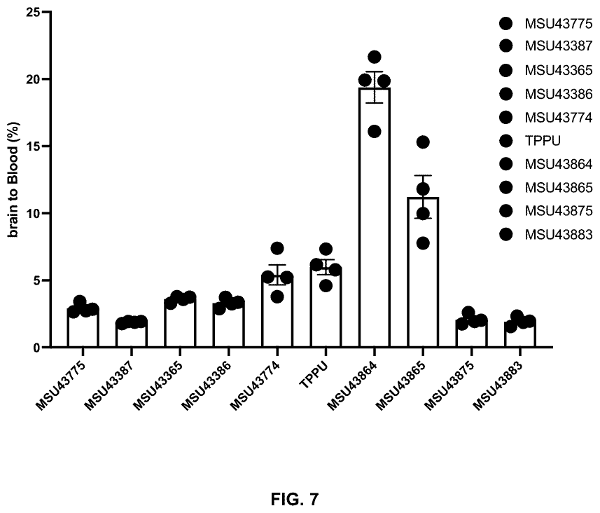

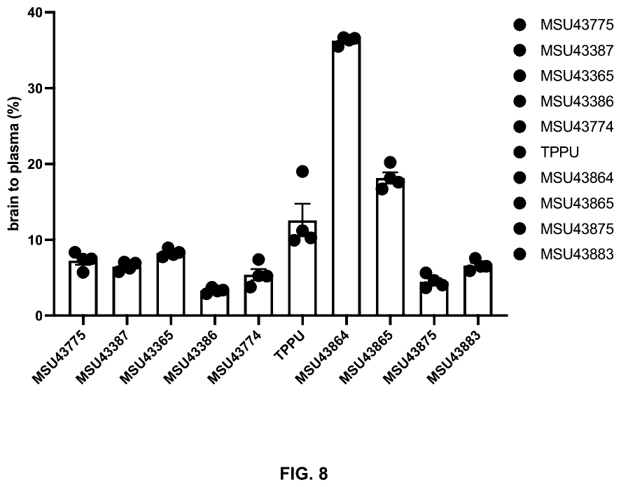

To determine the brain-to-blood ratio and brain-to-plasma ratio of the inhibitors, the mice were treated as described above with 0.3 mg/kg of inhibitor. The mice were euthanized at 4 h and the brains were extracted from the mice followed by snap freeze. The samples were stored at −78° C. until sample preparation. In parallel, the blood (500 μL) was collected by cardiac puncture and transferred to the sample collection tube containing 7.5% EDTA(K₃) (final concentration) and 10 uL antioxidant cocktail (containing triphenylphosphate and BHT). The plasma was separated by centrifugation. The collected plasma was snap-frozen and stored at −78° C. until sample preparation. The inhibitors' blood and brain concentration and plasma and brain concentration were determined by HPLC/MS/MS as described in J Med Chem. 2014; 57(16):7016-30. The inhibitors' brain-to-blood ratio is shown in FIG. 7, and the inhibitors' brain-to-plasma ratio is shown in FIG. 8. The results show that the CNS exposure of newly synthesized sEH inhibitors showed that fluorine substitution around the linker of sEH inhibitors substantially improved the brain-to-blood ratio and the brain-to-plasma ratio of sEH inhibitors.

### Analysis of sEH Inhibitors in Blood Samples (Mass Spec Tables)

The concentrations of inhibitors in the samples were determined based on the published LC/MSMS method by Liu et al. Substituted phenyl groups improve the pharmacokinetic profile and anti-inflammatory effect of urea-based soluble epoxide hydrolase inhibitors in murine models. Eur J Pharm Sci. 2013; 48(4-5):619-27, which is incorporated by reference in its entirety.

### Example 108: Tauopathy Murine Model

Mice (Tau P301S, Line PS19) are purchased from Jackson Lab and are treated with sEHI at 10 mg/kg/day mixed with standard chow for 6 months.

### Tau Protein Solubility and Phosphorylation States

Proteins are extracted from brains using the RAB-HS procedure: ice-cold high-salt reassembly buffer (RAB-HS, 0.1 M MES, 1 mM EGTA, 0.5 mM MgSO₄, 0.75 M NaCl, 0.02 M NaF, 1 mM PMSF, and 0.1% protease inhibitor cocktail) followed by centrifugation at 50,000×g for 40 min at 4° C.

The RAB-HS-insoluble pellets are re-homogenized with 1 M sucrose in RAB and centrifuged at 40,000 3 g for 20 min at 4° C. to remove myelin and associated lipids. The resulting pellets are dissolved with 1 ml/g tissue in RIPA (50 mM Tris, 150 mM NaCl, 0.1% SDS, 0.5% sodium deoxy-cholate, 1% NP40, and 5 mM EDTA [pH 8.0]) and centrifuged at 40,000 3 g for 20 min at 4° C. The supernatants are used as RIPA-soluble samples, while the RIPA-insoluble pellets are extracted with 70% FA to recover highly insoluble protein. Quantitative western blot analyses are performed.

### Immunohistochemical and Histochemical Studies

Mice are deeply anesthetized and transcardially perfused with 15 ml of phosphate-buffered saline (PBS), and brains and spinal cords are removed, followed by immersion fixation for 24 hr with 10% formalin in PBS and paraffin embedding. Six-micrometer paraffin sections of brains and spinal cords are immunostained using streptavidin-biotin-peroxidase as well as double- and triple-labeling immunofluorescence methods. For immunostaining microglia, 20 mm thick sections fixed with 4% paraformaldehyde are used, while Gallyas silver, thioflavin S, and Congo red methods are used to detect fibrillary tau lesions. To derive unbiased estimates of neuronal loss in the hippocampal CA3 region of PS19 Tg mice at 6 and 12 months of age, coronal sections sliced at the center of the mamillary body are used for quantitative analysis (n=4 per group). The number of pyramidal neurons in the CA3 region is measured manually using digital photomicrographs of H&E-stained preparations.

### Example 109: Transgenic Rat Model of Alzheimer's Disease

The procedure used is adopted from Kelly et al. Locus coeruleus degeneration induces forebrain vascular pathology in a transgenic rat model of alzheimer's disease. J Alzheimers Dis. 2019; 70(2):371-88, which is incoporated by reference in its entirety.

Male and female Tg344-19 AD rats on the Fischer 344 background, overexpressing both human amyloid-β precursor protein (AβPP) bearing the AβPP KM670/671NL Swedish mutation (APPswe) and human presenilin-1 (PS1) bearing the exon 9 deletion mutation (PS1ΔE9) under the mouse prion protein gene promoter are used [J Neurosci. 2013; 33(15):5245-56]. Tg344-19 AD rats manifest an age-dependent cerebral deposition of amyloid-β (Aβ) plaque-like pathology that precedes tauopathy, gliosis, apoptotic loss of neurons in the cerebral cortex and hippocampus, and cognitive disturbances [J Neurosci. 2013; 33(15):5245-56; Neurobiol Aging 2018; 61:169-176; Brain 2017; 140(11):3023-3038]. Original breeding colonies are provided by the Rat Resource Center (St. Louis, MO; funded by NIH grant P40 OD011062). Rats are bred by backcrossing hemizygous transgene positive animals to wild type Fischer 344 littermates. All animals are pair-housed in 12 h: 12 h reverse light-dark cycle conditions and ad libitum access to chow and water. The experimental timeline is as follows: 1) at six months of age, animals are administered IgG-sap or DBH-sap, 2) six weeks later, animals are behaviorally tested, and 3) following behavioral testing, the animals are sacrificed for postmortem studies. All procedures are conducted in accordance with guidelines set by the Institutional Animal Care and Use Committee of Michigan State University.

### Barnes Maze

Rats are evaluated behaviorally at six weeks after stereotactic surgeries. The investigator is blinded to the rat treatment group by a randomization table prior to testing and by coding the videos for all testing outcomes. All experimental sessions are recorded by a video camera placed above the apparatus and analyzed with video-tracking AnyMaze software (Stoelting, Wood Dale, IL). The software detects the center of the animal body and recorded distance moved and time active based on color differences between animal coat color and testing apparatus recorded by the camera. The short-protocol Barnes Maze for spatial and working memory function [J Comp Physiol Psychol 1979; 93:74-104] is adapted from Attar and colleagues [PLoS One 2013; 8:e80355]. Briefly, during the habituation session, the animals are slowly pulled to the escape hole in a clear cylinder and then given 120 s to freely enter the escape hole. Afterwards, the animals are submitted to a set of two daily training sessions, with at least two trials. The first training session is performed 24 h after the habituation session. All trials lasted 120 s or until the animals reach the escape box. However, if the rats do not reach the target hole, the experimenter gently guides the animal toward it at the end of the trial using a clear cylinder. After reaching the escape box, animals remain inside for at least 60 s before being returned to their home cages. The escape box is always located in the same place during training. Animals are tested in groups of four, in order that all trials average 20 min between each trial per animal. Retrieval of spatial learning is evaluated in the probe session, which is conducted after the rest day, 48 h after the last training day. The procedure is similar to the training trials, but the escape box is removed and rats are evaluated for 120 s. At the beginning of each session, the animals are placed in an opaque container at the center of the maze. The container is then pulled up, and the animal is released to explore the maze. Parameters analyzed in these experiments include time spent in target quadrant and latency to target hole entry (measures of spatial learning and memory) and incorrect revisits to holes already investigated (a measure of working or procedural memory). Incorrect revisits are defined as searching the same hole twice within a trial when the revisit occurred after the inspection of other holes [J Vis Exp 2014; e51194].

### Open Field Test

Locomotor activity is evaluated using a standard open field test [Psychol Bull 1976; 83:482-504]. The open field apparatus (Stoelting) consists of an open topped, 2times2 acrylic black box measuring 40.6×40.6×38 cm. The box is placed at table height and all experimental sessions are recorded by a video camera placed above the apparatus and analyzed with video-tracking software. All movements are automatically recorded and time mobile and distance traveled are plotted and the data is used to measure locomotor activity. On the day of testing, rats in their home cages are brought into the experimental room. Rats remain in the experimental room for 30 min, after which each rat is placed into the center of the observation box and recording begins immediately. Movement is recorded in 5-min bins for 30 min.

### Elevated Plus Maze

The open field test can also be used to measure anxiety [Behav Brain Res 2002; 134:49-57]. However, to ensure the DBH-sap lesion did not produce any anxiolytic or anxiogenic effects that might impact behavioral outcomes [J Psychopharmacol 2013; 27:659-693], the rats are tested on the elevated plus maze [Behav Brain Res 2002; 134:49-57]. The maze consists of four arms arranged in a plus shape, elevated 50 cm off the floor, with two arms opened as extend platforms away from the maze (open arms) and the other two arms as platforms with walls (closed arms). Rats are put in the center of the platform facing the same open arm and are allowed to explore the maze for 5 min. Time spent in open compared to closed arms is analyzed as a measure of relative fear/anxiety.

### Tissue Preparation

Twenty-four hours after completing the final behavior trials, rats are deeply anesthetized (pentobarbital, 60 mg/kg, i.p.) and perfused intracardially with 0.9% saline containing 10,000 USP/L heparin. Rat brains are immediately removed and hemisected in the sagittal plane at midline using a brain block. One hemisphere is post-fixed in 4% paraformaldehyde (Electron Microscopy Sciences, Hatfield, PA) in 0.1 M phosphate buffer (pH 7.2) for 24-48 h and processed for immunohistochemistry, whereas the other hemisphere is flash frozen and processed for biochemical analyses.

### Immunohistochemistry

Post-fixed brain hemispheres are transferred to 15% sucrose in 0.1 M phosphate buffer until saturated, then 30% sucrose in 0.1 M phosphate buffer until saturated. Brains are frozen on dry ice and sectioned at a 40 μm thickness in 1:12 series in the coronal plane using a freezing-sliding microtome (American Optical, Buffalo, NY). Serial sections are processed for immunohistochemistry (IHC) using the free-floating method [Acta Neuropathol Commun 2017; 5:8; J Neuropathol Exp Neurol 2006; 65:592-601; Curr Alzheimer Res 2014; 11:655-663]. Control experiments including primary antibody deletions are performed for each antibody. All antibodies an dilutions used in these experiments are listed in the table below:

TABLE 1
Antibodies used in the present study

| | | Dilution |
|---|---|---|
| Antibody | Source | (application) |
| DBH rabbit | Immunostar #22806 | 1:4000 (IHC) |
| polyclonal antiserum | | |
| albumin rabbit | ProteinTech | 1:500 (IHC) |
| polyclonal antiserum | #16475-1-AP | 1:10,000 (WB) |
| smooth muscle actin (ACTA2) | ProteinTech | 1:1000 (IHC) |
| rabbit polyclonal antiserum | #23081-1-AP | |
| MOAB-2 (Aβ 1-4) mouse | Gift from Dr. Nicholas | 1:4000 (IHC) |
| IgG2b monoclonal | Kanaan, Michigan | |
| antibody | State University | |
| GFAP rabbit polyclonal | Abcam #ab7260 | 1:10,000 (HC) |
| antiserum | | 1:20,000 (WB) |
| SMI71 mouse IgM | Biolegend #836804 | 1:1000 (IHC) |
| monoclonal antibody | | |
| AT8 (tau phospho-serine | Thermo Fisher | 1:100-1:5000 |
| 202, phospho-threonine 205) | #MN1020 | (IHC) |
| mouse IgG1 mouse | | |
| monoclonal antibody | | |
| CP-13 (tau phospho-serine | Gift from Dr. Peter | 1:100-1:5000 |
| 202) mouse IgG2b | Davies, Northwell | (IHC) |
| monoclonal antibody | Feinstein Institute | |

All publications mentioned in this specification are herein incorporated by reference. Various modifications and variations of the described compounds and methods described herein will be apparent to those skilled in the art without departing from the scope and spirit of the disclosure. Although the compounds and methods have been described in connection with specific preferred embodiments, it should be understood that the invention as claimed should not be unduly limited to such specific embodiments. Indeed, various modifications of the described modes for carrying out the claimed invention which are obvious to those skilled in biochemistry and biotechnology or related fields are intended to be within the scope of the claims.

It should be apparent to those skilled in the art that many more modifications besides those already described are possible without departing from the concepts herein. The inventive subject matter, therefore, is not to be restricted except in the spirit of the appended claims. “Comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

The invention claimed is:

1. A compound corresponding in structure to Formula I 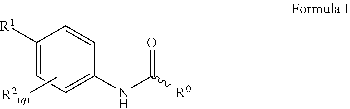
or a pharmaceutically acceptable salt thereof, wherein:
R⁰ is A, B, C or D, 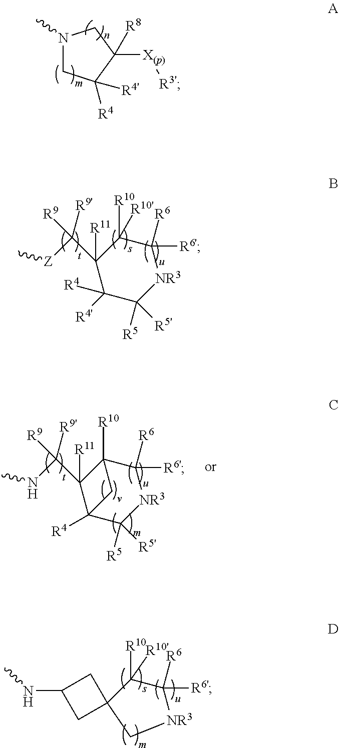
R¹ is selected from the group consisting of H, halo, —OCF₃, —OCF₂H, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, —CO-fluoroalkyl, aryl, and C₁-C₆-alkyl;
R^{3′} is selected from the group consisting of —NHCO-alkyl, —NHCO-aryl, —NHCO—O-alkyl, —NHCO-fluoroalkyl, —NCH₃CO—O-alkyl, —NCH₃CO-alkyl, —NH-aryl, —NH-alkyl, —NH₂, and C₁-C₆-alkoxy;
R⁴ and R^{4′} are independently selected from the group consisting of H, halo, and C₁-C₆-alkyl; or R⁴ and R^{4′} can together form a 3-6 membered ring; or R⁴ or R^{4′} can form a 3-6 membered ring with X; or R⁴ or R^{4′} can form a 3-6 membered ring with R^{3′}; or R⁴ or R^{4′} can form a 3-6 membered ring with R⁵ or R^{5′;}
X is selected from the group consisting of —C(R⁷)(R⁷); or X can form a 3-6 membered ring with R⁴ or R^{4′};
Z is NH or O;
R⁵ and R^{5′} are independently selected from the group consisting of H, halo, and C₁-C₆-alkyl; or R⁵ and R^{5′} can together form a 3-6 membered ring; or R⁵ or R^{5′} can form a 3-6 membered ring with R⁴ or R^{4′};
R⁶ and R^{6′} are independently selected from the group consisting of H, halo, and C₁-C₆-alkyl; or R⁶ or R^{6′} and can form a 3-6 membered ring with R⁴, R^{4′}, R⁵, or R^{5′}; or R⁶ and R^{6′} can together form a 3-6 membered ring;
R⁷ and R^{7′} are independently selected from the group consisting of H, C₁-C₆-alkyl, and substituted C₁-C₆-alkyl; or R⁷ and RT can together form a 3-6 membered ring;
R⁸ is H, halo, C₁-C₆-alkyl, C₁-C₆-alkoxy; or R⁸ can form a 3-6 membered ring with X; or R⁸ can form a 4-6 membered ring with X and R³;
R⁹ and R^{10′} are independently H, C₁-C₆-alkyl, and aryl;
R¹⁰ and R^{10′} are independently selected from the group consisting of H, halo, and C₁-C₆-alkyl or R¹⁰ or R^{10′} and can form a 3-6 membered ring with R⁴, R^{4′}, R⁵, or R^{5′;} or R¹⁰ or R^{10′} can together form a 3-6 membered ring;
R¹¹ is selected from the group consisting of H, halo, C₁-C₆-alkyl, and C₁-C₆-alkoxy;
m is zero, 1 or 2;
n is zero, 1 or 2;
p is zero, 1, 2, or 3;
q is zero, 1 or 2;
s is zero or 1;
t is zero or 1;
u is zero or 1;
v is zero, 1, or 2; and
wherein the compound is not 1-trifluoromethoxyphenyl-3-(1-propionylpiperidin-4-yl) urea (TPPU).

2. The compound or pharmaceutically acceptable salt of claim 1, wherein:
R¹ is selected from the group consisting of —OCF₃, —OCF₂H, —SF₅, —CF₃, —CHF₂, and —OCF₂;
R² is halo;
R³ is selected from the group consisting of CO-alkyl, —CO-fluoroalkyl, and CO—O-alkyl;
R^{3′} is selected from the group consisting of —NHCO-alkyl, —NHCO—O-alkyl, —NHCO-fluoroalkyl, —NCH₃CO—O-alkyl, —NCH₃CO-alkyl, —NH₂, and C₁-C₆-alkoxy;
R⁴ and R^{4′} are independently selected from the group consisting of H, and C₁-C₆-alkyl; or R⁴ and R^{4′} can together form a 3-6 membered ring; or R⁴ or R^{4′} can form a 3-6 membered ring with R^{3′;}
X is selected from the group consisting of C(R⁷)(R^{7′});
Z is NH or O;
R⁵ and R^{5′} are independently selected from the group consisting of H, halo, and C₁-C₆-alkyl;
R⁶ and R^{6′} are independently selected from the group consisting of H, halo, and C₁-C₆-alkyl;
R⁷ and RT are independently selected from the group consisting of H and C₁-C₆-R⁸ is H, C₁-C₆-alkyl, or C₁-C₆-alkoxy;
R⁹ and R^{9′} are independently H, and C₁-C₆-alkyl;
R¹⁰ and R^{10′} are independently selected from the group consisting of H, halo, and C₁-C₆-alkyl;
R¹¹ is selected from the group consisting of H, C₁-C₆-alkyl, and C₁-C₆-alkoxy;
m is zero, 1;
n is zero, 1;
p is zero or 1;
q is zero, 1 or 2;
s is zero or 1;
t is zero or 1;
u is zero or 1; and
v is zero, 1, or 2.

3. The compound or pharmaceutically acceptable salt of claim 1, wherein:
R⁰ is A, B or C;
R¹ is selected from the group consisting of —OCF₃, —SF₅, and —CF₃;
R² is halo;
R³ is selected from the group consisting of CO-alkyl and CO—O-alkyl;
R^{3′} is selected from the group consisting of —NHCO-alkyl and —NHCO—O-alkyl;
R⁴ and R^{4′} are independently selected from the group consisting of H, and C₁-C₆-alkyl; or R⁴ and R^{4′} can together form a 3-6 membered ring;
X is selected from the group consisting of C(R⁷)(R^{7′});
Z is NH;
R⁵ and R^{5′} are independently selected from the group consisting of H, halo, and C₁-C₆-alkyl;
R⁶ and R^{6′} are independently selected from the group consisting of H, halo, and C₁-C₆-alkyl;
R⁷ and RT are independently selected from the group consisting of H and C₁-C₆-R⁸ is H, C₁-C₆-alkyl, or C₁-C₆-alkoxy;
R⁹ and R^{9′} are independently H, and C₁-C₆-alkyl;
R¹⁰ and R^{10′} are independently selected from the group consisting of H, halo, and C₁-C₆-alkyl;
R¹¹ is selected from the group consisting of H, C₁-C₆-alkyl, and C₁-C₆-alkoxy;
m is zero, 1;
n is zero, 1;
p is zero or 1;
q is zero, 1 or 2;
s is zero or 1;
t is zero or 1;
u is zero or 1; and
v is zero, 1, or 2.

4. The compound or pharmaceutically acceptable salt of claim 1, wherein:
R⁰ is A, B or C;
R¹ is selected from the group consisting of —OCF₃ and —SF₅;
R³ is selected from the group consisting of CO-alkyl and CO—O-alkyl;
R^{3′} is selected from the group consisting of —NHCO-alkyl and —NHCO—O-alkyl;
R⁴ and R^{4′} are independently selected from the group consisting of H and C₁-C₆-alkyl;
X is selected from the group consisting of C(R⁷)(R^{7′});
Z is NH;
R⁵ and R^{5′} are independently selected from the group consisting of H, halo, and C₁-C₆-alkyl;
R⁶ and R^{6′} are independently selected from the group consisting of H, halo, and C₁-C₆-alkyl;
R⁷ and R^{7′} are independently selected from the group consisting of H and C₁-C₆-R⁸ is H;
R⁹ and R^{9′} are independently H, and C₁-C₆-alkyl;
R¹⁰ and R^{10′} are independently selected from the group consisting of H, halo, and C₁-C₆-alkyl;
R¹¹ is selected from the group consisting of H, C₁-C₆-alkyl, and C₁-C₆-alkoxy;
m is zero, 1;
n is zero, 1;
p is zero or 1;
q is zero;
s is zero or 1;
t is zero or 1;
u is zero or 1; and
v is zero, 1, or 2.

5. The compound or pharmaceutically acceptable salt of claim 1, wherein:
R⁰ is A 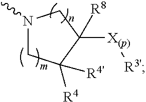
and
R¹ is selected from the group consisting of H, halo, —OCF₃, —CF₃, C₁-C₆-alkyl, —CF(CF₃)₂, —SF₅, —CHF₂, and —OCF₂;
R² is halo;
R^{3′} is selected from the group consisting of —NHCO-alkyl, —NHCO-aryl, —NHCO—O-alkyl, —NHCO-fluoroalkyl, —NCH₃CO—O-alkyl, —NCH₃CO-alkyl, —NH-aryl, —NH-alkyl, —NH₂, and C₁-C₆-alkoxy;
R⁴ and R^{4′} are independently selected from the group consisting of H, halo, and C₁-C₆-alkyl; or R⁴ and R^{4′} can together form a 3-6 membered ring; or R⁴ or R^{4′} can form a 3-6 membered ring with X; or R⁴ or R^{4′} can form a 3-6 membered ring with R³;
X is selected from the group consisting of —C(R⁷)(R⁷); or X can form a 3-6 membered ring with R⁴ or R^{4′};
R⁷ and RT are independently selected from the group consisting of H, C₁-C₆-alkyl, and substituted C₁-C₆-alkyl; or R⁷ and R^{7′} can together form a 3-6 membered ring;
R⁸ is H, halo, C₁-C₆-alkyl, C₁-C₆-alkoxy; or R⁸ can form a 3-6 membered ring with X; or R⁸ can form a 4-6 membered ring with X and R^{3′;}
m is zero, 1 or 2;
n is zero, 1 or 2;
p is zero, 1, 2, or 3; and
q is zero, 1 or 2.

6. The compound or pharmaceutically acceptable salt of claim 1, wherein:
R⁰ is B 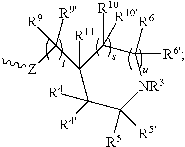
and
R¹ is selected from the group consisting of H, halo, —OCF₃, —OCF₂H, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, —CO-fluoroalkyl, aryl, and C₁-C₆-alkyl;
R⁴ and R^{4′} are independently selected from the group consisting of H, halo, and C₁-C₆-alkyl; or R⁴ and R^{4′} can together form a 3-6 membered ring;
Z is NH or O;
R⁵ and R^{5′} are independently selected from the group consisting of H, halo, and C₁-C₆-alkyl; or R⁵ or R^{5′} can form a 3-6 membered ring with R⁴ or R^{4′};
R⁶ and R^{6′} are independently selected from the group consisting of H, halo, and C₁-C₆-alkyl; or R⁶ or R^{6′} and can form a 3-6 membered ring with R⁴, R^{4′}, R⁵, or R^{5′}; or
R⁶ and R^{6′} can together form a 3-6 membered ring;
R⁹ and R^{9′} are independently H, C₁-C₆-alkyl, and aryl;
R¹⁰ and R^{10′} are independently selected from the group consisting of H, halo, and C₁-C₆-alkyl or R¹⁰ or R^{10′} and can form a 3-6 membered ring with R⁴, R^{4′}, R⁵, or R^{5′;} or R¹⁰ or R^{10′} can together form a 3-6 membered ring;
R¹¹ is selected from the group consisting of H, halo, C₁-C₆-alkyl, and C₁-C₆-alkoxy;
q is zero, 1 or 2;
s is zero or 1;
t is zero or 1; and
u is zero or 1.

7. The compound or pharmaceutically acceptable salt of claim 1, wherein:
R⁰ is C 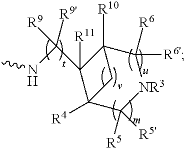
and
R¹ is selected from the group consisting of H, halo, —OCF₃, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, aryl, and C₁-C₆-alkyl;
R⁴ is selected from the group consisting of H, halo, and C₁-C₆-alkyl;
R⁵ and R^{5′} are independently selected from the group consisting of H, halo, and C₁-C₆-alkyl; or R⁵ or R^{5′} can form a 3-6 membered ring with R⁴;
R⁶ and R^{6′} are independently selected from the group consisting of H, halo, and C₁-C₆-alkyl; or R⁶ or R^{6′} and can form a 3-6 membered ring with R⁴, R⁵, or R^{5′}; or R⁶ and R^{6′} can together form a 3-6 membered ring;
R⁹ and R^{9′} are independently H, C₁-C₆-alkyl, and aryl;
R¹⁰ is selected from the group consisting of H, halo, and C₁-C₆-alkyl or R¹⁰ and can form a 3-6 membered ring with R⁴, R⁵, or R^{5′;}
R¹¹ is selected from the group consisting of H, halo, C₁-C₆-alkyl, and C₁-C₆-alkoxy;
m is zero, 1 or 2;
q is zero, 1 or 2;
t is zero or 1;
u is zero or 1; and
v is zero, 1, or 2.

8. The compound or pharmaceutically acceptable salt of claim 1, wherein:
R^{o} is D 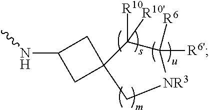
and
R¹ is selected from the group consisting of H, halo, —OCF₃, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, aryl, and C₁-C₆-alkyl;
R⁶ and R^{6′} are independently selected from the group consisting of H, halo, and C₁-C₆-alkyl; or R⁶ and R^{6′} can together form a 3-6 membered ring;
R¹⁰ and R^{10′} are independently selected from the group consisting of H, halo, and C₁-C₆-alkyl; or R¹⁰ or R^{10′} can together form a 3-6 membered ring;
m is zero, 1 or 2;
q is zero, 1 or 2;
s is zero or 1; and
u is zero or 1.

9. The compound or pharmaceutically acceptable salt of claim 1, wherein the compound is selected from the group consisting of:

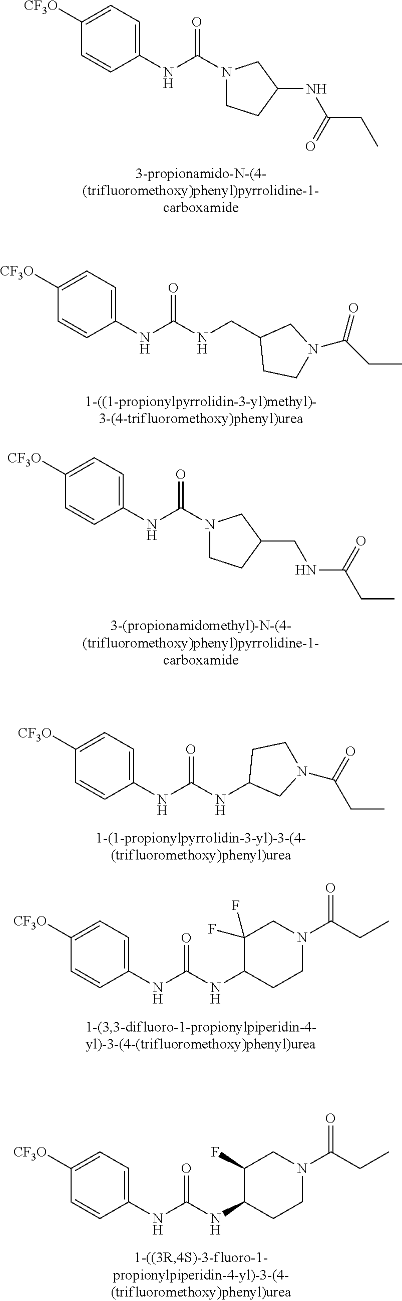

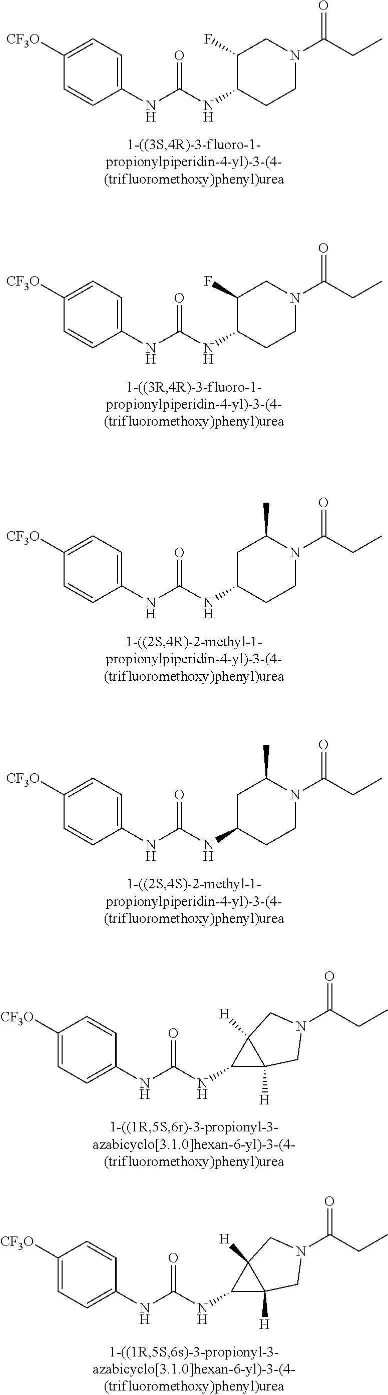

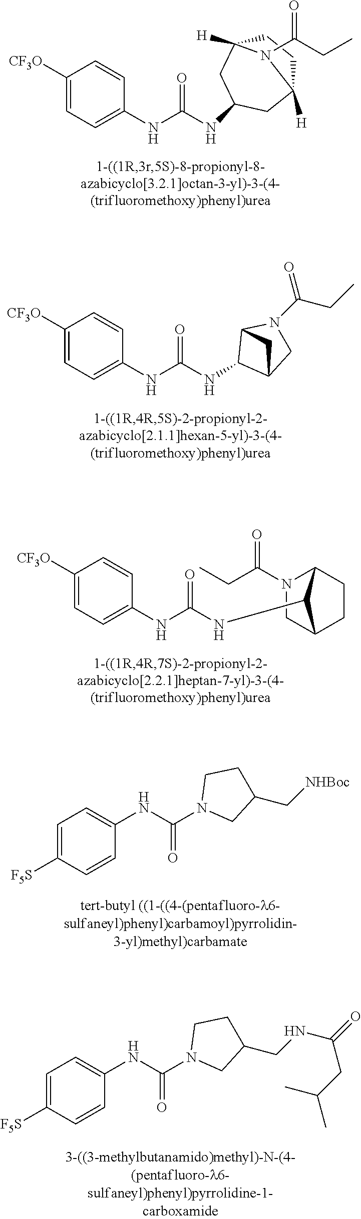

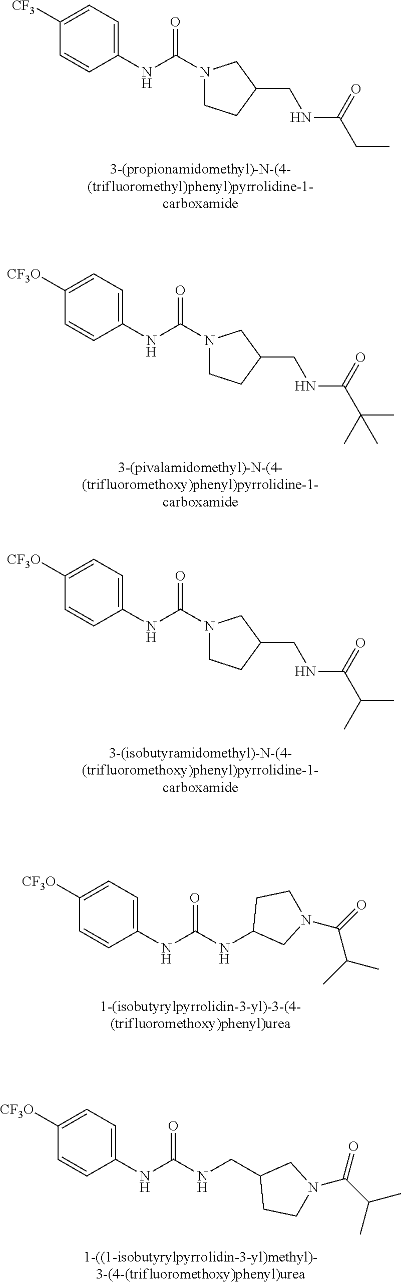

10. A pharmaceutical composition comprising a compound or pharmaceutically acceptable salt thereof of claim 1 and a pharmaceutically acceptable carrier.

11. The pharmaceutical composition of claim 10, wherein the pharmaceutical composition is adapted for oral, intravenous, intramuscular, subcutaneous, nasal, rectal, buccal or transdermal administration to a subject.

12. A method of preventing, delaying or reducing the occurrence of, or treating Alzheimer's disease in a subject in need thereof, the method comprising administering to the subject at least one compound or pharmaceutically acceptable salt corresponding in structure to Formula I 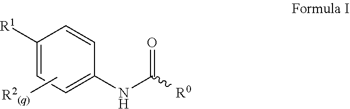
or a pharmaceutically acceptable salt thereof, wherein:
R⁰ is A, B, C or D, 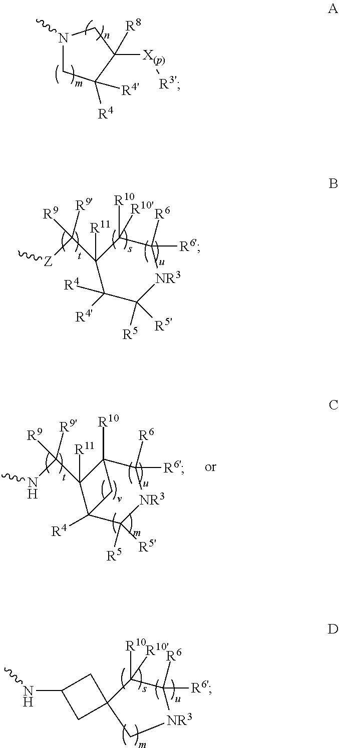
R¹ is selected from the group consisting of H, halo, —OCF₃, —OCF₂H, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, —CO-fluoroalkyl, aryl, and C₁-C₆-alkyl;
R^{3′} is selected from the group consisting of —NHCO-alkyl, —NHCO-aryl, —NHCO—O-alkyl, —NHCO-fluoroalkyl, —NCH₃CO—O-alkyl, —NCH₃CO-alkyl, —NH-aryl, —NH-alkyl, —NH₂, and C₁-C₆-alkoxy;
R⁴ and R^{4′} are independently selected from the group consisting of H, halo, and C₁-C₆-alkyl; or R⁴ and R^{4′} can together form a 3-6 membered ring; or R⁴ or R^{4′} can form a 3-6 membered ring with X; or R⁴ or R^{4′} can form a 3-6 membered ring with R^{3′}; or R⁴ or R^{4′} can form a 3-6 membered ring with R⁵ or R^{5′}
X is selected from the group consisting of —C(R⁷)(R⁷); or X can form a 3-6 membered ring with R⁴ or R^{4′;}
Z is NH or O;
R⁵ and R^{5′} are independently selected from the group consisting of H, halo, and C₁-C₆-alkyl; or R⁵ and R^{5′} can together form a 3-6 membered ring; or R⁵ or R^{5′} can form a 3-6 membered ring with R⁴ or R^{4′;}
R⁶ and R^{6′} are independently selected from the group consisting of H, halo, and C₁-C₆-alkyl; or R⁶ or R^{6′} and can form a 3-6 membered ring with R⁴, R^{4′}, R⁵, or R^{5′}; or R⁶ and R^{6′} can together form a 3-6 membered ring;
R⁷ and R^{7′} are independently selected from the group consisting of H, C₁-C₆-alkyl, and substituted C₁-C₆-alkyl; or R⁷ and RT can together form a 3-6 membered ring;
R⁸ is H, halo, C₁-C₆-alkyl, C₁-C₆-alkoxy; or R⁸ can form a 3-6 membered ring with X; or R⁸ can form a 4-6 membered ring with X and R^{3′;}
R⁹ and R^{9′} are independently H, C₁-C₆-alkyl, and aryl;
R¹⁰ and R^{10′} are independently selected from the group consisting of H, halo, and C₁-C₆-alkyl or R¹⁰ or R^{10′} and can form a 3-6 membered ring with R⁴, R^{4′}, R⁵ or R^{5′;} or R¹⁰ or R^{10′} can together form a 3-6 membered ring;
R¹¹ is selected from the group consisting of H, halo, C₁-C₆-alkyl, and C₁-C₆-alkoxy;
m is zero, 1 or 2;
n is zero, 1 or 2;
p is zero, 1, 2, or 3;
q is zero, 1 or 2;
s is zero or 1;
t is zero or 1;
u is zero or 1;
v is zero, 1, or 2; and
wherein the compound is not 1-trifluoromethoxyphenyl-3-(1-propionylpiperidin-4-yl) urea (TPPU).

13. A method of treating brain injury inflammation, the method comprising administering to the subject at least one compound or pharmaceutically acceptable salt corresponding in structure to Formula I 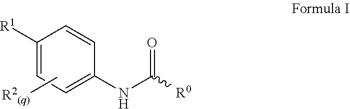
or a pharmaceutically acceptable salt thereof, wherein:
R⁰ is A, B, C or D, 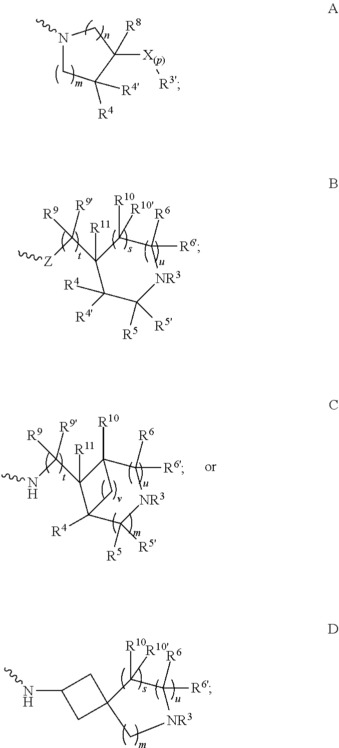
R¹ is selected from the group consisting of H, halo, —OCF₃, —OCF₂H, —CF₃, C₁-C₆-alkyl, —CF(CF₃)₂, —SF₅, —CHF₂, and —OCF₂;
R² is halo;
R³ is selected from the group consisting of —CO-alkyl, —CO-aryl, —CO—O-alkyl, —CO—O-alkylaryl, —CO-fluoroalkyl, aryl, and C₁-C₆-alkyl;
R^{3′} is selected from the group consisting of —NHCO-alkyl, —NHCO-aryl, —NHCO—O-alkyl, —NHCO-fluoroalkyl, —NCH₃CO—O-alkyl, —NCH₃CO-alkyl, —NH-aryl, —NH-alkyl, —NH₂, and C₁-C₆-alkoxy;
R⁴ and R^{4′} are independently selected from the group consisting of H, halo, and C₁-C₆-alkyl; or R⁴ and R^{4′} can together form a 3-6 membered ring; or R⁴ or R^{4′} can form a 3-6 membered ring with X; or R⁴ or R^{4′} can form a 3-6 membered ring with R^{3′}; or R⁴ or R^{4′} can form a 3-6 membered ring with R⁵ or R^{5′;}
X is selected from the group consisting of —C(R⁷)(R⁷); or X can form a 3-6 membered ring with R⁴ or R^{4′;}
Z is NH or O;
R⁵ and R^{5′} are independently selected from the group consisting of H, halo, and C₁-C₆-alkyl; or R⁵ and R^{5′} can together form a 3-6 membered ring; or R⁵ or R^{5′} can form a 3-6 membered ring with R⁴ or R^{4′;}
R⁶ and R^{6′} are independently selected from the group consisting of H, halo, and
C₁-C₆-alkyl; or R⁶ or R^{6′} and can form a 3-6 membered ring with R⁴, R^{4′}, R⁵, or
R^{5′}; or R⁶ and R^{6′} can together form a 3-6 membered ring;
R⁷ and RT are independently selected from the group consisting of H, C₁-C₆-alkyl, and substituted C₁-C₆-alkyl; or R⁷ and R^{7′} can together form a 3-6 membered ring;
R⁸ is H, halo, C₁-C₆-alkyl, C₁-C₆-alkoxy; or R⁸ can form a 3-6 membered ring with X; or R⁸ can form a 4-6 membered ring with X and R^{3′;}
R⁹ and R^{9′} are independently H, C₁-C₆-alkyl, and aryl;
R¹⁰ and R^{10′} are independently selected from the group consisting of H, halo, and C₁-C₆-alkyl or R¹⁰ or R^{10′} and can form a 3-6 membered ring with R⁴, R^{4′}, R⁵, or R^{5′;}
or R¹⁰ or R^{10′} can together form a 3-6 membered ring;
R¹¹ is selected from the group consisting of H, halo, C₁-C₆-alkyl, and C₁-C₆-alkoxy;
m is zero, 1 or 2;
n is zero, 1 or 2;
p is zero, 1, 2, or 3;
g is zero, 1 or 2;
s is zero or 1;
t is zero or 1;
u is zero or 1;
v is zero, 1, or 2; and
wherein the compound is not 1-trifluoromethoxyphenyl-3-(1-propionylpiperidin-4-yl) urea (TPPU).

14. The method of claim 12, wherein the administration is oral, intravenous, intramuscular, subcutaneous, nasal, rectal, buccal or transdermal.

15. The method of claim 13, wherein the administration is oral, intravenous, intramuscular, subcutaneous, nasal, rectal, buccal or transdermal.
